(19) United States
(12) Patent Application Publication
(10) Pub. No.: US 2025/0057883 A1
(43) Pub. Date: Feb. 20, 2025

(54) CONTROLLABLE STIMULATION OF GENETICALLY ENGINEERED LYMPHOCYTES FOR THE TREATMENT OF CANCER

(71) Applicant: THE TRUSTEES OF THE UNIVERSITY OF PENNSYLVANIA, Philadelphia, PA (US)

(72) Inventor: Saar GILL, Philadelphia, PA (US)

(21) Appl. No.: 18/723,240

(22) PCT Filed: Dec. 22, 2022
(86) PCT No.: PCT/US2022/082236

§ 371 Date: Jun. 21, 2024

Related U.S. Application Data
(60) Provisional application No. 63/293,417, filed on Dec. 23, 2021.

(51) Int. Cl. A61K 35/17; A61K 38/00; A61K 39/00; C07K 14/535; C07K 14/54; C07K 14/715; C07K 14/725; C12N 5/0783; C12N 15/86

(52) U.S. Cl. CPC A61K 35/17; A61K 39/4611; A61K 39/4631; A61K 39/4637; A61K 39/464424; C07K 14/535; C07K 14/5443; C07K 14/7051; C07K 14/7153; C07K 14/7155; C12N 5/0636; C12N 15/86; A61K 38/00; A61K 2239/13; A61K 2239/21; C07K 2317/622; C07K 2319/03; C12N 2510/00; C12N 2740/15043

(57) ABSTRACT

The present disclosure provides methods for controllable stimulation of genetically engineered lymphocytes. Compositions and methods of treatment are also provided.

### CROSS-REFERENCE TO RELATED APPLICATION

The present application is entitled to priority under 35 U.S.C. § 119(e) to U.S. Provisional Patent Application No. 63/293,417 filed Dec. 23, 2021, which is hereby incorporated by reference in its entirety herein.

### BACKGROUND OF THE INVENTION

There is a need in the art for new methods that control the activation (proliferation, expansion, and activity) of genetically engineered T cells (e.g. CAR T cells). The present invention addresses this need.

### SUMMARY OF THE INVENTION

In one aspect, the invention provides a method of controlling T cell expansion in a subject. The method comprises administering to the subject a modified T cell comprising an ectopic Granulocyte Colony Stimulating Factor receptor (G-CSFR), administering Granulocyte Colony Stimulating Factor (G-CSF) to the subject to stimulate expansion of the T cell, and/or administering an inhibitor of G-CSF, JAK 2, or JAK1/2 to the subject to inhibit expansion of the T cell.

In certain embodiments, the G-CSFR is an alternatively spliced class IV G-CSFR. In certain embodiments, the G-CSF is recombinant human G-CSF (rhG-CSF).

In certain embodiments, the T cell comprises a chimeric antigen receptor (CAR).

Another aspect of the invention includes a modified immune cell or precursor cell thereof, comprising a chimeric antigen receptor (CAR) and an ectopic/exogeneous G-CSF receptor (G-CSFR).

In certain embodiments, the G-CSFR is an alternatively spliced class IV G-CSFR. In certain embodiments, the G-CSFR comprises a deletion at aa 715 (GCSFRd715).

In certain embodiments, the immune cell or precursor cell thereof is a T cell. In certain embodiments, the T cell is a human T cell. In certain embodiments, the cell is an autologous cell.

Another aspect of the invention includes a method of treating a disease or disorder in a subject in need thereof, comprising administering to the subject a modified T cell comprising a CAR and an ectopic G-CSFR.

In certain embodiments, the disease or disorder is cancer.

In certain embodiments, the method further comprises administering G-CSF to the subject to stimulate expansion of the T cell.

In certain embodiments, the method further comprises administering an inhibitor of G-CSF, JAK 2, or JAK1/2 to the subject to inhibit expansion of the T cell.

In certain embodiments, the method is used to prevent chemotherapy-associated neutropenia.

In certain embodiments, the G-CSFR is an alternatively spliced class IV G-CSFR. In certain embodiments, the G-CSFR comprises a deletion at aa 715 (GCSFRd715).

In certain embodiments, the G-CSF is recombinant rhG-CSF.

Another aspect of the invention includes a method of generating a population of cells for use as an immunotherapy. The method comprises introducing into an immune cell or precursor cell thereof a measles virus (MV) pseudotyped lentiviral vector encoding a membrane bound version of IL15 (mbIL15) and a chimeric antigen receptor (CAR).

In certain embodiments, the immune cell or precursor cell thereof is a T cell. In certain embodiments, the T cell is human T cell. In certain embodiments, the T cell is an autologous T cell.

In certain embodiments, the mbIL15 comprises a fusion protein comprising IL15 linked to IL15Rα via a flexible linker.

In certain embodiments, method generates a population of cells that is 1-, 2-, 5-, or 10-fold higher in cell number compared to a method that does not comprise a measles virus (MV) pseudotyped lentiviral vector encoding mbIL15.

Another aspect of the invention includes a method of expanding CAR T cells. The method comprises introducing into the CAR T cell a measles virus (MV) pseudotyped lentiviral vector encoding a membrane bound version of IL15 (mbIL15).

In certain embodiments, the mbIL15 comprises a fusion protein comprising IL15 linked to IL15Rα via a flexible linker.

In certain embodiments, the method generates a population of cells that is 1-, 2-, 5-, or 10-fold higher in cell number compared to a method that does not comprise a measles virus (MV) pseudotyped lentiviral vector encoding mbIL15.

Another aspect of the invention includes a method of treating cancer in a subject in need thereof. The method comprises administering to the subject a population of cells generated by any of the methods contemplated herein or any of the expanded CAR T cells contemplated herein.

Another aspect of the invention includes a composition comprising a measles virus (MV) pseudotyped lentiviral vector comprising a nucleotide sequence encoding membrane-bound IL15 and a nucleotide sequence encoding a chimeric antigen receptor (CAR).

In certain embodiments, the vector comprises a nucleotide sequence at least 85%, 90%, 95%, 95%, 96%, 97%, 98%, 99%, or 100% identical to SEQ ID NO: 1.

Another aspect of the invention includes a composition comprising a measles virus (MV) pseudotyped lentiviral vector comprising a nucleotide sequence encoding CSF3R and a nucleotide sequence encoding a chimeric antigen receptor (CAR).

In certain embodiments, the vector comprises a nucleotide sequence at least 85%, 90%, 95%, 95%, 96%, 97%, 98%, 99%, or 100% identical to SEQ ID NOs: 2-4.

In certain embodiments, the CAR comprises an antigen binding domain, a transmembrane domain, and an intracellular domain. In certain embodiments, the antigen binding domain is selected from the group consisting of an antibody, an scFv, and a Fab. In certain embodiments, the antigen binding domain is capable of binding a tumor associated antigen (TAA).

### BRIEF DESCRIPTION OF THE DRAWINGS

The foregoing and other features and advantages of the present invention will be more fully understood from the following detailed description of illustrative embodiments taken in conjunction with the accompanying drawings.

FIGS. 1A-1E: Transgenic IL15 and IL15Ra-IL15 expression to enhances T cell proliferation. Human T cells were transduced with a measles virus (MV) pseudotyped lentiviral vector redirected specifically against human CD4, encoding the NGFR reporter alone (“no IL15 construct”), soluble IL15 along with the reporter, or membrane-bound IL15 along with the reporter. Overview is shown in FIG. 1A. Transduction is shown in FIG. 1B, where the transgene is detected using a co-expressed reporter molecule (tNGFR). Quantification of soluble IL15 is depicted in FIG. 1C. No additional cytokine or stimulation signals were provided to the cells. Tcells express transgenic IL15 and IL15Ra-IL15. Cell number is shown in FIG. 1D, with superior expansion of the IL15+IL15R (mbIL15) cells. FIG. 1E shows that resting T cells transduced with the MV pseudotyped lentivector conferring IL15 or IL15R+IL15 expression expand, whereas resting T cells transduced with the standard VSVG pseudotyped lentivector do not expand.

FIGS. 2A-2D: Transgenic TPO-R enables controllable T cell expansion. T cells were transduced with the reporter NGFR, or with reporter+the thrombopoietin receptor (TPOR) and stimulated with eltrombopag, a specific small molecule agonist of TPOR, in vitro. FIG. 2A: Cell number is shown to increase only in the presence of the TPOR agonist. FIG. 2B: After 12 days of treatment with eltrombopag, there is specific expansion of MV-transduced CD4+ cells that express the transgene. FIG. 2C: All cells that express the reporter (tNGFR) also express the TPO receptor. FIG. 2D: Unactivated (resting) T cells transduced with the MV pseudotyped lentivector expand selectively in response to thrombopoietin (the natural ligand of TPOR) or the small molecule agonist eltrombopag.

FIG. 3: T cells transduced to express (i) the thrombopoietin receptor (TPOR) or (ii) the GCF receptor with a deletion at aa 715 (GCSFRd715) are active and can drive T cell proliferation via the non-canonical JAK2 pathway. T cells expressing GCSFRd715, TPOR, or the truncated NGFR as a control were stimulated for 7 days with IL-2/IL-7/IL-15 (all at 5 ng/ml; denoted as CK; positive control condition), 0.1 μg/ml recombinant thrombopoietin (TPO), 0.1 μg/ml TPO agonist eltrombopag (EP), or 0.1 μg/ml GCSF. Cell number (top) or percent transgenic cells (bottom) were quantified using flow cytometry at 7 days.

FIG. 4: Exemplary flow cytometry plots showing that exposure of transgenic cytokine receptor transduced cells to the cognate cytokine (i.e. GCSFRd715 to GCSF; TPOR to TPO or EP) leads to enrichment of CAR expressing cells at 7 days. Addition of GCSF enriched the GCSRFd715 transgenic cell fraction from 15.6% to 64.9% (top panel). Addition of TPO enriched the TPOR fraction from 12.9% to 82% and addition of EP enriched the TPOR fraction from 12.9% to 55.9% (bottom panel).

FIG. 5: TPOR stimulation leads to activation of STAT3 and STAT5. Healthy donor Tcells expressing TPOR or untransduced control T cells were stimulated for 24 hours with 5 ng/ml IL-2/IL-7/IL-15 (CK), 0.1 μg/ml TPO or 0.1 μg/ml Eltrombopag (EP). Following stimulation cells were fixed and the phosphorylation of STAT3 and STAT5 quantified using flow cytometry. These results show that TPO or EP lead to the expected STAT5 and STAT3 phosphorylation.

FIG. 6: Comparison of CAR20-TPOR vs. CAR20-GCSFRd715. Healthy donor CD8 Tcells expressing CAR20, CAR20-TPOR or CAR20-GCSFRd715 were incubated with CD20+ Raji tumor cells and target cell lysis was quantified by luminescence. These results show that expression of the transgenic cytokine receptors does not diminish the activity of CART-20 cells.

### DETAILED DESCRIPTION

The present invention provides compositions and methods for controllable stimulation of genetically engineered lymphocytes (e.g. CAR T cells) both in vivo and ex vivo. Compositions and methods of treatment are also provided.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2nd edition).

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

The term “ectopic” means “out of place.” For example, ectopic expression is an abnormal gene expression in a cell type, tissue type, or developmental stage in which the gene is not usually expressed. For example, a receptor that is not normally expressed in a particular cell would be an ectopic receptor.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “pseudotyped” or “pseudotyped viral particle”, as used herein, refers to a viral particle bearing envelope glycoproteins derived from other viruses having envelopes or a viral vector encoding envelope glycoproteins from a virus that is different from the parental virus. The host range of the vector particles can thus be expanded or altered depending on the type of cell surface receptor used by the glycoprotein. For example, a HIV lentiviral vector can have the HIV envelope glycoprotein be replaced with the VSV envelope glycoprotein. This is just one non-limiting example and other envelop glycoproteins can be used, such as the envelope glycoprotein of the Nipah virus. Therefore, in some embodiments, the viral particle is encoded by a lentivirus that encodes the Nipah viral envelope glycoprotein. In some embodiments, the Nipah viral envelope glycoprotein is glycoprotein F. In some embodiments, the Nipah viral envelope glycoprotein is glycoprotein G. In some embodiments, the pseudotyped viral vector encodes both the Nipah viral glycoprotein F and glycoprotein G. In some embodiments, the pseudotyped viral particle expresses one or both of the Nipah viral glycoprotein F and glycoprotein G.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A“transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

As used herein, the term “variant” when used in conjunction to an amino acid sequence refers to a sequence that is at least, or about, 85%, 90%, 91%, 92%, 93%, 94%, 95%, 96%, 97%, 98%, or 99% identical to the reference sequence. In some embodiments, the variant comprises 1, 2, 3, 4, 5, 6, 7, 8, 9, or 10 substitutions. In some embodiments, the substitution is a conservative substitution.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Methods of Expanding Cells and Controlling Cell Expansion

Provided herein are methods for expanding immune cells or precursors thereof, e.g., Tcells, e.g., CAR T cells, and methods of controlling expansion of cells e.g., T cells, e.g., CAR Tcells.

In one aspect, the invention provides methods for controlling cell expansion e.g., T cell expansion, e.g., CAR T cell expansion, through use of a modified cell (e.g., T cell, e.g., CAR T cell) comprising an ectopic/endogeneous cytokine receptor. Such cytokine receptors include but are not limited to erythropoietin receptor (Epo-R), thrombopoietin receptor (TPO-R), and granulocyte colony-stimulating factor (GCSF) receptor (G-CSFR). Expansion of the cells can be controlled both in vitro and in vivo by administering a cognate cytokine (e.g., erythropoietin, thrombopoietin, GCSF) to stimulate the cells, or a cognate inhibitor to inhibit the cells.

Cytokine receptors exist on the cell surface in an inactive state bound to JAKs via their cytosolic domains. The binding of a specific ligand induces a conformational change in the preformed dimer, leading to tyrosine phosphorylation and cross-activation of JAKs, which phosphorylate intracellular receptor tyrosine residues. In turn, the phosphorylated residues attract signaling adaptor proteins that recognize specific tyrosine phosphorylated sequences. Various adaptor proteins become substrates of JAKs, triggering signaling cascades. Cytokine receptors are linked to the STAT, Ras-MAPK, and phosphatidylinositol-3′-kinase (PI3K)-AKT pathways, which converge at the nucleus and regulate gene expression. In an exemplary embodiment, a ligand-activated receptor is transient and induces anti-apoptotic, proliferative, and differentiation signals. In another exemplary embodiment, an unliganded receptor that is constitutively active e.g. because of the attachment of JAK2V617F, a constitutively active JAK, is therefore recapitulating the cytokine-induced pathway, although in a persistent manner. Novel signaling molecules become substrates of activated JAK, initiating novel interactions that change gene expression and/or induce novel epigenetic events (Vainchenker & Constantinescu, Oncogene 2012; 32:2601-2013).

In one aspect, the invention provides a method of controlling T cell expansion in subject comprising administering to the subject, a modified T cell comprising an ectopic Granulocyte Colony Stimulating Factor receptor (G-CSFR). The method further comprises administering Granulocyte Colony Stimulating Factor (G-CSF) to the subject to stimulate expansion of the T cell, and/or administering an inhibitor of G-CSF, JAK 2, or JAK1/2 to the subject to inhibit expansion of the T cell.

In certain embodiments, the G-CSFR is an alternatively spliced class IV G-CSFR (such as described in Mehta et al. (2014) Leukemia. 28(5):1041-51). Any variant or form of G-CSF known in the art can be used with the methods disclosed herein. In certain embodiments, the G-CSF is recombinant human G-CSF (rhG-CSF).

Any variant or form of G-CSF inhibitor, JAK2 inhibitor, or JAK1/2 inhibitor known in the art can be used with the methods disclosed herein. JAK2-specific inhibitors include but are not limited to NVP-BSK805, ruxolitinib, fedratinib, gandotinib, lestaurtinib, and pacritinib. JAK1/2 inhibitors include but are not limited to INCB018424, ruxolitinib, baricitinib, and memolitinib. JAK1 inhibitors include but are not limited to oclacitinib, upadacitinib, and abrocitinib. JAK3 inhibitors include but are not limited to tofacitinib and peficitinib. Inhibitors in the form of antibodies, drugs, small molecules, siRNA, miRNA, CRISPR, and the like can also be used.

In another aspect, the invention includes methods for expanding immune cells or precursors thereof, e.g., T cells, e.g., a CAR T cells, comprising introducing a nucleic acid encoding a cytokine into the cell.

In certain embodiments, the cytokine comprises a membrane bound version of IL15 (mbIL15). IL15 is a homeostatic cytokine that is required for the maintenance of CD8+ memory T cells and NK cells, and promotes anti-tumor activity. IL15 signals through the IL15 receptor which is composed of a specific a chain (herein referred to as IL15Ralpha, IL15Rα, or IL15Ra interchangeably) along with the shared IL2Rβ (also referred to as IL2RB, IL2Rbeta, or IL2Rb) and common gamma chain (also referred to as IL2Rg, IL2Rgamma, or IL2Rγ). In certain embodiments, the mbIL15 comprises a fusion protein comprising IL15 linked to IL115Ra via a flexible linker. Fusing IL15 directly to IL115Ra via a flexible linker promotes IL15 signaling to the expressing cell (see e.g. US 2016/0158285 A1). In certain embodiments, the mbIL15 comprises an amino acid sequence at least about 85%, 90%, 91%, 92%, 93%, 94%, 95%, 96%, 97%, 98% or 99% identical to SEQ ID NO: 1 (SEQ ID NO: 6 of US 2016/0158285 A1,incorporated herein by reference). Modified cells comprising mbIL15 can proliferate and/or persist with little or no ex vivo culture with activating and propagating cells or artificial antigen presenting cells (aAPCs) due the simulation provided by the cytokine expression. Such modified cells (e.g. CAR T cells comprising mbIL15) can proliferate in vivo even when large amounts of antigen recognized by the CAR is not present (e.g., as in the case of a cancer patient in remission or a patient with minimal residual disease).

In certain embodiments, the nucleic acid comprises a measles virus (MV) pseudotyped lentiviral vector encoding a cytokine. MV pseudotyped lentiviral vectors are described in detail in PCT/US2021/021904, which is incorporated by reference in its entirety herein. Briefly, a vector (e.g. a lentiviral vector) can be pseudotyped with a morbillivirus, such as a measles virus, glycoprotein and/or a Nipah virus glycoprotein (e.g., F protein or H protein of a morbillivirus or F protein or G protein of a Nipah virus). In certain embodiments, the lentivirus is pseudotyped with a measles virus (MV) hemagglutinin (HA) protein and an MV fusion (F) protein, wherein the MV-HA protein or the MV-F protein comprises a mutated binding domain compared to its naturally occurring binding domain. In certain embodiments, the lentivirus is pseudotyped with a Nipah virus fusion protein (F protein), or a Nipah virus attachment protein (G protein). In certain embodiments, the measles virus (MV) pseudotyped lentiviral vector comprises a chimeric gag protein (e.g., xHIV gag protein). In certain embodiments, the MV pseudotyped lentiviral vector encodes a membrane bound version of IL15 (mbIL15). In certain embodiments, the mbIL15 comprises a fusion protein comprising IL15 linked to a via a flexible linker. In certain embodiments, the MV pseudotyped lentiviral vector encoding mbIL15 encodes an amino acid sequence set forth in SEQ ID NO: 1.

In certain embodiments, the immune cell or precursor cell thereof is a T cell. In certain embodiments, the T cell is human T cell. In certain embodiments, T cell is an autologous T cell.

In one aspect, the invention provides a method of expanding CAR T cells. The method comprises introducing into the CAR T cell a measles virus (MV) pseudotyped lentiviral vector encoding a membrane bound version of IL15 (mbIL15). In certain embodiments, the mbIL15 comprises a fusion protein comprising IL15 linked to IL15Rα via a flexible linker. In certain embodiments, the expansion method generates a population of cells that is 1-, 2-, 5-, or 10-fold, 20-fold, 50-fold, 100-fold, or greater than 100-fold higher in cell number compared to a method that does not comprise a measles virus (MV) pseudotyped lentiviral vector encoding mbIL15.

In another aspect, the invention provides a method of generating a population of cells for use in an immunotherapy. The method comprises introducing into an immune cell or precursor cell thereof a measles virus (MV) pseudotyped lentiviral vector encoding a membrane bound version of IL15 (mbIL15) and a chimeric antigen receptor (CAR). In certain embodiments, the mbIL15 comprises a fusion protein comprising IL15 linked to IL15Rα via a flexible linker. In certain embodiments, the method generates a population of cells that is 1-, 2-, 5-, 10-fold, 20-fold, 50-fold, 100-fold, or greater than 100-fold higher in cell number compared to a method that does not comprise a measles virus (MV) pseudotyped lentiviral vector encoding mbIL15.

In certain embodiments, the immune cell or precursor cell thereof is a T cell. In certain embodiments, the T cell is human T cell. In certain embodiments, T cell is an autologous T cell.

In certain embodiments, the method comprises a) obtaining a sample of cells (e.g., immune cells or precursors thereof, e.g., T cells or precursors thereof) from a subject, b) introducing a measles virus (MV) pseudotyped lentiviral vector encoding a chimeric antigen receptor (CAR) and a membrane bound version of IL15 (mbIL15) into the cells, generating a population of modified CAR T cells comprising expressed mbIL15, c) optionally, culturing the modified cells ex vivo in order to expand the cells, or alternatively, administering the cells to the subject wherein the cells are expanded in vivo. The modified cells can be administered to the subject before or after expansion in order to treat a disease or disorder, e.g., cancer.

In certain embodiments, the modified cells are cultured ex vivo in a medium that promotes proliferation of the cells. In certain embodiments, the modified cells are cultured ex vivo for less than 21 days, such as for less than 18, 17, 16, 15, 14, 13, 12, 11, 10, 9, 8, 7, 6, 5, 4, 3,2 days or less. In certain embodiments, the modified cells are cultured ex vivo no more than 3 to 5 days. In still certain embodiments, the entire method (i.e., obtaining cell samples to administering modified cells) is completed in no more that 21, 20, 19, 18, 17, 16, 15, 14, 13, 12, 11, 10, 9, 8, 7, 6, or 5 days.

The cells can be further activated and expanded using methods as described, for example, in U.S. Pat. Nos. 5,199,942, 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the Tcells of the invention may be expanded by contact with a surface having attached thereto an agent that stimulates a CD3/TCR complex associated signal and a ligand that stimulates a co-stimulatory molecule on the surface of the T cells. In particular, T cell populations may be stimulated by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the cells expand in the range of about 20 fold to about 50 fold.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15,(Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the T cells may include an agent that can co-stimulate the Tcells. For example, an agent that can stimulate CD3 is an antibody to CD3, and an agent that can stimulate CD28 is an antibody to CD28. A cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the Tcells expand in the range of about 20 fold to about 50 fold, or more. In one embodiment, human T cells are expanded via anti-CD3 antibody coated KT64.86 artificial antigen presenting cells (aAPCs). Methods for expanding and activating T cells can be found in U.S. Pat. Nos. 7,754,482, 8,722,400, and 9,555,105, contents of which are incorporated herein in their entirety.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

### C. Modified Immune Cells

The present invention provides modified immune cells or precursors thereof (e.g., Tcells) for use in immunotherapy (e.g. CAR T cells). In one aspect, the invention provides a modified immune cell or precursor cell thereof (e.g., T cell) comprising a chimeric antigen receptor (CAR) and an ectopic/exogeneous G-CSF receptor (G-CSFR). In certain embodiments, the G-CSFR is an alternatively spliced class IV G-CSFR.

In certain embodiments, the immune cell or precursor cell thereof is a T cell. In certain embodiments, the T cell is a human T cell. In certain embodiments, the cell is an autologous cell (e.g. an autologous T cell).

The modified cells can comprise any chimeric antigen receptor (CAR), which are described in detail elsewhere herein.

Thus, provided are cells, compositions and methods that enhance immune cell, such as T cell, function in adoptive cell therapy, including those offering improved efficacy, such as by increasing activity and potency of administered genetically engineered cells, while maintaining persistence or exposure to the transferred cells over time. In some embodiments, the modified cells, exhibit increased expansion and/or persistence when administered in vivo to a subject, as compared to certain available methods. In some embodiments, the provided immune cells exhibit increased persistence when administered in vivo to a subject. In some embodiments, the persistence of genetically engineered immune cells, in the subject upon administration is greater as compared to that which would be achieved by alternative methods, such as those involving administration of cells genetically engineered by methods in which T cells do not encode an ectopic/exogeneous G-CSF receptor. In some embodiments, the persistence is increased at least or about at least 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In some embodiments, the degree or extent of persistence of administered cells can be detected or quantified after administration to a subject. For example, in some aspects, quantitative PCR (qPCR) is used to assess the quantity of cells expressing the exogenous receptor (e.g., G-CSFR and/or CAR) in the blood or serum or organ or tissue (e.g., disease site) of the subject. In some aspects, persistence is quantified as copies of DNA or plasmid encoding the exogenous receptor per microgram of DNA, or as the number of receptor-expressing cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the exogenous receptor (e.g. exogenous G-CSFR and/or CAR) can be used to distinguish the administered cells from endogenous cells in a subject.

### D. Chimeric Antigen Receptors

The present invention provides compositions and methods for modified immune cells or precursors thereof, e.g., modified T cells, comprising a chimeric antigen receptor (CAR). Thus, in some embodiments, the immune cell has been genetically modified to express the CAR. CARs of the present invention comprise an antigen binding domain, a transmembrane domain, and an intracellular domain.

The antigen binding domain may be operably linked to another domain of the CAR, such as the transmembrane domain or the intracellular domain, both described elsewhere herein, for expression in the cell. In one embodiment, a first nucleic acid sequence encoding the antigen binding domain is operably linked to a second nucleic acid encoding a transmembrane domain, and further operably linked to a third a nucleic acid sequence encoding an intracellular domain.

The antigen binding domains described herein can be combined with any of the transmembrane domains described herein, any of the intracellular domains or cytoplasmic domains described herein, or any of the other domains described herein that may be included in a CAR of the present invention. A subject CAR of the present invention may also include a hinge domain as described herein. A subject CAR of the present invention may also include a spacer domain as described herein. In some embodiments, each of the antigen binding domain, transmembrane domain, and intracellular domain is separated by a linker.

### Antigen Binding Domain

The antigen binding domain of a CAR is an extracellular region of the CAR for binding to a specific target antigen including proteins, carbohydrates, and glycolipids. In some embodiments, the CAR comprises affinity to a target antigen on a target cell. The target antigen may include any type of protein, or epitope thereof, associated with the target cell. For example, the CAR may comprise affinity to a target antigen on a target cell that indicates a particular disease state of the target cell.

In one embodiment, the target cell antigen is a tumor associated antigen (TAA). Examples of tumor associated antigens (TAAs), include but are not limited to, differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4,MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS. In a preferred embodiment, the antigen binding domain of the CAR targets an antigen that includes but is not limited to CD19, CD20, CD22, ROR1, Mesothelin, CD33/IL3Ra, c-Met, PSMA, PSCA, Glycolipid F77, EGFRvIII, GD-2, MY-ESO-1 TCR, MAGE A3 TCR, and the like.

Depending on the desired antigen to be targeted, the CAR of the invention can be engineered to include the appropriate antigen binding domain that is specific to the desired antigen target. For example, if CD19 is the desired antigen that is to be targeted, an antibody for CD19 can be used as the antigen bind moiety for incorporation into the CAR of the invention.

In one embodiment, the target cell antigen is CD19. As such, in one embodiment, a CAR of the present disclosure has affinity for CD19 on a target cell. This should not be construed as limiting in any way, as a CAR having affinity for any target antigen is suitable for use in a composition or method of the present invention.

As described herein, a CAR of the present disclosure having affinity for a specific target antigen on a target cell may comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin. In one embodiment, a CAR of the present disclosure having affinity for CD19 on a target cell may comprise a CD19 binding domain.

In some embodiments, a CAR of the present disclosure may have affinity for one or more target antigens on one or more target cells. In some embodiments, a CAR may have affinity for one or more target antigens on a target cell. In such embodiments, the CAR is a bispecific CAR, or a multispecific CAR. In some embodiments, the CAR comprises one or more target-specific binding domains that confer affinity for one or more target antigens. In some embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through an oligo- or polypeptide linker, an Fc hinge region, or a membrane hinge region.

In some embodiments, the antigen binding domain is selected from the group consisting of an antibody, an antigen binding fragment (Fab), and a single-chain variable fragment (scFv). In some embodiments, a CD19 binding domain of the present invention is selected from the group consisting of a CD19-specific antibody, a CD19-specific Fab, and a CD19-specific scFv. In one embodiment, a CD19 binding domain is a CD19-specific antibody. In one embodiment, a CD19 binding domain is a CD19-specific Fab. In one embodiment, a CD19 binding domain is a CD19-specific scFv.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. In some embodiments, the antigen binding domain portion comprises a mammalian antibody or a fragment thereof. The choice of antigen binding domain may depend upon the type and number of antigens that are present on the surface of a target cell.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen binding domain (e.g., PSCA binding domain) comprises an scFv having the configuration from N-terminus to C-terminus, VH-linker-VL. In some embodiments, the antigen binding domain comprises an scFv having the configuration from N-terminus to C-terminus, VL-linker-VH. Those of skill in the art would be able to select the appropriate configuration for use in the present invention.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO:15), (GGGS)ₙ (SEQ ID NO:16), and (GGGGS)ₙ (SEQ ID NO:17), where n represents an integer of at least 1. Exemplary linker sequences can comprise amino acid sequences including, without limitation, GGSG (SEQ ID NO:18), GGSGG (SEQ ID NO:19), GSGSG (SEQ ID NO:20), GSGGG (SEQ ID NO:21), GGGSG (SEQ ID NO: 22), GSSSG (SEQ ID NO:23), GGGGS (SEQ ID NO:24), GGGGSGGGGSGGGGS (SEQ ID NO:25) and the like. Those of skill in the art would be able to select the appropriate linker sequence for use in the present invention. In one embodiment, an antigen binding domain of the present invention comprises a heavy chain variable region (VH) and a light chain variable region (VL), wherein the VH and VL is separated by the linker sequence having the amino acid sequence GGGGSGGGGSGGGGS (SEQ ID NO:25), which may be encoded by the nucleic acid sequence GGTGGCGGTGGCTCGGGCGGTGGTGGGTCGGGTGGCGGCGGATCT (SEQ ID NO:26).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 August 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and alight (L) chain linked by an S—S bond for binding an antigen and where the remaining H chain portions are linked together. A “F(ab′)2” fragment can be split into two individual Fab′ fragments.

In some embodiments, the antigen binding domain may be derived from the same species in which the CAR will ultimately be used. For example, for use in humans, the antigen binding domain of the CAR may comprise a human antibody or a fragment thereof. In some embodiments, the antigen binding domain may be derived from a different species in which the CAR will ultimately be used. For example, for use in humans, the antigen binding domain of the CAR may comprise a murine antibody or a fragment thereof.

### Transmembrane Domain

CARs of the present invention may comprise a transmembrane domain that connects the antigen binding domain of the CAR to the intracellular domain of the CAR. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In some embodiments, the transmembrane domain is naturally associated with one or more of the domains in the CAR. In some embodiments, the transmembrane domain can be selected or modified by one or more amino acid substitutions to avoid binding of such domains to the transmembrane domains of the same or different surface membrane proteins, to minimize interactions with other members of the receptor complex.

The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane domain of particular use in this invention include, without limitation, transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22,CD33, CD37, CD64, CD80, CD86, CD134 (OX-40), CD137 (4-1BB), CD154 (CD40L), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen binding domains described herein, any of the intracellular domains described herein, or any of the other domains described herein that may be included in a subject CAR.

In some embodiments, the transmembrane domain further comprises a hinge region. A subject CAR of the present invention may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, a subject CAR of the present disclosure includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell (Hudecek et al., supra). The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa. In some embodiments, the hinge region can have a length of greater than 5 aa, greater than 10 aa, greater than 15 aa, greater than 20 aa, greater than 25 aa, greater than 30 aa, greater than 35 aa, greater than 40 aa, greater than 45 aa, greater than 50 aa, greater than 55 aa, or more.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge regions can have a length of greater than 20 amino acids (e.g., 30, 40, 50, 60 or more amino acids).

For example, hinge regions include glycine polymers (G)ₙ, glycine-serine polymers (including, for example, (GS)ₙ, (GSGGS)ₙ (SEQ ID NO:15) and (GGGS)ₙ (SEQ ID NO:16), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). Exemplary hinge regions can comprise amino acid sequences including, but not limited to, GGSG (SEQ ID NO:18), GGSGG (SEQ ID NO:19), GSGSG (SEQ ID NO:20), GSGGG (SEQ ID NO:21), GGGSG (SEQ ID NO:22), GSSSG (SEQ ID NO:23), and the like.

In some embodiments, the hinge region is an immunoglobulin heavy chain hinge region. Immunoglobulin hinge region amino acid sequences are known in the art; see, e.g., Tan et al., Proc. Natl. Acad. Sci. USA (1990) 87(1):162-166; and Huck et al., Nucleic Acids Res. (1986) 14(4): 1779-1789. As non-limiting examples, an immunoglobulin hinge region can include one of the following amino acid sequences: DKTHT (SEQ ID NO:27); CPPC (SEQ ID NO:28); CPEPKSCDTPPPCPR (SEQ ID NO:29) (see, e.g., Glaser et al., J. Biol. Chem. (2005) 280:41494-41503); ELKTPLGDTTHT (SEQ ID NO:30); KSCDKTHTCP (SEQ ID NO:31); KCCVDCP (SEQ ID NO:32); KYGPPCP (SEQ ID NO:33); EPKSCDKTHTCPPCP (SEQ ID NO:34) (human IgG1 hinge); ERKCCVECPPCP (SEQ ID NO:35) (human IgG2 hinge); ELKTPLGDTTHTCPRCP (SEQ ID NO:36) (human IgG3 hinge); SPNMVPHAHHAQ (SEQ ID NO:37) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO:38); see, e.g., Yan et al., J. Biol. Chem. (2012) 287: 5891-5897. In one embodiment, the hinge region can comprise an amino acid sequence derived from human CD8, or a variant thereof.

### Intracellular Signaling Domain

A subject CAR of the present invention also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcyRIII, FcsRI, cytoplasmic tails of Fc receptors, an immunoreceptor tyrosine-based activation motif (ITAM) bearing cytoplasmic receptors, and combinations thereof.

In one embodiment, the intracellular signaling domain of the CAR includes any portion of one or more co-stimulatory molecules, such as at least one signaling domain from CD2, CD3,CD8, CD27, CD28, ICOS, 4-1BB, PD-1, any derivative or variant thereof, any synthetic sequence thereof that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127,CD160, CD19, CD4, CD8alpha, CD8beta, IL2R beta, IL2R gamma, IL7R alpha, ITGA4, VLA1,CD49a, ITGA4, IA4, CD49D, ITGA6, VLA-6, CD49f, ITGAD, CD11d, ITGAE, CD103,ITGAL, CD11a, LFA-1, ITGAM, CDlib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84,CD96 (Tactile), CEACAMI, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8,TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a subject CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a subject CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs.

In some embodiments, intracellular signaling domains includes the signaling domains of human immunoglobulin receptors that contain immunoreceptor tyrosine based activation motifs (ITAMs) such as, but not limited to, FcgammaRI, FcgammaRIIA, FcgammaRIIC, FcgammaRIIIA, FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceRl gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in an FN3 CAR of the present disclosure includes a DAP10/CD28 type signaling chain. In one embodiment, an intracellular signaling domain suitable for use in an FN3 CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the antigen binding domains described herein, any of the transmembrane domains described herein, or any of the other domains described herein that may be included in the CAR.

### E. Methods of Treatment

The modified cells (e.g., T cells) described herein may be included in a composition for immunotherapy. The composition may include a pharmaceutical composition and further include a pharmaceutically acceptable carrier. A therapeutically effective amount of the pharmaceutical composition comprising the modified T cells may be administered.

In one aspect, the invention includes a method for adoptive cell transfer therapy comprising administering to a subject in need thereof a modified T cell of the present invention. In another aspect, the invention includes a method of treating a disease or condition in a subject comprising administering to a subject in need thereof a population of modified T cells.

In another aspect, the invention includes a method of treating a disease or disorder in a subject in need thereof, comprising administering to the subject a modified T cell comprising a CAR and an ectopic/exogenous G-CSFR. In certain embodiments, the modification of the T cell is performed in vivo in the subject via a vector (e.g. a lentiviral vector). In certain embodiments, the disease or disorder is cancer. In certain embodiments, the method further comprises administering G-CSF to the subject to stimulate expansion of the T cell. In certain embodiments, the method further comprises administering an inhibitor of G-CSF, JAK 2, or JAK1/2 to the subject to inhibit expansion of the T cell. In certain embodiments, the G-CSFR is an alternatively spliced class IV G-CSFR. In certain embodiments, the G-CSF is recombinant rhG-CSF. In certain embodiments, the method is used to prevent chemotherapy-associated neutropenia.

In another aspect, the invention includes a method treating a disease or disorder (e.g. cancer) in a subject in need thereof. The method comprises administering to the subject a modified an immune cell or precursor cell thereof generated by introducing a measles virus (MV) pseudotyped lentiviral vector encoding a membrane bound version of IL15 (mbIL15) and a chimeric antigen receptor (CAR) into the cell. In some embodiments, the introduction of the lentiviral vector occurs in vivo in the subject. In this way, the generation of the modified immune cell or precursor cell thereof occurs within the subject.

Methods for administration of immune cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The modified immune cells of the present invention can be administered to an animal, preferably a mammal, even more preferably a human, to treat a cancer. In addition, the cells of the present invention can be used for the treatment of any condition related to a cancer, especially a cell-mediated immune response against a tumor cell(s), where it is desirable to treat or alleviate the disease. The types of cancers to be treated with the modified cells or pharmaceutical compositions of the invention include, carcinoma, blastoma, and sarcoma, and certain leukemia or lymphoid malignancies, benign and malignant tumors, and malignancies e.g., sarcomas, carcinomas, and melanomas. Other exemplary cancers include but are not limited breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included. In one embodiment, the cancer is a solid tumor or a hematological tumor. In one embodiment, the cancer is a carcinoma. In one embodiment, the cancer is a sarcoma. In one embodiment, the cancer is a leukemia. In one embodiment the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

In certain exemplary embodiments, the modified immune cells of the invention are used to treat a myeloma, or a condition related to myeloma. Examples of myeloma or conditions related thereto include, without limitation, light chain myeloma, non-secretory myeloma, monoclonal gamopathy of undetermined significance (MGUS), plasmacytoma (e.g., solitary, multiple solitary, extramedullary plasmacytoma), amyloidosis, and multiple myeloma. In one embodiment, a method of the present disclosure is used to treat multiple myeloma. In one embodiment, a method of the present disclosure is used to treat refractory myeloma. In one embodiment, a method of the present disclosure is used to treat relapsed myeloma.

In certain exemplary embodiments, the modified immune cells of the invention are used to treat a melanoma, or a condition related to melanoma. Examples of melanoma or conditions related thereto include, without limitation, superficial spreading melanoma, nodular melanoma, lentigo maligna melanoma, acral lentiginous melanoma, amelanotic melanoma, or melanoma of the skin (e.g., cutaneous, eye, vulva, vagina, rectum melanoma). In one embodiment, a method of the present disclosure is used to treat cutaneous melanoma. In one embodiment, a method of the present disclosure is used to treat refractory melanoma. In one embodiment, a method of the present disclosure is used to treat relapsed melanoma.

In yet other exemplary embodiments, the modified immune cells of the invention are used to treat a sarcoma, or a condition related to sarcoma. Examples of sarcoma or conditions related thereto include, without limitation, angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, and synovial sarcoma. In one embodiment, a method of the present disclosure is used to treat synovial sarcoma. In one embodiment, a method of the present disclosure is used to treat liposarcoma such as myxoid/round cell liposarcoma, differentiated/dedifferentiated liposarcoma, and pleomorphic liposarcoma. In one embodiment, a method of the present disclosure is used to treat myxoid/round cell liposarcoma. In one embodiment, a method of the present disclosure is used to treat a refractory sarcoma. In one embodiment, a method of the present disclosure is used to treat a relapsed sarcoma.

The cells of the invention to be administered may be autologous, with respect to the subject undergoing therapy.

The administration of the cells or vectors of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4⁺ to CD8⁺ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ Tcells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4⁺ cells and/or a desired dose of CD8⁺ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ Tcells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1,such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In certain embodiments, the modified cells of the invention (e.g., a modified cell comprising a CAR) may be administered to a subject in combination with an immune checkpoint antibody (e.g., an anti-PD1, anti-CTLA-4, or anti-PDL1 antibody). For example, the modified cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-L1 antibodies include, but are not limited to, BMS-936559, MPDL3280A(TECENTRIQ®, Atezolizumab), and MEDI4736 (Durvalumab, Imfinzi). In certain embodiments, the modified cell may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the modified cell comprising the CAR. In certain embodiments, combination treatment comprising an immune checkpoint modulator may increase the therapeutic efficacy of a therapy comprising a modified cell of the present invention.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to CAR T cell therapy. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine. In some embodiments, a specific dosage regimen of the present disclosure does not include a lymphodepletion step prior to the administration of the modified T cells.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

Dosing of lymphodepletion chemotherapy may be scheduled on Days −6 to −4 (with a −1 day window, i.e., dosing on Days −7 to −5) relative to T cell (e.g., CAR-T, TCR-T, a modified T cell, etc.) infusion on Day 0.

In an exemplary embodiment, for a subject having cancer, the subject receives lymphodepleting chemotherapy including 300 mg/m² of cyclophosphamide by intravenous infusion 3 days prior to administration of the modified T cells. In an exemplary embodiment, fora subject having cancer, the subject receives lymphodepleting chemotherapy including 300 mg/m² of cyclophosphamide by intravenous infusion for 3 days prior to administration of the modified T cells.

In an exemplary embodiment, for a subject having cancer, the subject receives lymphodepleting chemotherapy including fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including fludarabine at a dose of 30 mg/m² for 3 days.

In an exemplary embodiment, for a subject having cancer, the subject receives lymphodepleting chemotherapy including cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including cyclophosphamide at a dose of about 300 mg/m²/day, and fludarabine at a dose of 30 mg/m² for 3 days.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the invention provides for, following the diagnosis of CRS, appropriate CRS management strategies to mitigate the physiological symptoms of uncontrolled inflammation without dampening the antitumor efficacy of the engineered cells (e.g., CAR Tcells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019)Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

As such, the modified immune cells of the present invention when used in a method of treatment as described herein, enhances the ability of the modified immune cells in carrying out their function. Accordingly, the present invention provides a method for enhancing a function of a modified immune cell for use in a method of treatment as described herein.

In one aspect, the invention includes a method of treating cancer in a subject in need thereof, comprising administering to the subject any one of the modified immune or precursor cells disclosed herein. Yet another aspect of the invention includes a method of treating cancer in a subject in need thereof, comprising administering to the subject a modified immune or precursor cell generated by any one of the methods disclosed herein.

### F. Methods of Producing Genetically Modified Immune Cells

The present disclosure provides methods for producing or generating a modified immune cell or precursor thereof (e.g., a T cell) of the invention, e.g., for adoptive immunotherapy. The cells generally are engineered by introducing one or more genetically engineered nucleic acids encoding the exogenous receptor(s) (e.g., CAR and/or cytokine receptor).

In one aspect, the invention includes a method of generating a population of cells for use as an immunotherapy. The method comprises introducing into an immune cell or precursor cell thereof, a measles virus (MV) pseudotyped lentiviral vector encoding a membrane bound version of IL15 (mbIL15) and a chimeric antigen receptor (CAR). In certain embodiments, the mbIL15 comprises a fusion protein comprising IL15 linked to IL15Rα via a flexible linker. In certain embodiments, the introduction of the lentiviral vector is performed in vivo. The in vivo transduction can be used to introduce the nucleic acid molecule encoding the CAR and/or the mbIL15. In certain embodiments, the method generates a population of cells that is 1-, 2-, 5-, or 10-fold, 20-fold, 50-fold, 100-fold, or greater than 100-fold higher in cell number compared to a method that does not comprise a measles virus (MV) pseudotyped lentiviral vector encoding mbIL15.

In certain embodiments, the immune cell or precursor cell thereof is a T cell. In certain embodiments, the T cell is human T cell. In certain embodiments, T cell is an autologous T cell.

In some embodiments, the exogenous receptor (e.g., CAR and/or cytokine receptor) is introduced into a cell by an expression vector. Expression vectors comprising a nucleic acid sequence encoding a CAR and/or cytokine receptor of the present invention are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggybak, and Integrases such as Phi31. Some other suitable expression vectors include Herpes simplex virus (HSV) and retrovirus expression vectors.

In certain embodiments, the nucleic acid encoding an exogenous CAR or cytokine receptor is introduced into the cell via viral transduction. In certain embodiments, the viral transduction comprises contacting the immune or precursor cell with a viral vector comprising the nucleic acid encoding an exogenous CAR and/or cytokine receptor. In certain embodiments, the viral vector is an adeno-associated viral (AAV) vector. In certain embodiments, the AAV vector comprises a 5′ ITR and a 3′ITR derived from AAV6. In certain embodiments, the AAV vector comprises a Woodchuck Hepatitis Virus post-transcriptional regulatory element (WPRE). In certain embodiments, the AAV vector comprises a polyadenylation (polyA) sequence. In certain embodiments, the polyA sequence is a bovine growth hormone (BGH) polyA sequence.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the TCR and/or CAR in the host cell. In some embodiments, the adenovirus genome is a 36 kb, linear, double stranded DNA, where a foreign DNA sequence (e.g., a nucleic acid encoding an exogenous TCR and/or CAR) may be inserted to substitute large pieces of adenoviral DNA in order to make the expression vector of the present invention (see, e.g., Danthinne and Imperiale, Gene Therapy (2000) 7(20): 1707-1714).

Another expression vector is based on an adeno associated virus (AAV), which takes advantage of the adenovirus coupled systems. This AAV expression vector has a high frequency of integration into the host genome. It can infect nondividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retroviral vector is constructed by inserting a nucleic acid (e.g., a nucleic acid encoding an exogenous CAR and/or cytokine receptor) into the viral genome at certain locations to produce a virus that is replication defective. Though the retroviral vectors are able to infect a broad variety of cell types, integration and stable expression of the TCR and/or CAR requires the division of host cells.

Lentiviral vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the Human Immunodeficiency Viruses (HIV-1, HIV-2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are capable of infecting non-dividing cells and can be used for both in vivo and ex vivo gene transfer and expression, e.g., of a nucleic acid encoding a CAR (see, e.g., U.S. Pat. No. 5,994,136).

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell an immune cell or precursor thereof, e.g., a T cell, an NK cell, or an NKT cell.

The present invention also provides genetically engineered cells which include and stably express a CAR and/or cytokine receptor of the present disclosure. In some embodiments, the genetically engineered cells are genetically engineered T-lymphocytes (T cells), naive T cells (TN), memory T cells (for example, central memory T cells (TCM), effector memory cells (TEM)), natural killer cells (NK cells), and macrophages capable of giving rise to therapeutically relevant progeny. In certain embodiments, the genetically engineered cells are autologous cells.

Modified cells (e.g., comprising a CAR and/or cytokine receptor) may be produced by stably transfecting host cells with an expression vector including a nucleic acid of the present disclosure. Additional methods for generating a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a TCR and/or CAR of the present disclosure may be expanded ex vivo.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York. Chemical methods for introducing an expression vector into a host cell include colloidal dispersion systems, such as macromolecule complexes, nanocapsules, microspheres, beads, and lipid-based systems including oil-in-water emulsions, micelles, mixed micelles, and liposomes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform may be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). Compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, molecular biology assays well known to those of skill in the art, such as Southern and Northern blotting, RT-PCR and PCR; biochemistry assays, such as detecting the presence or absence of a particular peptide, e.g., by immunological means (ELISAs and Western blots) or by assays described herein to identify agents falling within the scope of the invention.

In one embodiment, the nucleic acids introduced into the host cell are RNA. In another embodiment, the RNA is mRNA that comprises in vitro transcribed RNA or synthetic RNA. The RNA may be produced by in vitro transcription using a polymerase chain reaction (PCR)-generated template. DNA of interest from any source can be directly converted by PCR into a template for in vitro mRNA synthesis using appropriate primers and RNA polymerase. The source of the DNA may be, for example, genomic DNA, plasmid DNA, phage DNA, cDNA, synthetic DNA sequence or any other appropriate source of DNA.

PCR may be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers may also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

In some embodiments, the RNA is electroporated into the cells, such as in vitro transcribed RNA. Any solutes suitable for cell electroporation, which can contain factors facilitating cellular permeability and viability such as sugars, peptides, lipids, proteins, antioxidants, and surfactants can be included.

In some embodiments, a nucleic acid encoding a TCR and/or CAR of the present disclosure will be RNA, e.g., in vitro synthesized RNA. Methods for in vitro synthesis of RNA are known in the art; any known method can be used to synthesize RNA comprising a sequence encoding a TCR and/or CAR. Methods for introducing RNA into a host cell are known in the art. See, e.g., Zhao et al. Cancer Res. (2010) 15: 9053. Introducing RNA comprising a nucleotide sequence encoding a TCR and/or CAR into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a TCR and/or CAR.

The disclosed methods can be applied to the modulation of T cell activity in basic research and therapy, in the fields of cancer, stem cells, acute and chronic infections, and autoimmune diseases, including the assessment of the ability of the genetically modified T cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the invention is that RNA transfection is essentially transient and a vector-free. An RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1,US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1.Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

In some embodiments, the immune cells (e.g. T cells) can be incubated or cultivated prior to, during and/or subsequent to introducing the nucleic acid molecule encoding the exogenous receptor (e.g., CAR and/or cytokine receptor). In some embodiments, the cells (e.g. T cells) can be incubated or cultivated prior to, during or subsequent to the introduction of the nucleic acid molecule encoding the exogenous receptor, such as prior to, during or subsequent to the transduction of the cells with a viral vector (e.g. lentiviral vector) encoding the exogenous receptor.

### G. Sources of Immune Cells

In certain embodiments, a source of immune cells is obtained from a subject (e.g. for ex vivo manipulation). Sources of cells manipulation may also include, e.g., autologous or heterologous donor blood, cord blood, or bone marrow. For example the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering/modifying them. In some embodiments, preparation of the engineered cells includes one or more culture and/or preparation steps. The cells for engineering/modifying as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from abiological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including Tcells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques. In some cases, such markers are those that are absent or expressed at relatively low levels on certain populations of Tcells (such as non-memory cells) but are present or expressed at relatively higher levels on certain other populations of T cells (such as memory cells). In one embodiment, the cells (such as the CD8⁺ cells or the T cells, e.g., CD3⁺ cells) are enriched for (i.e., positively selected for) cells that are positive or expressing high surface levels of CD45RO, CCR7, CD28, CD27, CD44,CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27,and/or IL7-Ra (CD 127). In some examples, CD8+ T cells are enriched for cells positive for CD45RO (or negative for CD45RA) and for CD62L. For example, CD3⁺, CD28⁺ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD 14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8⁺ T cells and CD4⁺ T cells further enhances efficacy.

In some embodiments, memory T cells are present in both CD62L⁺ and CD62L− subsets of CD8⁺ peripheral blood lymphocytes. PBMC can be enriched for or depleted of CD62L-CD8⁺ and/or CD62L⁺CD8⁺ fractions, such as using anti-CD8 and anti-CD62L antibodies. In some embodiments, a CD4⁺ T cell population and a CD8⁺ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

CD4+T helper cells are sorted into naive, central memory, and effector cells by identifying cell populations that have cell surface antigens. CD4+ lymphocytes can be obtained by standard methods. In some embodiments, naive CD4+T lymphocytes are CD45RO⁻,CD45RA⁺, CD62L⁺, CD4⁺ T cells. In some embodiments, central memory CD4⁺ cells are CD62L⁺ and CD45RO⁺. In some embodiments, effector CD4⁺ cells are CD62L− and CD45RO. In one example, to enrich for CD4⁺ cells by negative selection, a monoclonal antibody cocktail typically includes antibodies to CD14, CD20, CDl lb, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering/modification. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL. In certain embodiments, the modified cells are expanded without any stimulating agents. In certain embodiments, the modified cells are expanded in vivo.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16,HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### H. Pharmaceutical Compositions and Formulations

Also provided are populations of immune cells of the invention, compositions containing such cells and/or enriched for such cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1: Provision of a Single Cytokine in Cis (Autocrine Stimulation)

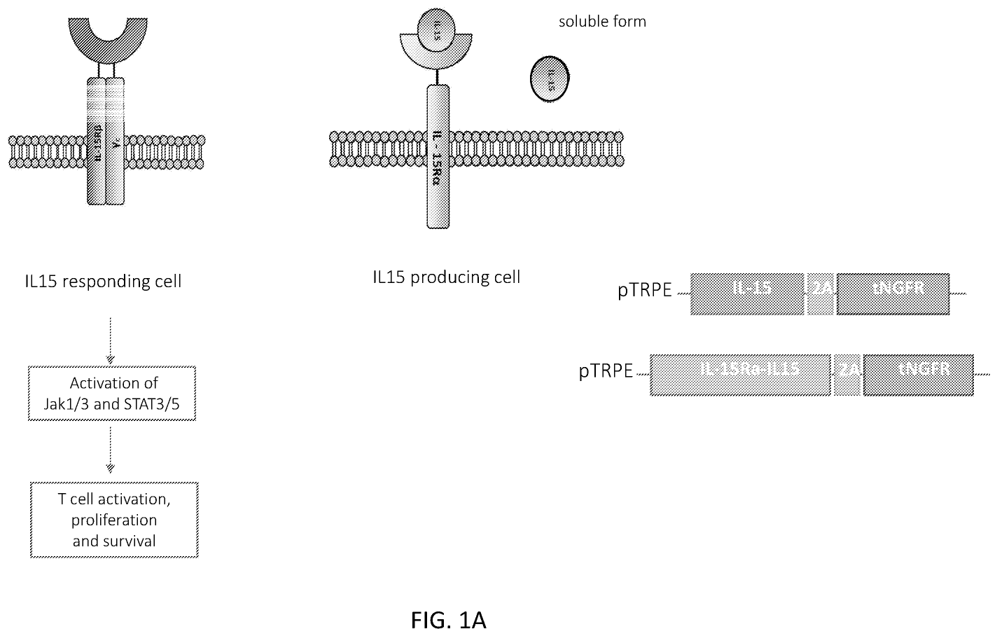

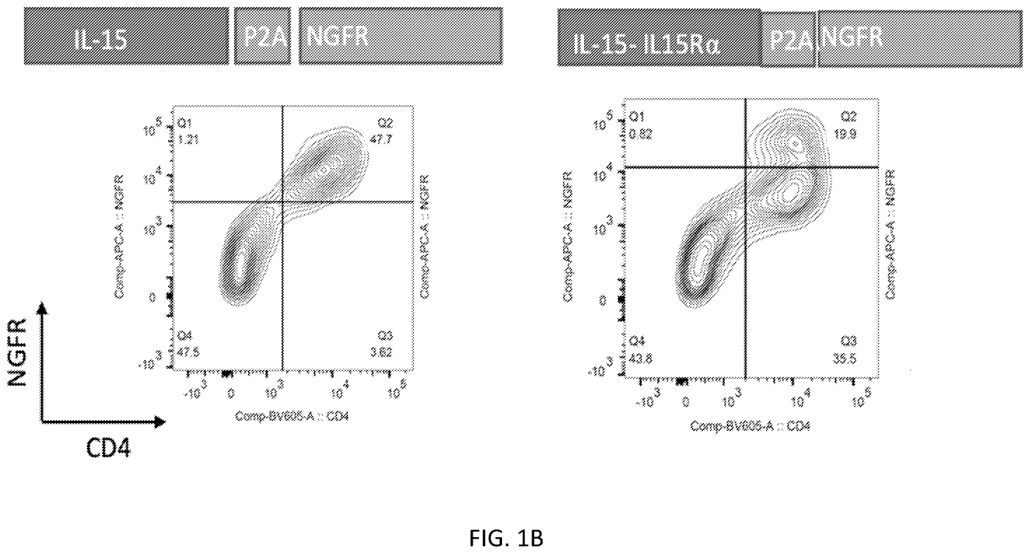

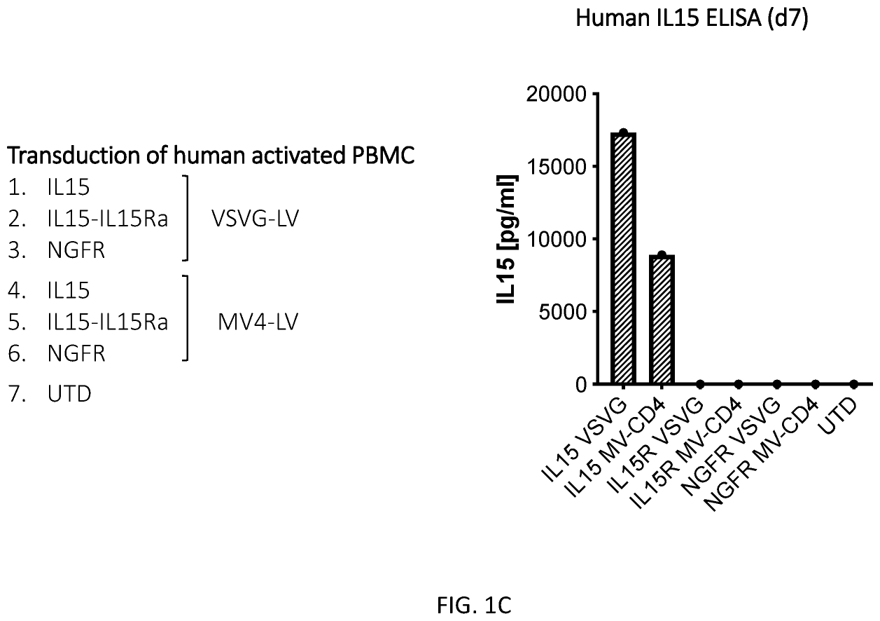

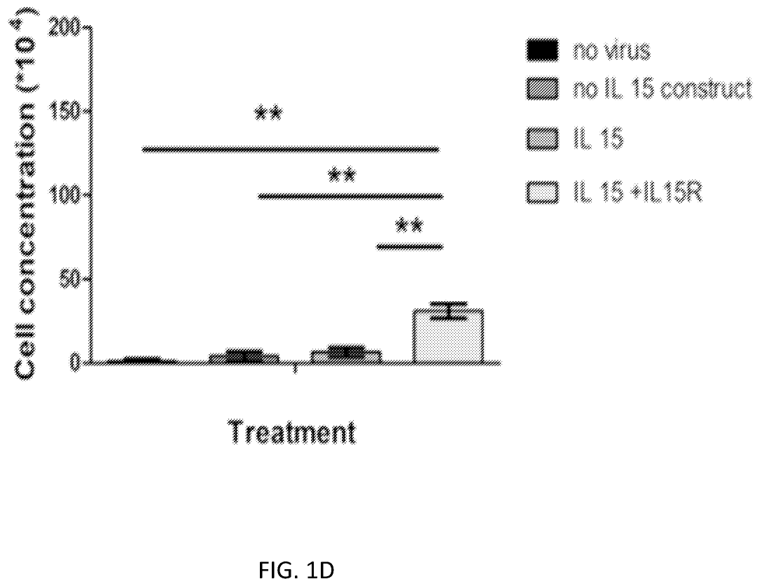

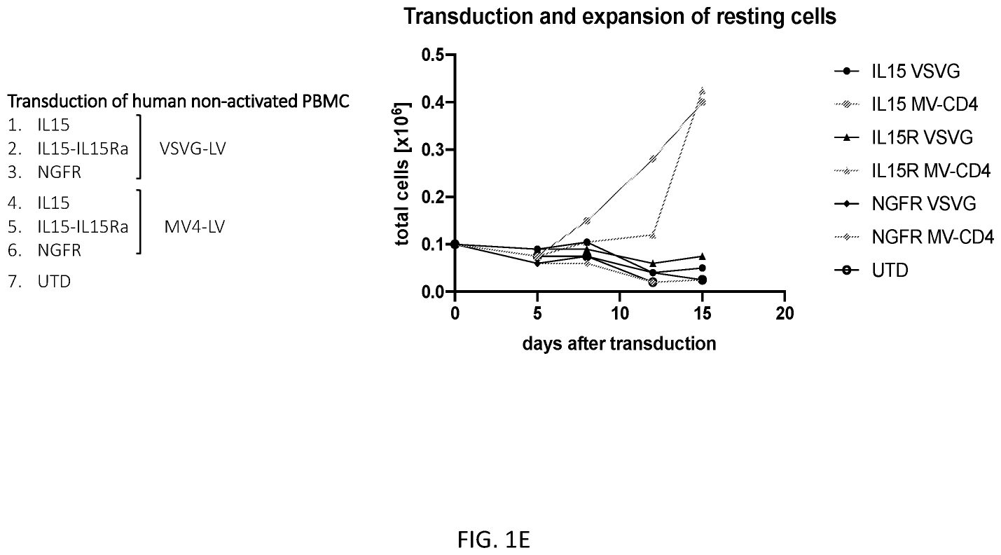

IL15 is a homeostatic cytokine that is required for the maintenance of CD8+ memory Tcells and NK cells, and promotes anti-tumor activity. IL15 signals through the IL15 receptor which is composed of a specific alpha chain (IL15Rα or IL15Ra) along with the shared IL2Rb and common gamma chain. Previous work has shown that a membrane bound version of IL15 (mbIL15) can be created by fusing IL15 directly to IL15Rα via a flexible linker that promotes IL15 signaling to the expressing cell (PMID: 27849617, US 2016/0158285 A1) (SEQ ID NO: 1,see Table 1). Herein, it was shown that incorporating mbIL15 in the lentiviral transfer genome increases CAR-T cell numbers 10-fold in vitro (FIGS. 1A-1D). This was particularly notable when non-activated (resting) T cells were transduced with the MV pseudotyped lentivector incorporating IL15 or IL15R/IL15 (FIG. 1E).

### Example 2: Delivery of a Single Cytokine, Controllable Signal by Transduction with Ectopic Cytokine Receptors

Hematopoietic cytokines such as erythropoietin, G-CSF, or thrombopoietin stimulate the production in the bone marrow of erythroid cells, granulocytic cells, and platelets, respectively. Cytokine-receptor interaction leads to downstream signaling (see e.g. FIG. 1 of Vainchenker & Constantinescu, Oncogene 2012; 32:2601-2013).

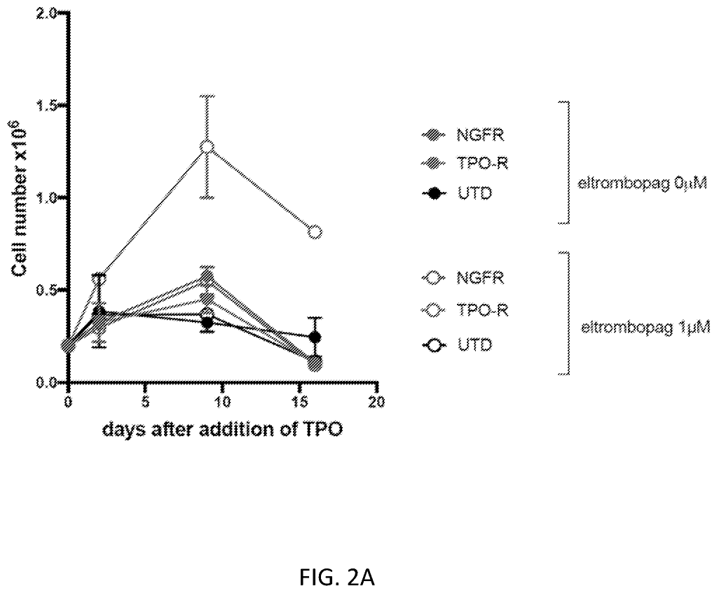

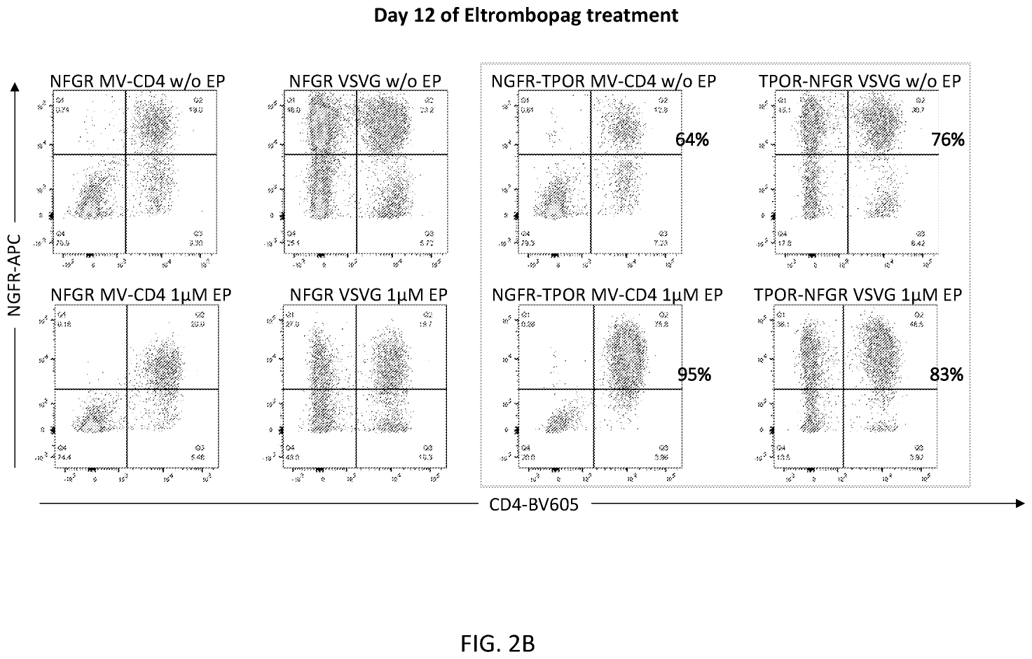

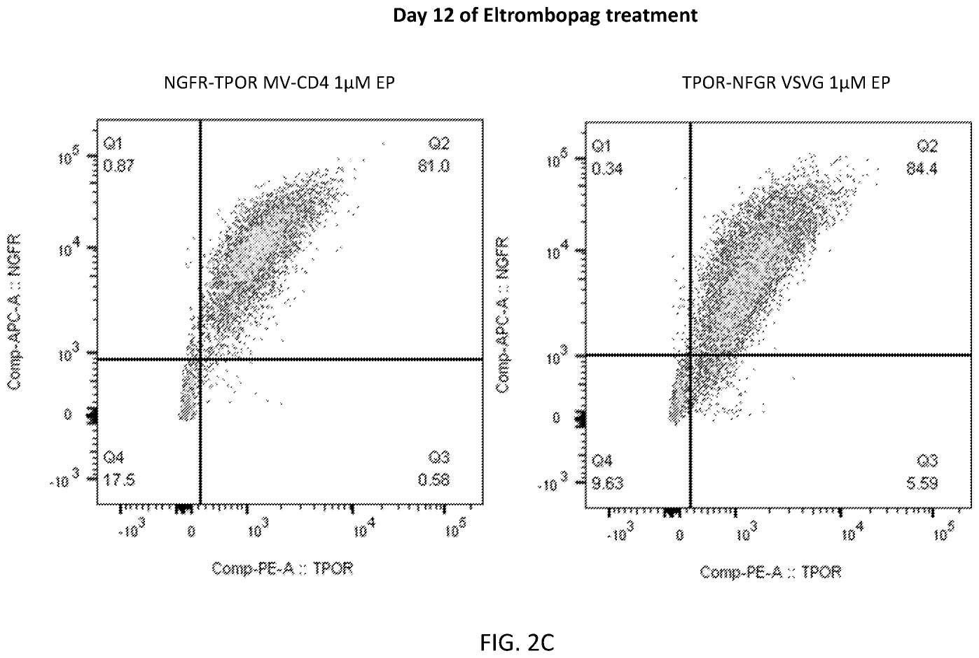

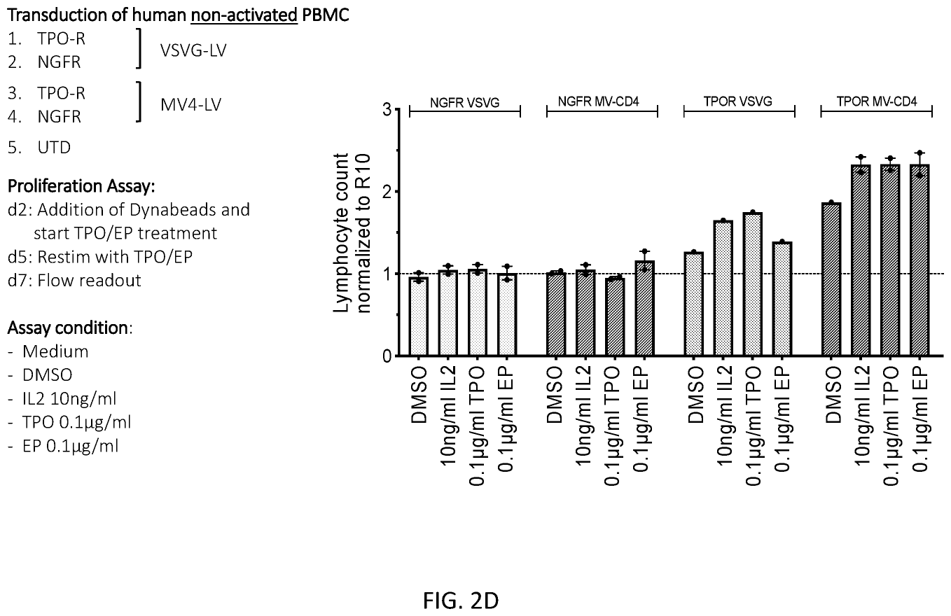

Recombinant erythropoietin (Epo) and G-CSF are commercially approved for the treatment for anemia of chronic kidney disease, and for chemotherapy-related neutropenia, respectively. Clinical development of a recombinant thrombopoietin (TPO) was superseded by two TPO receptor (TPO-R, also known as c-Mpl) peptidomimetic drugs that are now approved for the treatment of immune thrombocytopenic purpura (romiplostim, and eltrombopag). After transducing CART cells with Epo-R or TPO-R, other groups showed that delivery of erythropoietin, TPO or a TPO mimetic confers superior proliferation and anti-tumor functions (Nishimura et al., Blood. 2017; 130(25):2739-2749; Vinanica et al., Blood. 2020; 135(9):668-679). Results of the TPO-R experiments were recapitulated herein (FIGS. 2A-2D). T cells were transduced with the reporter NGFR, or with reporter+TPO-R and stimulated with eltrombopag, a specific small molecule agonist of TPOR, in vitro. T cells containing TPO-R showed superior proliferation (FIG. 2A). As a result, there was selective enrichment of the transduced cells, noted especially in T cells transduced with the MV pseudotyped lentivector (FIG. 2B). In FIG. 2C, it is shown that there is a 1:1 expression of the reporter NGFR with the TPO receptor after 12 days of eltrombopag treatment. Activation of the TPO receptor by its natural ligand thrombopoietin or by the agonist eltrombopag led to expansion of non-activated (resting) cells that were transduced using the MV pseudotyped lentivector but not resting cells that were transduced using the VSVG pseudotyped lentivector (FIG. 2D).

Stimulation of lymphocytes engineered to express the granulocyte colony-stimulating factor (G-CSF) receptor (G-CSFR) has not been attempted until now. rhG-CSF is widely used for the prevention of chemotherapy-associated neutropenia, generic equivalents to the original formulation NEUPOGEN® are now available, and there is at least one pegylated long-lasting rhG-CSF in widespread clinical use. Ectopic expression of the G-CSF receptor on T cells (e.g. CAR T cells) endows them with superior expansion kinetics upon stimulation with rhG-CSF in a controllable fashion. Use of an alternatively spliced class IV G-CSFR (gene name CSF3R) provides further JAK-STAT mediated stimulation compared with canonical (class I) CSF3R. This system is activatable by administration of G-CSF and subject to inhibition by administration of JAK2 or JAK1/2 inhibitors that are in routine clinical use (Mehta et al. (2014) Leukemia. 28(5):1041-51). Sequences of exemplary vectors comprising CSF3R, including the GCSFR isoform 1 (V1), isoform 4 (V4), and GCSFRd715, are listed below.

### Example 3: T Cells Transduced to Express (i) the Thrombopoietin Receptor (TPOR) or (ii) the GCF Receptor with a Deletion at Aa 715 (GCSFRd715)

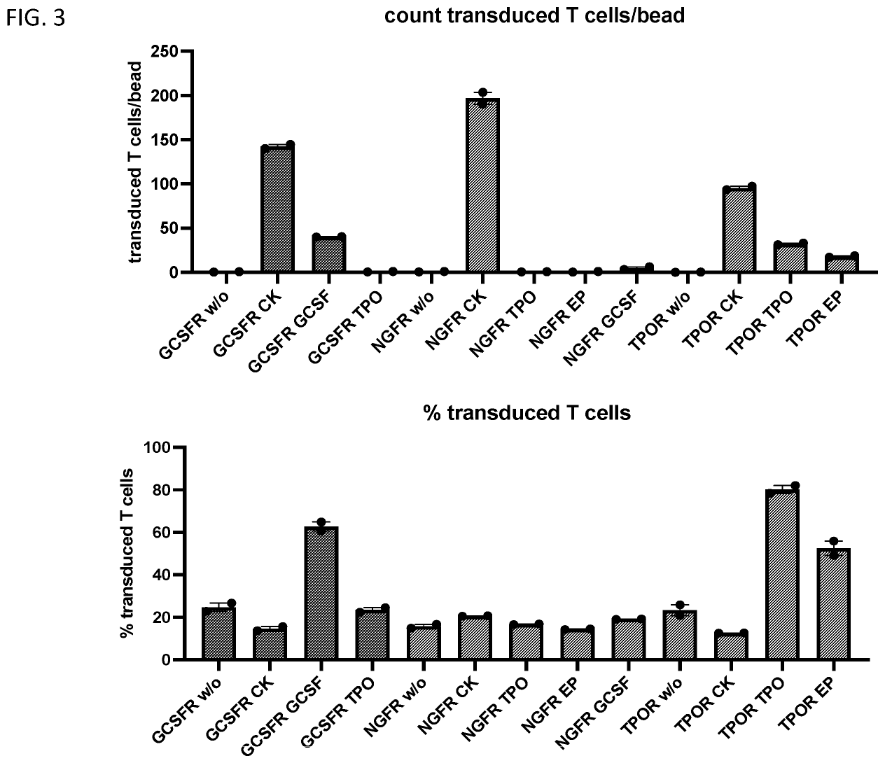

Healthy donor T cells were transduced to express (i) the thrombopoietin receptor (TPOR) or (ii) the GCF receptor with a deletion at aa 715 (GCSFRd715). T cells expressing GCSFRd715, TPOR, or the truncated NGFR as a control were stimulated for 7 days with IL-2/IL-7/IL-15 (all at 5 ng/ml; denoted as CK; positive control condition), 0.1 μg/ml recombinant thrombopoietin (TPO), 0.1 μg/ml TPO agonist eltrombopag (EP), or 0.1 μg/ml GCSF. Cell number (FIG. 3, top) or percent transgenic cells (FIG. 3, bottom) were quantified using flow cytometry at 7 days.

T cells showed robust stimulation in response to the CK positive control condition (FIG. 3, top). Negative control cells (NGFR) did not respond to GCSF, TPO or EP stimulation. GCSFRd715 cells responded to GCSF stimulation but not to TPO or EP. TPOR cells responded to TPO and EP but not to GCSF. Relative enrichment of transgenic cells was only shown when GCSFRd715 cells were stimulated with GCSF, or when TPOR cells were stimulated with TPO/RP (FIG. 3, bottom). These results demonstrated that the transgenic cytokine receptors are active, and can drive T cell proliferation via the non-canonical JAK2 pathway.

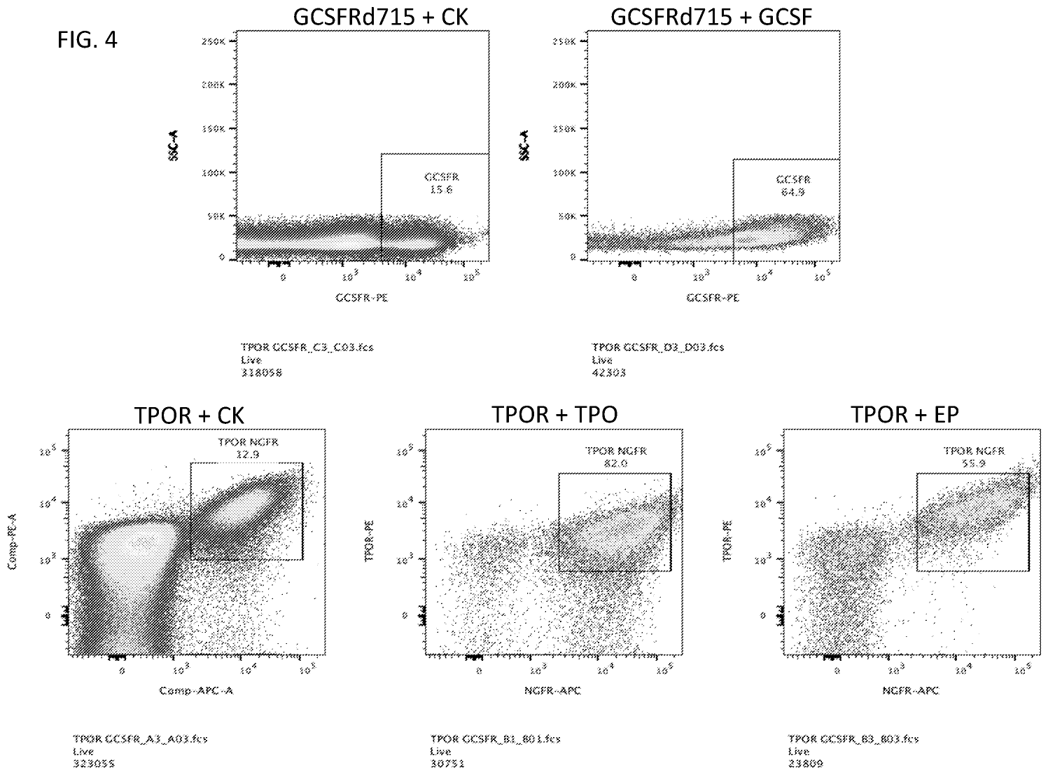

Exposure of transgenic cytokine receptor transduced cells to the cognate cytokine (i.e. GCSFRd715 to GCSF; TPOR to TPO or EP) led to enrichment of CAR expressing cells after 7 days (FIG. 4). Addition of GCSF enriched the GCSRFd715 transgenic cell fraction from 15.6% to 64.9% (FIG. 4, top panel). Addition of TPO enriched the TPOR fraction from 12.9% to 82% and addition of EP enriched the TPOR fraction from 12.9% to 55.9% (FIG. 4, bottom panel).

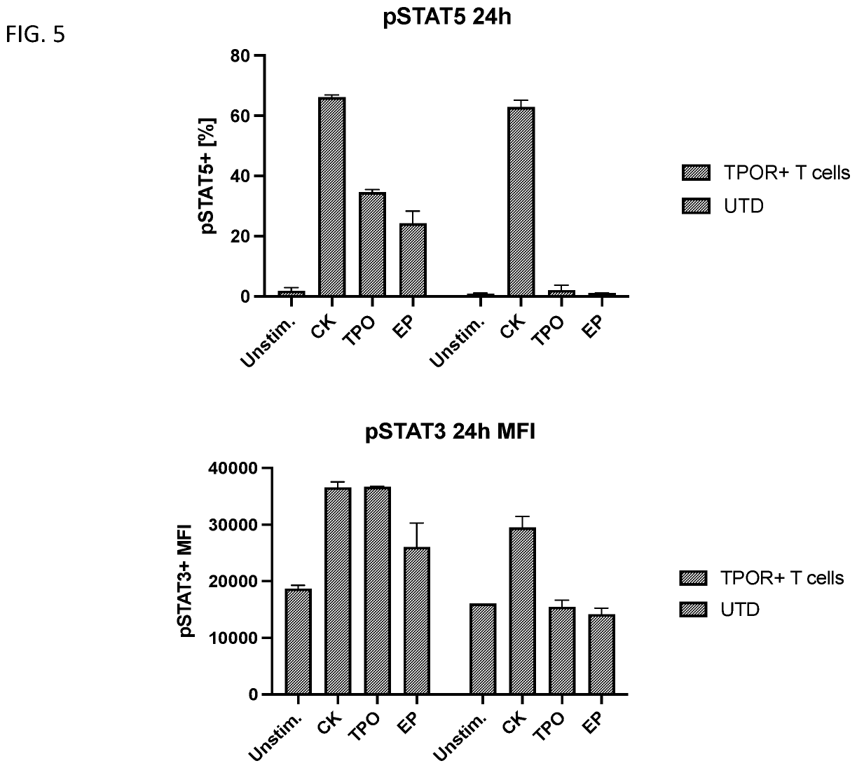

TPOR stimulation led to activation of STAT3 and STAT5 (FIG. 5). Healthy donor T cells expressing TPOR or untransduced control T cells were stimulated for 24 hours with 5 ng/ml IL-2/IL-7/IL-15 (CK), 0.1 μg/ml TPO, or 0.1 μg/ml Eltrombopag (EP). Following stimulation cells were fixed and the phosphorylation of STAT3 and STAT5 quantified using flow cytometry. These results showed that stimulation of TPOR expressing T cells with TPO or EP leads to STAT5 and STAT3 phosphorylation.

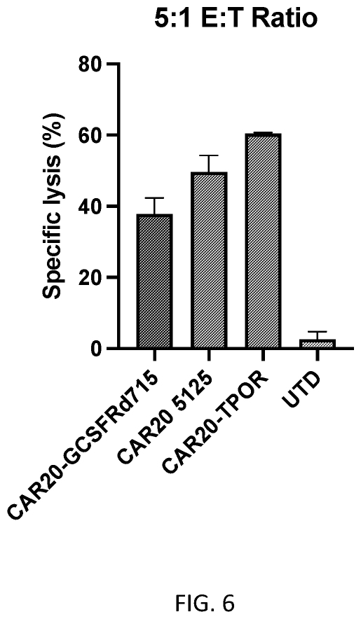

An anti-CD20 CAR (CAR20) was generated using an anti-CD20 scFv from the Leu16 clone (similar to Till et al, Blood 2012; 119:3940-3950). The scFv sequence was cloned into the pTRPE expression vector. Healthy donor CD8 T cells expressing CAR20, CAR20-TPOR, or CAR20-GCSFRd715 were incubated with CD20+ Raji tumor cells and target cell lysis was quantified by luminescence (FIG. 6). These results showed that expression of the transgenic cytokine receptors does not diminish the activity of CART-20 cells.

### Exemplary Sequences Used in the Invention:

```
mbIL15 (SEQ ID NO: 1):
MDWTWILFLVAAATRVHSNWVNVISDLKKIEDLIQSMHIDATLYTESDVHPSCKVTAMKCFLLELQV
ISLESGDASIHDTVENLIILANNSLSSNGNVTESGCKECEELEEKNIKEFLQSFVHIVQMFINTSSG
GGSGGGGSGGGGSGGGGSGGGSLQITCPPPMSVEHADIWVKSYSLYSRERYICNSGFKRKAGTSSLT
ECVLNKATNVAHWTTPSLKCIRDPALVHQRPAPPSTVTTAGVTPQPESLSPSGKEPAASSPSSNNTA
ATTAAIVPGSQLMPSKSPSTGTTEISSHESSHGTPSQTTAKNWELTASASHQPPGVYPQGHSDTTVA
ISTSTVLLCGLSAVSLLACYLKSRQTPPLASVEMEAMEALPVTWGTSSRDEDLENCSHHLSRMDYKD
DDDKDYKDDDDKDYKDDDDK
pTRPE CD20CAR-CSF3R d715 (SEQ ID NO: 2);
CD20CAR (SEQ ID NO: 13) comprises
nucleotides 3390-5489 (underlined);
CSF3R d715 (SEQ ID NO: 11) comprises nucleotides
5562-7709 (double underlined):
gggtctctctggttagaccagatctgagcctgggagctctctggctaactagggaacccactgcttaag
cctcaataaagcttgccttgagtgcttcaagtagtgtgtgcccgtctgttgtgtgactctggtaactag
agatccctcagacccttttagtcagtgtggaaaatctctagcagtggcgcccgaacagggacttgaaag
cgaaagggaaaccagaggagctctctcgacgcaggactcggcttgctgaagcgcgcacggcaagaggcg
aggggcggcgactggtgagtacgccaaaaattttgactagcggaggctagaaggagagagatgggtgcg
agcgggggagaattagatcgcgatgggaaaaaattcggttaaggccagggggaaagaaaaaatataaat
taaaacatatagtatgggcaagcagggagctagaacgattcgcagttaatcctggcctgttagaaacat
cagaaggctgtagacaaatactgggacagctacaaccatcccttcagacaggatcagaagaacttagat
cattatataatacagtagcaaccctctattgtgtgcatcaaaggatagagataaaagacaccaaggaag
ctttagacaagatagaggaagagcaaaaaaaagtaagaccaccgcacagcaagcggccgctgatcttca
gacctggaggaggagatatgagggacaattggagaagtgaattatataaatataaagtagtaaaaattg
aaccattaggagtagcacccaccaaggcaaagagaaagtggtgcagagagaaaaaagagcagtggggaa
taggagctttgttccttgggttcttgggagcagcaggaagcactatgggcgcagcgtcaatgacgctga
cggtacaggccagacaattattgtctggtatagtgcagcagcagaacaatttgctgagggctattgagg
cgcaacagcatctgttgcaactcacagtctggggcatcaagcagctccaggcaagaatcctggctgtgg
aaagatacctaaaggatcaacagctcctggggatttggggttgctctggaaaactcatttgcaccactg
ctgtgccttggaatgctagttggagtaataaatctctggaacagatttggaatcacacgacctggatgg
agtgggacagagaaattaacaattacacaagcttaatacactccttaattgaagaatcgcaaaaccagc
aagaaaagaatgaacaagaattattggaattagataaatgggcaagtttgtggaattggtttaacataa
caaattggctgtggtatataaaattattcataatgatagtaggaggcttggtaggtttaagaatagttt
ttgctgtactttctatagtgaatagagttaggcagggatattcaccattatcgtttcagacccacctcc
caaccccgaggggacccgacaggcccgaaggaatagaagaagaaggtggagagagagacagagacagat
ccattcgattagtgaacggatctcgacggtatcgattagactgtagcccaggaatatggcagctagatt
gtacacatttagaaggaaaagttatcttggtagcagttcatgtagccagtggatatatagaagcagaag
taattccagcagagacagggcaagaaacagcatacttcctcttaaaattagcaggaagatggccagtaa
aaacagtacatacagacaatggcagcaatttcaccagtactacagttaaggccgcctgttggtgggcgg
ggatcaagcaggaatttggcattccctacaatccccaaagtcaaggagtaatagaatctatgaataaag
aattaaagaaaattataggacaggtaagagatcaggctgaacatcttaagacagcagtacaaatggcag
tattcatccacaattttaaaagaaaaggggggattggggggtacagtgcaggggaaagaatagtagaca
taatagcaacagacatacaaactaaagaattacaaaaacaaattacaaaaattcaaaattttcgggttt
attacagggacagcagagatccagtttggctgcatacgcgtcgtgaggctccggtgcccgtcagtgggc
agagcgcacatcgcccacagtccccgagaagttggggggaggggtcggcaattgaaccggtgcctagag
aaggtggcgcggggtaaactgggaaagtgatgtcgtgtactggctccgcctttttcccgagggtggggg
agaaccgtatataagtgcagtagtcgccgtgaacgttctttttcgcaacgggtttgccgccagaacaca
ggtaagtgccgtgtgtggttcccgcgggcctggcctctttacgggttatggcccttgcgtgccttgaat
tacttccacctggctgcagtacgtgattcttgatcccgagcttcggttggaagtgggtgggagaggttc
gaggccttgcgcttaaggagccccttcgcctcgtgcttgagttgaggcctggcctgggcgctggggccg
ccgcgtgcgaatctggtggcaccttcgcgcctgtctcgctgctttcgataagtctctagccatttaaaa
tttttgatgacctgctgcgacgctttttttctggcaagatagtcttgtaaatgcgggccaagatctgca
cactggtatttcggtttttggggccgcgggcggcgacggggcccgtgcgtcccagcgcacatgttcggc
gaggcggggcctgcgagcgcggccaccgagaatcggacgggggtagtctcaagctggccggcctgctct
ggtgcctggcctcgcgccgccgtgtatcgccccgccctgggcggcaaggctggcccggtcggcaccagt
tgcgtgagcggaaagatggccgcttcccggccctgctgcagggagctcaaaatggaggacgcggcgctc
gggagagcggggggtgagtcacccacacaaaggaaaagggcctttccgtcctcagccgtcgcttcatgt
gactccactgagtaccgggcgccgtccaggcacctcgattagttctcgtgcttttggagtacgtcgtct
ttaggttggggggaggggttttatgcgatggagtttccccacactgagtgggtggagactgaagttagg
ccagcttggcacttgatgtaattctccttggaatttgccctttttgagtttggatcttggttcattctca
agcctcagacagtggttcaaagtttttttcttccatttcaggtgtcgtgagctagctctagagccgccat
ggagacagacacactcctgctatgggtgctgctgctctgggttccaggttccacaggt
gacattgtgctgacccaatctccagctatcctgtctgcatctccaggggagaaggtcacaatgacttgcagggc
cagctcaagtgtaaattacatggactggtaccagaagaagccaggatcctcccccaaaccctggatttatgccaca
accatcctggcttctggagtccctgctcgcttcagtggcagtgggtctgggacctcttactctctcacaatcagca
gaggctgaagatgctgccacttattactgccagcagtggagttttaatccacccacgttcggaggggggaccagt
ggaagctggaaataaaaggcagtactagcggtggtggctccgggggcggttccggtgggggcggcagca
gcgaggtgcagctgcagcagtctggggctgagctggtgaagcctggggcctcagtgaagatgtcctgc
aaggcttctggctacacatttaccagttacaatatgcactgggtaaagcagacacctggacagggcct
ggaatggattggagctatttatccaggaaatggtgatacttcctacaatcagaagttcaaaggcaag
gccacattgactgcagacaaatcctccagcacagcctacatgcagctcagcagcctgacatctgagg
actctgcggactattactgtgcaagatctaattattacggtagtagctactggttcttcgatgtctgg
ggcgcagggaccacggtcaccgtctcctcactcgacgaatctaagtacggaccgccctgccccccttg
ccctgcccccgagttcctgggcggacccagcgtgttcctgttcccccccaagcccaaggacaccctga
tgatcagccggacccccgaggtgacctgcgtggtggtggacgtgagccaggaagatcccgaggtcca
gttcaattggtacgtggacggcgtggaagtgcacaacgccaagaccaagcccagagaggaacagtt
caacagcacctaccgggtggtgtctgtgctgaccgtgctgcaccaggactggctgaacggcaaagaa
tacaagtgcaaggtgtccaacaagggcctgcccagcagcatcgaaaagaccatcagcaaggccaagggc
cagcctcgcgagccccaggtgtacaccctgcctccctcccaggaagagatgaccaagaaccaggtgtc
cctgacctgcctggtgaagggcttctaccccagcgacatcgccgtggagtgggagagcaacggccag
cctgagaacaactacaagaccacccctcccgtgctggacagcgacggcagcttcttcctgtacagcc
ggctgaccgtggacaagagccggtggcaggaaggcaacgtctttagctgcagcgtgatgcacgaggcc
ctgcacaaccactacacccagaagagcctgagcctgtccctgggcaagatgttctgggtgctggtggt
ggtgggggggtgctggcctgctacagcctgctggtgacagtggccttcatcatcttttgggtgcgga
gcaagcggagcagaggcggccacagcgactacatgaacatgacccccagacggcctggccccacccg
gaagcactaccagccctacgccccacccagggactttgccgcctacagaagcaaacggggcagaaaga
aactcctgtatatattcaaacaaccatttatgagaccagtacaaactactcaagaggaagatggctgt
agctgccgatttccagaagaagaagaaggaggatgtgaactgcgggtgaagttcagcagaagcgccg
acgcccctgcctaccagcagggccagaatcagctgtacaacgagctgaacctgggcagaagggaaga
gtacgacgtcctggataagcggagaggccgggaccctgagatgggggcaagcctcggcggaagaaccc
ccaggaaggcctgtataacgaactgcagaaagacaagatggccgaggcctacagcgagatcggcatgaa
gggcgagcggaggcggggcaagggccacgacggcctgtatcagggcctgtccaccgccaccaagga
tacctacgacgccctgcacatgcaggccctgcccccaaggctcgagggcggcggagagggcagagg
aagtcttctaacatgcggtgacgtggaggagaatcccggccctaggATGGCAAGGCTGGGAAACTGCAG
CCTGACTTGGGCTGCCCTGATCATCCTGCTGCTCCCCGGAAGTCT
GGAGGAGTGCGGGCACATCAGTGTCTCAGCCCCCATCGTCCACCT
GGGGGATCCCATCACAGCCTCCTGCATCATCAAGCAGAACTGCAG
CCATCTGGACCCGGAGCCACAGATTCTGTGGAGACTGGGAGCAGA
GCTTCAGCCCGGGGGCAGGCAGCAGCGTCTGTCTGATGGGACCCA
GGAATCTATCATCACCCTGCCCCACCTCAACCACACTCAGGCCTT
TCTCTCCTGCTGCCTGAACTGGGGCAACAGCCTGCAGATCCTGGA
CCAGGTTGAGCTGCGCGCAGGCTACCCTCCAGCCATACCCCACAA
CCTCTCCTGCCTCATGAACCTCACAACCAGCAGCCTCATCTGCCA
GTGGGAGCCAGGACCTGAGACCCACCTACCCACCAGCTTCACTCT
GAAGAGTTTCAAGAGCCGGGGCAACTGTCAGACCCAAGGGGACTC
CATCCTGGACTGCGTGCCCAAGGACGGGCAGAGCCACTGCTGCAT
CCCACGCAAACACCTGCTGTTGTACCAGAATATGGGCATCTGGGT
GCAGGCAGAGAATGCGCTGGGGACCAGCATGTCCCCACAACTGTG
TCTTGATCCCATGGATGTTGTGAAACTGGAGCCCCCCATGCTGCG
GACCATGGACCCCAGCCCTGAAGCGGCCCCTCCCCAGGCAGGCTG
CCTACAGCTGTGCTGGGAGCCATGGCAGCCAGGCCTGCACATAAA
TCAGAAGTGTGAGCTGCGCCACAAGCCGCAGCGTGGAGAAGCCAG
CTGGGCACTGGTGGGCCCCCTCCCCTTGGAGGCCCTTCAGTATGA
GCTCTGCGGGCTCCTCCCAGCCACGGCCTACACCCTGCAGATACG
CTGCATCCGCTGGCCCCTGCCTGGCCACTGGAGCGACTGGAGCCC
CAGCCTGGAGCTGAGAACTACCGAACGGGCCCCCACTGTCAGACT
GGACACATGGTGGCGGCAGAGGCAGCTGGACCCCAGGACAGTGCA
GCTGTTCTGGAAGCCAGTGCCCCTGGAGGAAGACAGCGGACGGAT
CCAAGGTTATGTGGTTTCTTGGAGACCCTCAGGCCAGGCTGGGGC
CATCCTGCCCCTCTGCAACACCACAGAGCTCAGCTGCACCTTCCA
CCTGCCTTCAGAAGCCCAGGAGGTGGCCCTTGTGGCCTATAACTC
AGCCGGGACCTCTCGTCCCACTCCGGTGGTCTTCTCAGAAAGCAG
AGGCCCAGCTCTGACCAGACTCCATGCCATGGCCCGAGACCCTCA
CAGCCTCTGGGTAGGCTGGGAGCCCCCCAATCCATGGCCTCAGGG
CTATGTGATTGAGTGGGGCCTGGGCCCCCCCAGCGCGAGCAATAG
CAACAAGACCTGGAGGATGGAACAGAATGGGAGAGCCACGGGGTT
TCTGCTGAAGGAGAACATCAGGCCCTTTCAGCTCTATGAGATCAT
CGTGACTCCCTTGTACCAGGACACCATGGGACCCTCCCAGCATGT
CTATGCCTACTCTCAAGAAATGGCTCCCTCCCATGCCCCAGAGCT
GCATCTAAAGCACATTGGCAAGACCTGGGCACAGCTGGAGTGGGT
GCCTGAGCCCCCTGAGCTGGGGAAGAGCCCCCTTACCCACTACAC
CATCTTCTGGACCAACGCTCAGAACCAGTCCTTCTCCGCCATCCT
GAATGCCTCCTCCCGTGGCTTTGTCCTCCATGGCCTGGAGCCCGC
CAGTCTGTATCACATCCACCTCATGGCTGCCAGCCAGGCTGGGGC
CACCAACAGTACAGTCCTCACCCTGATGACCTTGACCCCAGAGGG
GTCGGAGCTACACATCATCCTGGGCCTGTTCGGCCTCCTGCTGTT
GCTCACCTGCCTCTGTGGAACTGCCTGGCTCTGTTGCAGCCCCAA
CAGGAAGAATCCCCTCTGGCCAAGTGTCCCAGACCCAGCTCACAG
CAGCCTGGGCTCCTGGGTGCCCACAATCATGGAGGAGGATGCCTT
CCAGCTGCCCGGCCTTGGCACGCCACCCATCACCAAGCTCACAGT
GCTGGAGGAGGATGAAAAGAAGCCGGTGCCCTGGGAGTCCCATAA
CAGCTCATAGagcggccgcgtcgacaatcaacctctggattacaaaatttgtgaaagattgactggta
ttcttaactatgttgctccttttacgctatgtggatacgctgctttaatgcctttgtatcatgctattg
cttcccgtatggctttcattttctcctccttgtataaatcctggttgctgtctctttatgaggagttgtg
gcccgttgtcaggcaacgtggcgtggtgtgcactgtgtttgctgacgcaacccccactggttggggcatt
gccaccacctgtcagctcctttccgggactttcgctttccccctccctattgccacggcggaactcatcg
ccgcctgccttgcccgctgctggacaggggctcggctgttgggcactgacaattccgtggtgttgtcgggg
aagctgacgtcctttccttggctgctcgcctgtgttgccacctggattctgcgcgggacgtcctctgcttac
gtcccttcggccctcaatccagcggaccttccttcccgcggcctgctgccggctctgcggcctcttccgcgt
cttcgccttcgccctcagacgagtcggatctccctttgggccgcctccccgcctggaattcgagctcg
gtacctttaagaccaatgacttacaaggcagctgtagatcttagccactttttaaaagaaaaggggggact
ggaagggctaattcactcccaacgaagacaagatctgctttttgcttgtactgggtctctctggttaga
ccagatctgagcctgggagctctctggctaactagggaacccactgcttaagcctcaataaagcttgcct
tgagtgcttcaagtagtgtgtgcccgtctgttgtgtgactctggtaactagagatccctcagaccctttt
agtcagtgtggaaaatctctagcagtagtagttcatgtcatcttattattcagtatttataacttgcaaa
gaaatgaatatcagagagtgagaggaacttgtttattgcagcttataatggttacaaataaagcaatagc
atcacaaatttcacaaataaagcatttttttcactgcattctagttgtggtttgtccaaactcatcaatg
tatcttatcatgtctggctctagctatcccgcccctaactccgcccagttccgcccattctccgccccat
ggctgactaattttttttatttatgcagaggccgaggccgcctcggcctctgagctattccagaagtagt
gaggaggcttttttggaggcctagctagggacgtacccaattcgccctatagtgagtcgtattacgcgcgc
tcactggccgtcgttttacaacgtcgtgactgggaaaaccctggcgttacccaacttaatcgccttgcagc
acatccccctttcgccagctggcgtaatagcgaagaggcccgcaccgatcgcccttcccaacagttgcgca
gcctgaatggcgaatgggacgcgccctgtagcggcgcattaagcgcggcgggtgtggtggttacgcgcagcg
tgaccgctacacttgccagcgccctagcgcccgctcctttcgctttcttcccttcctttctcgccacgttcgc
gccggctttccccgtcaagctctaaatcgggggctccctttagggttccgatttagtgctttacggcacctc
gaccccaaaaaacttgattagggtgatggttcacgtagtgggccatcgccctgatagacggtttttcgccctt
tgacgttggagtccacgttctttaatagtggactcttgttccaaactggaacaacactcaaccctatctcggt
ctattcttttgatttataagggattttgccgatttcggcctattggttaaaaaatgagctgatttaacaaaaat
ttaacgcgaattttaacaaaatattaacgcttacaatttaggtggcacttttcggggaaatgtgcgcggaaccc
ctatttgtttatttttctaaatacattcaaatatgtatccgctcatgagacaataaccctgataaatgcttcaa
taatattgaaaaaggaagagtatgagtattcaacatttccgtgtcgcccttattcccttttttgcggcattttg
ccttcctgtttttgctcacccagaaacgctggtgaaagtaaaagatgctgaagatcagttgggtgcacgagtgg
gttacatcgaactggatctcaacagcggtaagatccttgagagttttcgccccgaagaacgttttccaatgatg
agcacttttaaagttctgctatgtggcgcggtattatcccgtattgacgccgggcaagagcaactcggtcgccg
catacactattctcagaatgacttggttgagtactcaccagtcacagaaaagcatcttacggatggcatgacag
taagagaattatgcagtgctgccataaccatgagtgataacactgcggccaacttacttctgacaacgatcgga
ggaccgaaggagctaaccgcttttttgcacaacatgggggatcatgtaactcgccttgatcgttgggaaccgga
gctgaatgaagccataccaaacgacgagcgtgacaccacgatgcctgtagcaatggcaacaacgttgcgcaaac
tattaactggcgaactacttactctagcttcccggcaacaattaatagactggatggaggcggataaagttgca
ggaccacttctgcgctcggcccttccggctggctggtttattgctgataaatctggagccggtgagcgtgggtc
ttcgcggtacattgcagcactggggccagatggtaagccctcccgtatcgtagttatctacacgacggggagtc
aggcaactatggatgaacgaaatagacagatcgctgagataggtgcctcactgattaagcattggtaactgtca
gaccaagtttactcatatatactttagattgatttaaaacttcatttttaatttaaaaggatctaggtgaagat
cctttttgataatctcatgaccaaaatcccttaacgtgagttttcgttccactgagcgtcagaccccgtagaaa
agatcaaaggatcttcttgagatcctttttttctgcgcgtaatctgctgcttgcaaacaaaaaaaccaccgct
accagcggtggtttgtttgccggatcaagactaccaactctttttccgaaggtaactggcttcgagcagag
cgcagataccaaatactgttcttctagtgtagccgtagttaggccaccacttcaagaactctgtagcaccg
cctacatacctcgctctgctaatcctgttaccagtggctgctgccagtggcgataagtcgtgtcttaccgg
gttggactcaagacgatagttaccggataaggcgcagcggtcgggctgaacggggggttcgtgcacacagcc
cagcttggagcgaacgacctacaccgaactgagatacctacagcgtgagctatgagaaagcgccacgcttccc
gaagggagaaaggcggacaggtatccggtaagcggcagggtcggaacaggagagcgcacgagggagcttccagg
gggaaacgcctggtatctttatagtcctgtcgggtttcgccacctctgacttgagcgtcgatttttgtgatgc
tcgtcaggggggcggagcctatggaaaaacgccagcaacgcggcctttttacggttcctggccttttgctggc
cttttgctcacatgttctttcctgcgttatcccctgattctgtggataaccgtattaccgcctttgagtgagc
tgataccgctcgccgcagccgaacgaccgagcgcagcgagtcagtgagcgaggaagcggaagagcgcccaata
cgcaaaccgcctctccccgcgcgttggccgattcattaatgcagctggcacgacaggtttcccgactggaaag
cgggcagtgagcgcaacgcaattaatgtgagttagctcactcattaggcaccccaggctttacactttatgct
tccggctcgtatgttgtgtggaattgtgagcggataacaatttcacacaggaaacagctatgaccatgattac
gccaagcgcgcaattaaccctcactaaagggaacaaaagctggagctgcaagcttaatgtagtcttatgcaata
ctcttgtagtcttgcaacatggtaacgatgagttagcaacatgccttacaaggagagaaaaagcaccgtgcatg
ccgattggtggaagtaaggtggtacgatcgtgccttattaggaaggcaacagacgggtctgacatggattgga
cgaaccactgaattgccgcattgcagagatattgtatttaagtgcctagctcgatacataaac
pTRPE CD20CAR-CSF3Rv1 (SEQ ID NO: 3);
CSF3Rv1 (SEQ ID NO: 12) comprises
nucleotides 5562-8072 (double underlined):
gggtctctctggttagaccagatctgagcctgggagctctctggctaactagggaacccactgcttaagcctcaa
taaagcttgccttgagtgcttcaagtagtgtgtgcccgtctgttgtgtgactctggtaactagagatccctcaga
cccttttagtcagtgtggaaaatctctagcagtggcgcccgaacagggacttgaaagcgaaagggaaaccagagg
agctctctcgacgcaggactcggcttgctgaagcgcgcacggcagaggcgaggggcggcgactggtgagtacgcc
aaaaaattttgactagcggaggctagaaggagagagatgggtgcgagagcgtcagtattaagcgggggagaatta
gatcgcgatgggaaaaaattcggttaaggccagggggaaagaaaaaatataaattaaaacatatagtatgggcaa
gcagggagctagaacgattcgcagttaatcctggcctgttagaaacatcagaaggctgtagacaaatactgggac
agctacaaccatcccttcagacaggatcagaagaacttagatcattatataatacagtagcaaccctctattgtg
tgcatcaaaggatagagataaaagacaccaaggaagctttagacaagatagaggaagagcaaaacaaaagtaaga
ccaccgcacagcaagcggccgctgatcttcagacctggaggaggagatatgagggacaattggagaagtgaatta
tataaatataaagtagtaaaaattgaaccattaggagtagcacccaccaaggcaaagagaagagtggtgcagaga
gaaaaaagagcagtgggaataggagctttgttccttgggttcttgggagcagcaggaagcactatgggcgcagcg
tcaatgacgctgacggtacaggccagacaattattgtctggtatagtgcagcagcagaacaatttgctgagggct
attgaggcgcaacagcatctgttgcaactcacagtctggggcatcaagcagctccaggcaagaatcctggctgtg
gaaagatacctaaaggatcaacagctcctggggatttggggttgctctggaaaactcatttgcaccactgctgtg
ccttggaatgctagttggagtaataaatctctggaacagatttggaatcacacgacctggatggagtgggacaga
gaaattaacaattacacaagcttaatacactccttaattgaagaatcgcaaaaccagcaagaaaagaatgaacaa
gaattattggaattagataaatgggcaagtttgtggaattggtttaacataacaaattggctgtggtatataaaa
ttattcataatgatagtaggaggcttggtaggtttaagaatagtttttgctgtactttctatagtgaatagagtt
aggcagggatattcaccattatcgtttcagacccacctcccaaccccgaggggacccgacaggcccgaaggaata
gaagaagaaggtggagagagagacagagacagatccattcgattagtgaacggatctcgacggtatcgattagac
tgtagcccaggaatatggcagctagattgtacacatttagaaggaaaagttatcttggtagcagttcatgtagcc
agtggatatatagaagcagaagtaattccagcagagacagggcaagaaacagcatacttcctcttaaaattagca
ggaagatggccagtaaaaacagtacatacagacaatggcagcaatttcaccagtactacagttaaggccgcctgt
tggtgggcggggatcaagcaggaatttggcattccctacaatccccaaagtcaaggagtaatagaatctatgaat
aaagaattaaagaaaattataggacaggtaagagatcaggctgaacatcttaagacagcagtacaaatggcagta
ttcatccacaattttaaaagaaaaggggggattggggggtacagtgcaggggaaagaatagtagacataatagca
acagacatacaaactaaagaattacaaaaacaaattacaaaaattcaaaattttcgggtttattacagggacagca
gagatccagtttggctgcatacgcgtcgtgaggctccggtgcccgtcagtgggcagagccacatcgcccacagtcc
gccgagaagttggggggaggggtcggcaattgaaccggtgcctagagaaggtggcgcggggtaaactgggaaagtg
atgtcgtgtactggctccgcctttttcccgagggtgggggagaaccgtatataagtgcagtagtcgccgtgaacgt
tctttttcgcaacgggtttgccgccagaacacaggtaagtgccgtgtgtggttcccgcgggcctggcctctttacg
ggttatggcccttgcgtgccttgaattacttccacctggctgcagtacgtgattcttgatcccgagcttcgggttg
gaagtgggtgggagagttcgaggccttgcgcttaaggagccccttcgcctcgtgcttgagttgaggcctggcctgg
gcgctggggccgccgcgtgcgaatctggtggcaccttcgcgcctgtctcgctgctttcgataagtctctagccatt
taaaatttttgatgacctgctgcgacgctttttttctggcaagatagtcttgtaaatgcgggccaagatctgcaca
ctggtatttcggtttttggggccgcgggggcgacggggcccgtgcgtcccagcgcacatgttcggcgaggggggcc
tgcgagcgcggccaccgagaatcggacgggggtagtctcaagctggccggcctgctctggtgcctggcctcgcgcc
gccgtgtatcgccccgccctgggcggcaaggctggcccggtcggcaccagttgcgtgagcggaaagatggccgctt
tcccggccctgctgcagggagctcaaaatggaggacgcggcgctcgggagagcggggggtgagtcacccacacaaa
ggaaaagggcctttccgtcctcagccgtcgcttcatgtgactccactgagtaccgggcgccgtccaggcacctcga
ttagttctcgtgcttttggagtacgtcgtctttaggttggggggaggggttttatgcgatggagtttccccacact
gagtgggtggagactgaagttaggccagcttggcacttgatgtaattctccttggaatttgccctttttgagtttg
gatcttggttcattctcaagcctcagacagtggttcaaagtttttttcttccatttcaggtgtcgtgagctagctc
tagagccgccatggagacagacacactcctgctatgggtgctgctgctctgggttccaggttccacaggtgacatt
gtgctgacccaatctccagctatcctgtctgcatctccaggggagaaggtcacaatgacttgcagggccagctcaa
gtgtaaattacatggactggtaccagaagaagccaggatcctcccccaaaccctggatttatgccacatccaacct
ggcttctggagtccctgctcgcttcagtggcagtgggtctgggacctcttactctctcacaatcagcagagtggag
gctgaagatgctgccacttattactgccagcagtggagttttaatccacccacgttcggaggggggaccaagctgg
aaataaaaggcagtactagcggtggtggctccgggggcggttccggtgggggcggcagcagcgaggtgcagctgca
gcagtctggggctgagctggtgaagcctggggcctcagtgaagatgtcctgcaaggcttctggctacacatttacc
agttacaatatgcactgggtaaagcagacacctggacagggcctggaatggattggagctatttatccaggaaatg
gtgatacttcctacaatcagaagttcaaaggcaaggccacattgactgcagacaaatcctccagcacagcctacat
gcagctcagcagcctgacatctgaggactctgcggactattactgtgcaagatctaattattacggtagtagctac
tggttcttcgatgtctggggcgcagggaccacggtcaccgtctcctcactcgacgaatctaagtacggaccgccct
tgccccccttgccctgcccccgagttcctgggcggacccagcgtgttcctgttcccccccaagcccaaggacaccc
tgatgatcagccggacccccgaggtgacctgcgtggtggtggacgtgagccaggaagatcccgaggtccagttcaa
ttggtacgtggacggcgtggaagtgcacaacgccaagaccaagcccagagaggaacagttcacagcacctaccgga
agtggtgtctgtgctgaccgtgctgcaccaggactggctgaacggcaaagaatacaagtgcaaggtgtccaacaag
ggcctgcccagcagcatcgaaaagaccatcagcaaggccaagggccagcctcgcgagccccaggtgtacaccctgc
ctccctcccaggaagagatgaccaagaaccaggtgtccctgacctgcctggtgaagggcttctaccccagcgacat
cgccgtggagtgggagagcaacggccagcctgagaacaactacaagaccacccctcccgtgctggacagcgacggc
gcagcttcttcctgtacagccggctgaccgtggacaagagccggtggcaggaaggcaacgtctttagctgcagcgt
gatgcacgaggccctgcacaaccactacacccagaagagcctgagcctgtccctgggcaagatgttctgggtgctg
gtggtggtgggcggggtgctggcctgctacagcctgctggtgacagtggccttcatcatcttttgggtgcggagca
agcggagcagaggcggccacagcgactacatgaacatgacccccagacggcctggccccacccggaagcactacca
gccctacgccccacccagggactttgccgcctacagaagcaaacggggcagaaagaaactcctgtatatattcaaa
caaccatttatgagaccagtacaaactactcaagaggaagatggctgtagctgccgatttccagaagaagaagaag
gaggatgtgaactgcgggtgaagttcagcagaagcgccgacgcccctgcctaccagcagggccagaatcagctgta
caacgagctgaacctgggcagaagggaagagtacgacgtcctggataagcggagaggccgggaccctgagatgggc
ggcaagcctcggcggaagaacccccaggaaggcctgtataacgaactgcagaaagacaagatggccgaggcctaca
gcgagatcggcatgaagggcgagcggaggcggggcaagggccacgacggcctgtatcagggcctgtccaccgccac
caaggatacctacgacgccctgcacatgcaggccctgcccccaaggctcgagggcggcggagagggcagaggaagt
cttctaacatgcggtgacgtggaggagaatcccggccctaggATGGCAAGGCTGGGAAACTGCA
GCCTGACTTGGGCTGCCCTGATCATCCTGCTGCTCCCCGGAAGTC
TGGAGGAGTGCGGGCACATCAGTGTCTCAGCCCCCATCGTCCACC
TGGGGGATCCCATCACAGCCTCCTGCATCATCAAGCAGAACTGCA
GCCATCTGGACCCGGAGCCACAGATTCTGTGGAGACTGGGAGCAG
AGCTTCAGCCCGGGGGCAGGCAGCAGCGTCTGTCTGATGGGACCC
AGGAATCTATCATCACCCTGCCCCACCTCAACCACACTCAGGCCT
TTCTCTCCTGCTGCCTGAACTGGGGCAACAGCCTGCAGATCCTGG
ACCAGGTTGAGCTGCGCGCAGGCTACCCTCCAGCCATACCCCACA
ACCTCTCCTGCCTCATGAACCTCACAACCAGCAGCCTCATCTGCC
AGTGGGAGCCAGGACCTGAGACCCACCTACCCACCAGCTTCACTC
TGAAGAGTTTCAAGAGCCGGGGCAACTGTCAGACCCAAGGGGACT
CCATCCTGGACTGCGTGCCCAAGGACGGGCAGAGCCACTGCTGCA
TCCCACGCAAACACCTGCTGTTGTACCAGAATATGGGCATCTGGG
TGCAGGCAGAGAATGCGCTGGGGACCAGCATGTCCCCACAACTGT
GTCTTGATCCCATGGATGTTGTGAAACTGGAGCCCCCCATGCTGC
GGACCATGGACCCCAGCCCTGAAGCGGCCCCTCCCCAGGCAGGCT
GCCTACAGCTGTGCTGGGAGCCATGGCAGCCAGGCCTGCACATAA
ATCAGAAGTGTGAGCTGCGCCACAAGCCGCAGCGTGGAGAAGCCA
GCTGGGCACTGGTGGGCCCCCTCCCCTTGGAGGCCCTTCAGTATG
AGCTCTGCGGGCTCCTCCCAGCCACGGCCTACACCCTGCAGATAC
GCTGCATCCGCTGGCCCCTGCCTGGCCACTGGAGCGACTGGAGCC
CCAGCCTGGAGCTGAGAACTACCGAACGGGCCCCCACTGTCAGAC
TGGACACATGGTGGCGGCAGAGGCAGCTGGACCCCAGGACAGTGC
AGCTGTTCTGGAAGCCAGTGCCCCTGGAGGAAGACAGCGGACGGA
TCCAAGGTTATGTGGTTTCTTGGAGACCCTCAGGCCAGGCTGGGG
CCATCCTGCCCCTCTGCAACACCACAGAGCTCAGCTGCACCTTCC
ACCTGCCTTCAGAAGCCCAGGAGGTGGCCCTTGTGGCCTATAACT
CAGCCGGGACCTCTCGTCCCACTCCGGTGGTCTTCTCAGAAAGCA
GAGGCCCAGCTCTGACCAGACTCCATGCCATGGCCCGAGACCCTC
ACAGCCTCTGGGTAGGCTGGGAGCCCCCCAATCCATGGCCTCAGG
GCTATGTGATTGAGTGGGGCCTGGGCCCCCCCAGCGCGAGCAATA
GCAACAAGACCTGGAGGATGGAACAGAATGGGAGAGCCACGGGGT
TTCTGCTGAAGGAGAACATCAGGCCCTTTCAGCTCTATGAGATCA
TCGTGACTCCCTTGTACCAGGACACCATGGGACCCTCCCAGCATG
TCTATGCCTACTCTCAAGAAATGGCTCCCTCCCATGCCCCAGAGC
TGCATCTAAAGCACATTGGCAAGACCTGGGCACAGCTGGAGTGGG
TGCCTGAGCCCCCTGAGCTGGGGAAGAGCCCCCTTACCCACTACA
CCATCTTCTGGACCAACGCTCAGAACCAGTCCTTCTCCGCCATCC
TGAATGCCTCCTCCCGTGGCTTTGTCCTCCATGGCCTGGAGCCCG
CCAGTCTGTATCACATCCACCTCATGGCTGCCAGCCAGGCTGGGG
CCACCAACAGTACAGTCCTCACCCTGATGACCTTGACCCCAGAGG
GGTCGGAGCTACACATCATCCTGGGCCTGTTCGGCCTCCTGCTGT
TGCTCACCTGCCTCTGTGGAACTGCCTGGCTCTGTTGCAGCCCCA
ACAGGAAGAATCCCCTCTGGCCAAGTGTCCCAGACCCAGCTCACA
GCAGCCTGGGCTCCTGGGTGCCCACAATCATGGAGGAGGATGCCT
TCCAGCTGCCCGGCCTTGGCACGCCACCCATCACCAAGCTCACAG
TGCTGGAGGAGGATGAAAAGAAGCCGGTGCCCTGGGAGTCCCATA
ACAGCTCAGAGACCTGTGGCCTCCCCACTCTGGTCCAGACCTATG
TGCTCCAGGGGGACCCAAGAGCAGTTTCCACCCAGCCCCAATCCC
AGTCTGGCACCAGCGATCAGGTCCTTTATGGGCAGCTGCTGGGCA
GCCCCACAAGCCCAGGGCCAGGGCACTATCTCCGCTGTGACTCCA
CTCAGCCCCTCTTGGCGGGCCTCACCCCCAGCCCCAAGTCCTATG
AGAACCTCTGGTTCCAGGCCAGCCCCTTGGGGACCCTGGTAACCC
CAGCCCCAAGCCAGGAGGACGACTGTGTCTTTGGGCCACTGCTCA
ACTTCCCCCTCCTGCAGGGGATCCGGGTCCATGGGATGGAGGCGC
TGGGGAGCTTCTAGagcggccgcgtcgacaatcaacctctggattacaaaatttgtgaaagattgactggtattctt
aactatgttgctccttttacgctatgtggatacgctgctttaatgcctttgtatcatgctattgcttcccgtatggc
tttcattttctcctccttgtataaatcctggttgctgtctctttatgaggagttgtggcccgttgtcaggcaacgtg
gcgtggtgtgcactgtgtttgctgacgcaacccccactggttggggcattgccaccacctgtcagctcctttccggg
actttgctttccccctccctattgccacggcggaactcatcgccgcctgccttgcccgctgctggacaggggctcgg
ctgttgggcactgacaattccgtggtgttgtcggggaagctgacgtcctttccttggctgctcgcctgtgttgccac
ctggattctgcgcgggacgtccttctgctacgtcccttcggccctcaatccagcggaccttccttcccgcggcctgc
tgccggctctgcggcctcttccgcgtcttcgccttcgccctcagacgagtcggatctccctttgggccgcctccccg
cctggaattcgagctcggtacctttaagaccaatgacttacaaggcagctgtagatcttagccactttttaaaagaa
aaggggggactggaagggctaattcactcccaacgaagacaagatctgctttttgcttgtactgggtctctctggtt
agaccagatctgagcctgggagctctctggctaactagggaacccactgcttaagcctcaataaagcttgccttgag
tgcttcaagtagtgtgtgcccgtctgttgtgtgactctggtaactagagatccctcagacccttttagtcagtgtgg
aaaatctctagcagtagtagttcatgtcatcttattattcagtatttataacttgcaaagaaatgaatatcagagag
tgagaggaacttgtttattgcagcttataatggttacaaataaagcaatagcatcacaaatttcacaaataaagcat
ttttttcactgcattctagttgtggtttgtccaaactcatcaatgtatcttatcatgtctggctctagcttcccgca
ccctaactccgcccagttccgcccattctccgccccatggctgactaattttttttatttatgcagaggccgaggcc
gcctcggcctctgagctattccagaagtagtgaggaggcttttttggaggcctagctagggacgtacccaattcgcc
ctatagtgagtcgtattacgcgcgctcactggccgtcgttttacaacgtcgtgactgggaaaaccctggcgttaccc
aacttaatcgccttgcagcacatccccctttcgccagctggcgtaatagcgaagaggcccgcaccgatcgcccttcc
caacagttgcgcagcctgaatggcgaatgggacgcgccctgtagcggcgcattaagcgcggcgggtgtggtggttac
gcgcagcgtgaccgctacacttgccagcgccctagcgcccgctcctttcgctttcttcccttcctttctcgccacgt
ctcgcggctttccccgtcaagctctaaatcgggggctccctttagggttccgatttagtgctttacggcacctcgac
cccaaaaaacttgattagggtgatggttcacgtagtgggccatcgccctgatagacggtttttcgccctttgacgtt
ggagtccacgttctttaatagtggactcttgttccaaactggaacaacactcaaccctatctcggtctattcttttg
atttataagggattttgccgatttcggcctattggttaaaaaatgagctgatttaacaaaaatttaacgcgaatttt
aacaaaatattaacgcttacaatttaggtggcacttttcggggaaatgtgcgcggaacccctatttgtttatttttc
taaatacattcaaatatgtatccgctcatgagacaataaccctgataaatgcttcaataatattgaaaaaggaagag
tatgagtattcaacatttccgtgtcgcccttattcccttttttgcggcattttgccttcctgtttttgctcacccag
aaacgctggtgaaagtaaaagatgctgaagatcagttgggtgcacgagtgggttacatcgaactggatctcaacagc
ggtaagatccttgagagttttcgccccgaagaacgttttccaatgatgagcacttttaaagttctgctatgtggcgc
ggtattatcccgtattgacgccgggcaagagcaactcggtcgccgcatacactattctcagaatgacttggttgagt
actcaccagtcacagaaaagcatcttacggatggcatgacagtaagagaattatgcagtgctgccataaccatgagt
gataacactgcggccaacttacttctgacaacgatcggaggaccgaaggagctaaccgcttttttgcacaacatggg
ggatcatgtaactcgccttgatcgttgggaaccggagctgaatgaagccataccaaacgacgagcgtgacaccacga
tgcctgtagcaatggcaacaacgttgcgcaaactattaactggcgaactacttactctagcttcccggcaacaatta
atagactggatggaggcggataaagttgcaggaccacttctgcgctcggcccttccggctggctggtttattgctga
taaatctggagccggtgagcgtgggtctcgcggtatcattgcagcactggggccagatggtaagccctcccgtatcg
tagttatctacacgacggggagtcaggcaactatggatgaacgaaatagacagatcgctgagataggtgcctcactg
attaagcattggtaactgtcagaccaagtttactcatatatactttagattgatttaaaacttcatttttaatttaa
aaggatctaggtgaagatcctttttgataatctcatgaccaaaatcccttaacgtgagttttcgttccactgagcgt
tcagaccccgtagaaaagatcaaaggatcttcttgagatcctttttttctgcgcgtaatctgctgcttgcaaacaaa
aaaaccaccgctaccagcggtggtttgtttgccggatcaagagctaccaactctttttccgaaggtaactggcttca
gcagagcgcagataccaaatactgttcttctagtgtagccgtagttaggccaccacttcaagaactctgtagcaccg
cctacatacctcgctctgctaatcctgttaccagtggctgctgccagtggcgataagtcgtgtcttaccgggttgga
ctcaagacgatagttaccggataaggcgcagcggtcgggctgaacggggggttcgtgcacacagcccagcttggagc
gaacgacctacaccgaactgagatacctacagcgtgagctatgagaaagcgccacgcttcccgaagggagaaaggcg
gacaggtatccggtaagcggcagggtcggaacaggagagcgcacgagggagcttccagggggaaacgcctggtatct
ttatagtcctgtcgggtttcgccacctctgacttgagcgtcgatttttgtgatgctcgtcaggggggggagcctatg
gaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgag
cgcagcgagtcagtgagcgaggaagcggaagagcgcccaatacgcaaaccgcctctccccgcgcgttggccgattca
ttaatgcagctggcacgacaggtttcccgactggaaagcgggcagtgagcgcaacgcaattaatgtgagttagctca
ctcattaggcaccccaggctttacactttatgcttccggctcgtatgttgtgtggaattgtgagcggataacaattt
cacacaggaaacagctatgaccatgattacgccaagcgcgcaattaaccctcactaaagggaacaaaagctggagct
gcaagcttaatgtagtcttatgcaatactcttgtagtcttgcaacatggtaacgatgagttagcaacatgccttaca
aggagagaaaaagcaccgtgcatgccgattggtggaagtaaggtggtacgatcgtgccttattaggaaggcaacaga
cgggtctgacatggattggacgaaccactgaattgccgcattgcagagatattgtatttaagtgcctagctcgatac
ataaac
pTRPE CD20CAR-CSF3Rv4 (SEQ ID NO: 4);
(CSF3Rv4 (SEQ ID NO: 14) comprises
nucleotides 5562-7913 (double underlined):
gggtctctctggttagaccagatctgagcctgggagctctctggctaactagggaacccactgcttaagcctcaata
aagcttgccttgagtgcttcaagtagtgtgtgcccgtctgttgtgtgactctggtaactagagatccctcagaccct
tttagtcagtgtggaaaatctctagcagtggcgcccgaacagggacttgaaagcgaaagggaaaccagagagctctc
gtcgacgcaggactcggcttgctgaagcgcgcacggcaagaggcgaggggggcgactggtgagtacgccaaaaattt
tgactagcggaggctagaaggagagagatgggtgcgagagcgtcagtattaagcgggggagaattagatcgcgatgg
gaaaaaattcggttaaggccagggggaaagaaaaaatataaattaaaacatatagtatgggcaagcagggagctaga
acgattcgcagttaatcctggcctgttagaaacatcagaaggctgtagacaaatactgggacagctacaaccatcc
cttcagacaggatcagaagaacttagatcattatataatacagtagcaaccctctattgtgtgcatcaaaggatag
agataaaagacaccaaggaagctttagacaagatagaggaagagcaaaacaaaagtaagaccaccgcacagcaagc
ggccgctgatcttcagacctggaggaggagatatgagggacaattggagaagtgaattatataaatataaagtagt
aaaaattgaaccattaggagtagcacccaccaaggcaaagagaagagtggtgcagagagaaaaaagagcagtggga
ataggagctttgttccttgggttcttgggagcagcaggaagcactatgggcgcagcgtcaatgacgctgacggtac
aggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaa
cagctcctggggatttggggttgctctggaaaactcatttgcaccactgctgtgccttggaatgctagttggagta
ataaatctctggaacagatttggaatcacacgacctggatggagtgggacagagaaattaacaattacacaagctta
aatacactccttaattgaagaatcgcaaaaccagcaagaaaagaatgaacaagaattattggaattagataaatggg
caagtttgtggaattggtttaacataacaaattggctgtggtatataaaattattcataatgatagtaggaggcttg
gtaggtttaagaatagtttttgctgtactttctatagtgaatagagttaggcagggatattcaccattatcgtttca
gacccacctcccaaccccgaggggacccgacaggcccgaaggaatagaagaagaaggtggagagagagacagagaca
gatccattcgattagtgaacggatctcgacggtatcgattagactgtagcccaggaatatggcagctagattgtaca
catttagaaggaaaagttatcttggtagcagttcatgtagccagtggatatatagaagcagaagtaattccagcaga
gacagggcaagaaacagcatacttcctcttaaaattagcaggaagatggccagtaaaaacagtacatacagacaatg
gcagcaatttcaccagtactacagttaaggccgcctgttggtgggcggggatcaagcaggaatttggcattccctac
aatccccaaagtcaaggagtaatagaatctatgaataaagaattaaagaaaattataggacaggtaagagatcaggc
tgaacatcttaagacagcagtacaaatggcagtattcatccacaattttaaaagaaaaggggggattggggggtaca
gtgcaggggaaagaatagtagacataatagcaacagacatacaaactaaagaattacaaaaacaaattacaaaaatt
caaaattttcgggtttattacagggacagcagagatccagtttggctgcatacgcgtcgtgaggctccggtgcccgt
cagtgggcagagcgcacatcgcccacagtccccgagaagttggggggaggggtcggcaattgaaccggtgcctagag
aaggtggcgcggggtaaactgggaaagtgatgtcgtgtactggctccgcctttttcccgagggtgggggagaaccgt
atataagtgcagtagtcgccgtgaacgttctttttcgcaacgggtttgccgccagaacacaggtaagtgccgtgtgt
ggttcccgcgggcctggcctctttacgggttatggcccttgcgtgccttgaattacttccacctggctgcagtacgt
gattcttgatcccgagcttcgggttggaagtggggggagagttcgaggccttgcgcttaaggagccccttcgcctcg
tgcttgagttgaggcctggcctgggcgctggggccgccgcgtgcgaatctggtggcaccttcgcgcctgtctcgctg
ctttcgataagtctctagccatttaaaatttttgatgacctgctgcgacgctttttttctggcaagatagtcttgta
aatgcgggccaagatctgcacactggtatttcggtttttggggccgcgggggcgacggggcccgtgcgtcccagcgc
acatgttcggcgaggcggggcctgcgagcgcggccaccgagaatcggacgggggtagtctcaagctggccggcctgc
tctggtgcctggcctcgcgccgccgtgtatcgccccgccctgggcggcaaggctggcccggtcggcaccagttgcgt
gagcggaaagatggccgcttcccggccctgctgcagggagctcaaaatggaggacgcggcgctcgggagagcggggg
gtgagtcacccacacaaaggaaaagggcctttccgtcctcagccgtcgcttcatgtgactccactgagtaccgggcg
ccgtccaggcacctcgattagttctcgtgcttttggagtacgtcgtctttaggttggggggaggggttttatgcgat
ggagtttccccacactgagtgggtggagactgaagttaggccagcttggcacttgatgtaattctccttggaatttg
ccctttttgagtttggatcttggttcattctcaagcctcagacagtggttcaaagtttttttcttccatttcaggtgt
cgtgagctagctctagagccgccatggagacagacacactcctgctatgggtgctgctgctctgggttccaggttcca
caggtgacattgtgctgacccaatctccagctatcctgtctgcatctccaggggagaaggtcacaatgacttgcaggg
ccagctcaagtgtaaattacatggactggtaccagaagaagccaggatcctcccccaaaccctggatttatgccacat
ccaacctggcttctggagtccctgctcgcttcagtggcagtgggtctgggacctcttactctctcacaatcagcagag
tggaggctgaagatgctgccacttattactgccagcagtggagttttaatccacccacgttcggaggggggaccaagc
tggaaaaaaaggcagtactagcggtggtggctccgggggcggttccggtgggggcggcagcagcgaggtgcagctgca
gcagtctggggctgagctggtgaagcctggggcctcagtgaagatgtcctgcaaggcttctggctacacatttaccag
ttacaatatgcactgggtaaagcagacacctggacagggcctggaatggattggagctatttatccaggaaatggtga
tacttcctacaatcagaagttcaaaggcaaggccacattgactgcagacaaatcctccagcacagcctacatgcagtc
cagcagcctgacatctgaggactctgcggactattactgtgcaagatctaattattacggtagtagctactggttctt
cgatgtctggggcgcagggaccacggtcaccgtctcctcactcgacgaatctaagtacggaccgccctgccccccttg
ccctgcccccgagttcctgggcggacccagcgtgttcctgttcccccccaagcccaaggacaccctgatgatcagccg
gacccccgaggtgacctgcgtggtggtggacgtgagccaggaagatcccgaggtccagttcaattggtacgtggacgg
cgtggaagtgcacaacgccaagaccaagcccagagaggaacagttcaacagcacctaccgggtggtgtctgtgctgac
cgtgctgcaccaggactggctgaacggcaaagaatacaagtgcaaggtgtccaacaagggcctgcccagcagcatcga
aaagaccatcagcaaggccaagggccagcctcgcgagccccaggtgtacaccctgcctccctcccaggaagagatgac
caagaaccaggtgtccctgacctgcctggtgaagggcttctaccccagcgacatcgccgtggagtgggagagcaacgg
ccagcctgagaacaactacaagaccacccctcccgtgctggacagcgacggcagcttcttcctgtacagccggctgac
cgtggacaagagccggtggcaggaaggcaacgtctttagctgcagcgtgatgcacgaggccctgcacaaccactacac
ccagaagagcctgagcctgtccctgggcaagatgttctgggtgctggtggtggtgggcggggtgctggcctgctacag
cctgctggtgacagtggccttcatcatcttttgggtgcggagcaagcggagcagaggcggccacagcgactacatgaa
catgacccccagacggcctggccccacccggaagcactaccagccctacgccccacccagggactttgccgcctacag
aagcaaacggggcagaaagaaactcctgtatatattcaaacaaccatttatgagaccagtacaaactactcaagagga
agatggctgtagctgccgatttccagaagaagaagaaggaggatgtgaactgcgggtgaagttcagcagaagcgccga
cgcccctgcctaccagcagggccagaatcagctgtacaacgagctgaacctgggcagaagggaagagtacgacgtcct
ggataagcggagaggccgggaccctgagatgggcggcaagcctcggcggaagaacccccaggaaggcctgtataacga
actgcagaaagacaagatggccgaggcctacagcgagatcggcatgaagggcgagcggaggcggggcaagggccacga
cggcctgtatcagggcctgtccaccgccaccaaggatacctacgacgccctgcacatgcaggccctgcccccaaggct
cgagggcggcggagagggcagaggaagtcttctaacatgcggtgacgtggaggagaatcccggccctagg
ATGGCAAGGCTGGGAAACTGCAGCCTGACTTGGGCTGCCCTGATCATCCTGCTGCTCCCCGGAAGTCTG
GAGGAGTGCGGGCACATCAGTGTCTCAGCCCCCATCGTCCACCTG
GGGGATCCCATCACAGCCTCCTGCATCATCAAGCAGAACTGCAGC
CATCTGGACCCGGAGCCACAGATTCTGTGGAGACTGGGAGCAGAG
CTTCAGCCCGGGGGCAGGCAGCAGCGTCTGTCTGATGGGACCCAG
GAATCTATCATCACCCTGCCCCACCTCAACCACACTCAGGCCTTT
CTCTCCTGCTGCCTGAACTGGGGCAACAGCCTGCAGATCCTGGAC
CAGGTTGAGCTGCGCGCAGGCTACCCTCCAGCCATACCCCACAAC
CTCTCCTGCCTCATGAACCTCACAACCAGCAGCCTCATCTGCCAG
TGGGAGCCAGGACCTGAGACCCACCTACCCACCAGCTTCACTCTG
AAGAGTTTCAAGAGCCGGGGCAACTGTCAGACCCAAGGGGACTCC
ATCCTGGACTGCGTGCCCAAGGACGGGCAGAGCCACTGCTGCATC
CCACGCAAACACCTGCTGTTGTACCAGAATATGGGCATCTGGGTG
CAGGCAGAGAATGCGCTGGGGACCAGCATGTCCCCACAACTGTGT
CTTGATCCCATGGATGTTGTGAAACTGGAGCCCCCCATGCTGCGG
ACCATGGACCCCAGCCCTGAAGCGGCCCCTCCCCAGGCAGGCTGC
CTACAGCTGTGCTGGGAGCCATGGCAGCCAGGCCTGCACATAAAT
CAGAAGTGTGAGCTGCGCCACAAGCCGCAGCGTGGAGAAGCCAGC
TGGGCACTGGTGGGCCCCCTCCCCTTGGAGGCCCTTCAGTATGAG
CTCTGCGGGCTCCTCCCAGCCACGGCCTACACCCTGCAGATACGC
TGCATCCGCTGGCCCCTGCCTGGCCACTGGAGCGACTGGAGCCCC
AGCCTGGAGCTGAGAACTACCGAACGGGCCCCCACTGTCAGACTG
GACACATGGTGGCGGCAGAGGCAGCTGGACCCCAGGACAGTGCAG
CTGTTCTGGAAGCCAGTGCCCCTGGAGGAAGACAGCGGACGGATC
CAAGGTTATGTGGTTTCTTGGAGACCCTCAGGCCAGGCTGGGGCC
ATCCTGCCCCTCTGCAACACCACAGAGCTCAGCTGCACCTTCCAC
CTGCCTTCAGAAGCCCAGGAGGTGGCCCTTGTGGCCTATAACTCA
GCCGGGACCTCTCGTCCCACTCCGGTGGTCTTCTCAGAAAGCAGA
GGCCCAGCTCTGACCAGACTCCATGCCATGGCCCGAGACCCTCAC
AGCCTCTGGGTAGGCTGGGAGCCCCCCAATCCATGGCCTCAGGGC
TATGTGATTGAGTGGGGCCTGGGCCCCCCCAGCGCGAGCAATAGC
AACAAGACCTGGAGGATGGAACAGAATGGGAGAGCCACGGGGTTT
CTGCTGAAGGAGAACATCAGGCCCTTTCAGCTCTATGAGATCATC
GTGACTCCCTTGTACCAGGACACCATGGGACCCTCCCAGCATGTC
TATGCCTACTCTCAAGAAATGGCTCCCTCCCATGCCCCAGAGCTG
CATCTAAAGCACATTGGCAAGACCTGGGCACAGCTGGAGTGGGTG
CCTGAGCCCCCTGAGCTGGGGAAGAGCCCCCTTACCCACTACACC
ATCTTCTGGACCAACGCTCAGAACCAGTCCTTCTCCGCCATCCTG
AATGCCTCCTCCCGTGGCTTTGTCCTCCATGGCCTGGAGCCCGCC
AGTCTGTATCACATCCACCTCATGGCTGCCAGCCAGGCTGGGGCC
ACCAACAGTACAGTCCTCACCCTGATGACCTTGACCCCAGAGGGG
TCGGAGCTACACATCATCCTGGGCCTGTTCGGCCTCCTGCTGTTG
CTCACCTGCCTCTGTGGAACTGCCTGGCTCTGTTGCAGCCCCAAC
AGGAAGAATCCCCTCTGGCCAAGTGTCCCAGACCCAGCTCACAGC
AGCCTGGGCTCCTGGGTGCCCACAATCATGGAGGAGGATGCCTTC
CAGCTGCCCGGCCTTGGCACGCCACCCATCACCAAGCTCACAGTG
CTGGAGGAGGATGAAAAGAAGCCGGTGCCCTGGGAGTCCCATAAC
AGCTCAGAGACCTGTGGCCTCCCCACTCTGGTCCAGACCTATGTG
CTCCAGGGGGACCCAAGAGCAGTTTCCACCCAGCCCCAATCCCAG
TCTGGCACCAGCGATCAGGCTGGGCCTCCCAGGCGATCTGCATAC
TTTAAGGACCAGATCATGCTCCATCCAGCCCCACCCAATGGCCTT
TTGTGCTTGTTTCCTATAACTTCAGTATTGTAAagcggccgcgtcgacaatcaacctctggattacaaaatttgtga
aagattgactggtattcttaactatgttgctccttttacgctatgtggatacgctgctttaatgcctttgtatcatg
ctattgcttcccgtatggctttcattttctcctccttgtataaatcctggttgctgtctctttatgaggagttgtgg
cccgttgtcaggcaacgtggcgtggtgtgcactgtgtttgctgacgcaacccccactggttggggcattgccaccac
ctgtcagctcctttccgggactttcgctttccccctccctattgccacggcggaactcatcgccgcctgccttgccc
gctgctggacaggggctcggctgttgggcactgacaattccgtggtgttgtcggggaagctgacgtcctttccttggc
tgctcgcctgtgttgccacctggattctgcgcgggacgtccttctgctacgtcccttcggccctcaatccagcggacc
ttccttcccgcggcctgctgccggctctgcggcctcttccgcgtcttcgccttcgccctcagacgagtcggatctccc
tttgggccgcctccccgcctggaattcgagctcggtacctttaagaccaatgacttacaaggcagctgtagatcttag
ccactttttaaaagaaaaggggggactggaagggctaattcactcccaacgaagacaagatctgctttttgcttgtac
tgggtctctctggttagaccagatctgagcctgggagctctctggctaactagggaacccactgcttaagcctcaata
aagcttgccttgagtgcttcaagtagtgtgtgcccgtctgttgtgtgactctggtaactagagatccctcagaccctt
ttagtcagtgtggaaaatctctagcagtagtagttcatgtcatcttattattcagtatttataacttgcaaagaaatg
aatatcagagagtgagaggaacttgtttattgcagcttataatggttacaaataaagcaatagcatcacaaatttcac
aaataaagcatttttttcactgcattctagttgtggtttgtccaaactcatcaatgtatcttatcatgtctggctcta
gctatcccgcccctaactccgcccagttccgcccattctccgccccatggctgactaattttttttatttatgcagag
gccgaggccgcctcggcctctgagctattccagaagtagtgaggaggcttttttggaggcctgctagggacgtaccca
aattcgccctatagtgagtcgtattacgcgcgctcactggccgtcgttttacaacgtcgtgactgggaaaaccctggc
gttacccaacttaatcgccttgcagcacatccccctttcgccagctggcgtaatagcgaagaggcccgcaccgatcgc
ccttcccaacagttgcgcagcctgaatggcgaatgggacgcgccctgtagcggcgcattaagcgcggcgggtgtggtg
gttacgcgcagcgtgaccgctacacttgccagcgccctagcgcccgctcctttcgctttcttcccttcctttctcgcc
acgttcgccggctttccccgtcaagctctaaatcgggggctccctttagggttccgatttagtgctttacggcacctc
gaccccaaaaaacttgattagggtgatggttcacgtagtgggccatcgccctgatagacggtttttcgccctttgacg
ttggagtccacgttctttaatagtggactcttgttccaaactggaacaacactcaaccctatctcggtctattctttt
gatttataagggattttgccgatttcggcctattggttaaaaaatgagctgatttaacaaaaatttaacgcgaatttt
aacaaaatattaacgcttacaatttaggtggcacttttcggggaaatgtgcgcggaacccctatttgtttatttttct
aaatacattcaaatatgtatccgctcatgagacaataaccctgataaatgcttcaataatattgaaaaaggaagagta
tgagtattcaacatttccgtgtcgcccttattcccttttttgcggcattttgccttcctgtttttgctcacccagaaa
cgctggtgaaagtaaaagatgctgaagatcagttgggtgcacgagtgggttacatcgaactggatctcaacagcggta
agatccttgagagttttcgccccgaagaacgttttccaatgatgagcacttttaaagttctgctatgtggcgcggta
ttatcccgtattgacgccgggcaagagcaactcggtcgccgcatacactattctcagaatgacttggttgagtactc
accagtcacagaaaagcatcttacggatggcatgacagtaagagaattatgcagtgctgccataaccatgagtgataa
cactgcggccaacttacttctgacaacgatcggaggaccgaaggagctaaccgcttttttgcacaacatgggggatca
catgtaactcgccttgatcgttgggaaccggagctgaatgaagccataccaaacgacgagcgtgacaccacgatgcc
tgtagcaatggcaacaacgttgcgcaaactattaactggcgaactacttactctagcttcccggcaacaattaata
gactggatggaggcggataaagttgcaggaccacttctgcgctcggcccttccggctggctggtttattgctgataa
atctggagccggtgagcgtgggtctcgcggtatcattgcagcactggggccagatggtaagccctcccgtatcgtag
ttatctacacgacggggagtcaggcaactatggatgaacgaaatagacagatcgctgagataggtgcctcactgatta
agcattggtaactgtcagaccaagtttactcatatatactttagattgatttaaaacttcatttttaatttaaaagga
tctaggtgaagatcctttttgataatctcatgaccaaaatcccttaacgtgagttttcgttccactgagcgtcagac
cccgtagaaaagatcaaaggatcttcttgagatcctttttttctgcgcgtaatctgctgcttgcaaacaaaaaaacc
accgctaccagcggtggtttgtttgccggatcaagagctaccaactctttttccgaaggtaactggcttcagcagagc
gcagataccaaatactgttcttctagtgtagccgtagttaggccaccacttcaagaactctgtagcaccgcctacata
cctcgctctgctaatcctgttaccagtggctgctgccagtggcgataagtcgtgtcttaccgggttggactcaagacg
atagttaccggataaggcgcagcggtcgggctgaacggggggttcgtgcacacagcccagcttggagcgaacgaccta
caccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgt
cgggtttcgccacctctgacttgagcgtcgatttttgtgatgctcgtcaggggggcggagcctatggaaaaacgccag
caacgcggcctttttacggttcctggccttttgctggccttttgctcacatgttctttcctgcgttatcccctgattc
tgtggataaccgtattaccgcctttgagtgagctgataccgctcgccgcagccgaacgaccgagcgcagcgagtcagt
gagcgaggaagcggaagagcgcccaatacgcaaaccgcctctccccgcgcgttggccgattcattaatgcagctggca
cgacaggtttcccgactggaaagcgggcagtgagcgcaacgcaattaatgtgagttagctcactcattaggcacccca
ggctttacactttatgcttccggctcgtatgttgtgtggaattgtgagcggataacaatttcacacaggaaacagcta
tatgaccatgattacgccaagcgcgcaattaaccctcactaaagggaacaaaagctggagctgcaagcttaatgtagt
cttatgcaatactcttgtagtcttgcaacatggtaacgatgagttagcaacatgccttacaaggagagaaaaagcacc
gtgcatgccgattggtggaagtaaggtggtacgatcgtgccttattaggaaggcaacagacgggtctgacatggattg
gacgaaccactgaattgccgcattgcagagatattgtatttaagtgcctagctcgatacataaac
CAR19-CSF3R d715 vector (SEQ ID NO: 5); (CAR19-CSF3R d715 (SEQ ID NO: 6)
comprises nucleotides 3324 . . . 7007):
      1 gggtctctct ggttagacca gatctgagcc tgggagctct ctggctaact agggaaccca
    61 ctgcttaagc ctcaataaag cttgccttga gtgcttcaag tagtgtgtgc ccgtctgttg
  121 tgtgactctg gtaactagag atccctcaga cccttttagt cagtgtggaa aatctctagc
  181 agtggcgccc gaacagggac ttgaaagcga aagggaaacc agaggagctc tctcgacgca
  241 ggactcggct tgctgaagcg cgcacggcaa gaggcgaggg goggcgactg gtgagtacgc
  301 caaaaatttt gactagcgga ggctagaagg agagagatgg gtgcgagagc gtcagtatta
  361 agcgggggag aattagatcg cgatgggaaa aaattcggtt aaggccaggg ggaaagaaaa
  421 aatataaatt aaaacatata gtatgggcaa gcagggagct agaacgattc gcagttaatc
  481 ctggcctgtt agaaacatca gaaggctgta gacaaatact gggacagcta caaccatccc
  541 ttcagacagg atcagaagaa cttagatcat tatataatac agtagcaacc ctctattgtg
  601 tgcatcaaag gatagagata aaagacacca aggaagcttt agacaagata gaggaagagc
  661 aaaacaaaag taagaccacc gcacagcaag cggccgctga tottcagacc tggaggagga
  721 gatatgaggg acaattggag aagtgaatta tataaatata aagtagtaaa aattgaacca
  781 ttaggagtag cacccaccaa ggcaaagaga agagtggtgc agagagaaaa aagagcagtg
  841 ggaataggag ctttgttcct tgggttcttg ggagcagcag gaagcactat gggcgcagcg
  901 tcaatgacgc tgacggtaca ggccagacaa ttattgtctg gtatagtgca gcagcagaac
  961 aatttgctga gggctattga ggcgcaacag catctgttgc aactcacagt ctggggcatc
1021 aagcagctcc aggcaagaat cctggctgtg gaaagatacc taaaggatca acagctcctg
1081 gggatttggg gttgctctgg aaaactcatt tgcaccactg ctgtgccttg gaatgctagt
1141 tggagtaata aatctctgga acagatttgg aatcacacga cctggatgga gtgggacaga
1201 gaaattaaca attacacaag cttaatacac tccttaattg aagaatcgca aaaccagcaa
1261 gaaaagaatg aacaagaatt attggaatta gataaatggg caagtttgtg gaattggttt
1321 aacataacaa attggctgtg gtatataaaa ttattcataa tgatagtagg aggcttggta
1381 ggtttaagaa tagtttttgc tgtactttct atagtgaata gagttaggca gggatattca
1441 ccattategt ttcagaccca cctoccaacc ccgaggggac ccgacaggcc cgaaggaata
1501 gaagaagaag gtggagagag agacagagac agatccattc gattagtgaa cggatctcga
1561 cggtatcgat tagactgtag cccaggaata tggcagctag attgtacaca tttagaagga
1621 aaagttatct tggtagcagt tcatgtagcc agtggatata tagaagcaga agtaattcca
1681 gcagagacag ggcaagaaac agcatacttc ctcttaaaat tagcaggaag atggccagta
1741 aaaacagtac atacagacaa tggcagcaat ttcaccagta ctacagttaa ggccgcctgt
1801 tggtgggcgg ggatcaagca ggaatttggc attccctaca atccccaaag tcaaggagta
1861 atagaatcta tgaataaaga attaaagaaa attataggac aggtaagaga tcaggctgaa
1921 catcttaaga cagcagtaca aatggcagta ttcatccaca attttaaaag aaaagggggg
1981 attggggggt acagtgcagg ggaaagaata gtagacataa tagcaacaga catacaaact
2041 aaagaattac aaaaacaaat tacaaaaatt caaaattttc gggtttatta cagggacagc
2101 agagatccag tttggctgca tacgcgtcgt gaggctccgg tgcccgtcag tgggcagagc
2161 gcacategcc cacagtcccc gagaagttgg ggggaggggt cggcaattga accggtgcct
2221 agagaaggtg gcgcggggta aactgggaaa gtgatgtcgt gtactggctc cgcctttttc
2281 ccgagggtgg gggagaaccg tatataagtg cagtagtcgc cgtgaacgtt ctttttcgca
2341 acgggtttgc cgccagaaca caggtaagtg ccgtgtgtgg ttcccgcggg cctggcctct
2401 ttacgggtta tggcccttgc gtgccttgaa ttacttccac ctggctgcag tacgtgattc
2461 ttgatcccga gcttcgggtt ggaagtgggt gggagagttc gaggccttgc gcttaaggag
2521 ccccttcgcc tegtgcttga gttgaggcct ggcctgggcg ctggggccgc cgcgtgcgaa
2581 tctggtggca cettegegcc tgtctogctg ctttcgataa gtctctagcc atttaaaatt
2641 tttgatgacc tgctgcgacg ctttttttct ggcaagatag tcttgtaaat gcgggccaag
2701 atctgcacac tggtatttg gtttttgggg ccgcgggegg cgacggggcc cgtgcgtccc
2761 agcgcacatg ttcggcgagg cggggcctgc gagcgcggcc accgagaatc ggacgggggt
2821 agtctcaagc tggccggcct gctctggtgc ctggcctege gccgccgtgt ategccccgc
2881 cctgggeggc aaggctggcc cggtcggcac cagttgcgtg agcggaaaga tggccgcttc
2941 ccggccctgc tgcagggagc tcaaaatgga ggacgcggcg ctcgggagag cgggcgggtg
3001 agtcacccac acaaaggaaa agggcctttc cgtcctcage cgtcgcttca tgtgactcca
3061 ctgagtaccg ggegccgtcc aggcacctcg attagttctc gtgcttttgg agtacgtcgt
3121 ctttaggttg gggggagggg ttttatgcga tggagtttcc ccacactgag tgggtggaga
3181 ctgaagttag gccagcttgg cacttgatgt aattctcctt ggaatttgcc ctttttgagt
3241 ttggatcttg gttcattctc aagcctcaga cagtggttca aagttttttt cttccatttc
3301 aggtgtcgtg agctagctct aga ATGGCAC TGCCAGTGAC CGCCCTGCTG CTGCCTCTGG
3361 CCCTGCTGCT GCACGCAGCA AGGCCAGACA TCCAGATGAC CCAGACCACA AGCTCCCTGA
3421 GCGCCTCCCT GGGCGACAGA GTGACAATCT CCTGCAGGGC CTCTCAGGAT ATCAGCAAGT
3481 ATCTGAACTG GTACCAGCAG AAGCCCGATG GCACCGTGAA GCTGCTGATC TATCACACAT
3541 CCCGGCTGCA CTCTGGCGTG CCTAGCAGAT TCTCTGGAAG CGGATCCGGA ACCGACTACT
3601 CCCTGACAAT CTCTAACCTG GAGCAGGAGG ATATCGCCAC CTATTTCTGC CAGCAGGGCA
3661 ATACCCTGCC TTACACATTT GGCGGCGGCA CCAAGCTGGA GATCACAGGC GGCGGCGGCT
3721 CTGGAGGAGG AGGAAGCGGA GGAGGAGGAT CCGAGGTGAA GCTGCAGGAG AGCGGACCAG
3781 GACTGGTGGC ACCTTCTCAG AGCCTGTCCG TGACATGCAC CGTGAGCGGC GTGTCCCTGC
3841 CAGACTACGG CGTGTCCTGG ATCAGGCAGC CACCTAGGAA GGGCCTGGAG TGGCTGGGCG
3901 TGATCTGGGG CTCTGAGACC ACATACTATA ATAGCGCCCT GAAGTCCAGA CTGACCATCA
3961 TCAAGGACAA CTCTAAGAGC CAGGTGTTCC TGAAGATGAA TTCCCTGCAG ACAGACGATA
4021 CCGCCATCTA CTATTGCGCC AAGCACTACT ATTACGGCGG CTCCTATGCC ATGGATTACT
4081 GGGGCCAGGG CACATCTGTG ACCGTGTCTA GCACCACAAC CCCTGCACCA AGGCCACCAA
4141 CCCCAGCACC TACAATCGCA TCTCAGCCAC TGAGCCTGCG CCCCGAGGCC TGTCGGCCTG
4201 CAGCAGGAGG AGCAGTGCAC ACCAGGGGCC TGGACTTTGC CTGCGATATC TACATCTGGG
4261 CACCTCTGGC AGGAACCTGT GGCGTGCTGC TGCTGAGCCT GGTCATCACC CTGTATTGCA
4321 AGAGAGGCAG GAAGAAGCTG CTGTACATCT TCAAGCAGCC CTTCATGAGG CCCGTGCAGA
4381 CAACCCAGGA GGAGGACGGC TGCTCCTGTC GCTTCCCTGA AGAGGAGGAG GGCGGCTGTG
4441 AGCTGAGAGT GAAGTTTTCC AGGTCTGCCG ATGCCCCAGC CTATAAGCAG GGCCAGAACC
4501 AGCTGTACAA CGAGCTGAAT CTGGGCCGGA GAGAGGAGTA CGACGTGCTG GATAAGAGGA
4561 GGGGAAGGGA CCCAGAGATG GGAGGCAAGC CACGGAGAAA GAACCCCCAG GAGGGCCTGT
4621 ATAATGAGCT GCAGAAGGAC AAGATGGCCG AGGCCTACAG CGAGATCGGC ATGAAGGGAG
4681 AGAGGCGCCG GGGCAAGGGA CACGATGGCC TGTATCAGGG CCTGTCCACA GCCACCAAGG
4741 ACACCTACGA TGCACTGCAC ATGCAGGCCC TGCCTCCACG CACCAGCGGA TCCGGCGCCA
4801 CAAATTTCAG CCTGCTGAAG CAGGCAGGCG ATGTGGAGGA GAACCCAGGA CCACCTAGAA
4861 TGGCAAGGCT GGGCAATTGC TCTCTGACCT GGGCCGCCCT GATCATCCTG CTGCTGCCTG
4921 GCAGCCTGGA GGAGTGTGGC CACATCTCTG TGAGCGCCCC AATCGTGCAC CTGGGCGACC
4981 CCATCACAGC CTCCTGCATC ATCAAGCAGA ACTGTTCTCA CCTGGATCCA GAGCCCCAGA
5041 TCCTGTGGAG GCTGGGAGCA GAGCTGCAGC CAGGAGGCCG CCAGCAGCGG CTGTCTGACG
5101 GCACCCAGGA GAGCATCATC ACACTGCCCC ACCTGAATCA CACCCAGGCC TTTCTGAGCT
5161 GCTGTCTGAA CTGGGGCAAT TCCCTGCAGA TCCTGGATCA GGTGGAGCTG AGAGCCGGCT
5221 ATCCACCCGC CATCCCACAC AACCTGTCTT GCCTGATGAA TCTGACAACC TCCTCTCTGA
5281 TCTGTCAGTG GGAGCCTGGA CCAGAGACCC ACCTGCCCAC AAGCTTCACC CTGAAGTCTT
5341 TTAAGAGCCG GGGCAACTGC CAGACACAGG GCGACAGCAT CCTGGATTGC GTGCCTAAGG
5401 ACGGCCAGTC CCACTGCTGT ATCCCAAGAA AGCACCTGCT GCTGTACCAG AACATGGGCA
5461 TCTGGGTGCA GGCCGAGAAT GCCCTGGGCA CCTCCATGTC TCCTCAGCTG TGCCTGGACC
5521 CAATGGATGT GGTGAAGCTG GAGCCTCCAA TGCTGCGGAC AATGGATCCA AGCCCTGAGG
5581 CAGCACCACC TCAGGCAGGA TGCCTGCAGC TGTGCTGGGA GCCTTGGCAG CCAGGCCTGC
5641 ACATCAATCA GAAGTGTGAG CTGAGGCACA AGCCTCAGAG GGGAGAGGCA TCCTGGGCAC
5701 TGGTGGGCCC ACTGCCCCTG GAGGCCCTGC AGTATGAGCT GTGCGGACTG CTGCCAGCAA
5761 CAGCATACAC CCTGCAGATC AGATGTATCA GGTGGCCTCT GCCAGGACAC TGGAGCGACT
5821 GGAGCCCATC CCTGGAGCTG AGAACAACCG AGAGGGCACC TACCGTGCGG CTGGACACAT
5881 GGTGGAGGCA GCGGCAGCTG GATCCAAGAA CCGTGCAGCT GTTCTGGAAG CCCGTGCCTC
5941 TGGAGGAGGA TTCCGGCAGG ATCCAGGGCT ATGTGGTGTC CTGGAGGCCA TCTGGACAGG
6001 CAGGAGCCAT CCTGCCTCTG TGCAACACAA CCGAGCTGAG CTGTACCTTC CACCTGCCAT
6061 CCGAGGCACA GGAGGTGGCC CTGGTGGCCT ACAATAGCGC CGGCACCTCC AGGCCAACAC
6121 CAGTGGTGTT TTCTGAGAGC CGCGGACCTG CCCTGACCAG GCTGCACGCA ATGGCAAGAG
6181 ACCCACACAG CCTGTGGGTG GGATGGGAGC CACCAAACCC TTGGCCACAG GGATACGTGA
6241 TCGAGTGGGG ACTGGGACCT CCATCCGCCT CTAACAGCAA TAAGACCTGG AGGATGGAGC
6301 AGAACGGCCG CGCCACAGGC TTCCTGCTGA AGGAGAATAT CCGCCCCTTT CAGCTGTATG
6361 AGATCATCGT GACCCCTCTG TACCAGGATA CAATGGGCCC AAGCCAGCAC GTGTATGCCT
6421 ACTCCCAGGA GATGGCCCCT TCTCACGCCC CAGAGCTGCA CCTGAAGCAC ATCGGCAAGA
6481 CCTGGGCACA GCTGGAGTGG GTGCCAGAGC CACCTGAGCT GGGCAAGTCC CCACTGACAC
6541 ACTATACCAT CTTCTGGACA AACGCCCAGA ATCAGTCCTT TTCTGCCATC CTGAACGCAA
6601 GCTCCCGGGG ATTCGTGCTG CACGGCCTGG AGCCCGCCTC CCTGTACCAC ATCCACCTGA
6661 TGGCAGCATC TCAGGCAGGA GCCACAAATA GCACCGTGCT GACACTGATG ACACTGACCC
6721 CTGAGGGCTC TGAGCTGCAC ATCATCCTGG GCCTGTTTGG CCTGCTGCTG CTGCTGACCT
6781 GCCTGTGCGG CACAGCCTGG CTGTGCTGTT CTCCCAACAG AAAGAATCCA CTGTGGCCTA
6841 GCGTGCCAGA CCCTGCACAC TCTAGCCTGG GAAGCTGGGT GCCAACCATC ATGGAGGAGG
6901 ATGCCTTTCA GCTGCCAGGA CTGGGAACAC CACCAATCAC CAAGCTGACA GTGCTGGAGG
6961 AGGACGAGAA GAAGCCAGTG CCCTGGGAGT CCCACAACTC CTCTTGA gtc gacaatcaac
7021 ctctggatta caaaatttgt gaaagattga ctggtattct taactatgtt gctcctttta
7081 cgctatgtgg atacgctgct ttaatgcctt tgtatcatgc tattgcttcc cgtatggctt
7141 tcattttctc ctccttgtat aaatcctggt tgctgtctct ttatgaggag ttgtggcccg
7201 ttgtcaggca acgtggcgtg gtgtgcactg tgtttgctga cgcaaccccc actggttggg
7261 gcattgccac cacctgtcag ctcctttccg ggactttcgc tttccccctc cctattgcca
7321 cggcggaact catcgccgcc tgccttgccc gctgctggac aggggctcgg ctgttgggca
7381 ctgacaattc cgtggtgttg tcggggaagc tgacgtcctt tccttggctg ctcgcctgtg
7441 ttgccacctg gattctgcgc gggacgtcct tctgctacgt cccttcggcc ctcaatccag
7501 cggaccttcc ttcccgcggc ctgctgccgg ctctgcggcc tcttccgcgt cttcgccttc
7561 gccctcagac gagtcggatc tccctttggg ccgcctcccc gcctggaatt cgagctcggt
7621 acctttaaga ccaatgactt acaaggcagc tgtagatctt agccactttt taaaagaaaa
7681 ggggggactg gaagggctaa ttcactccca acgaagacaa gatctgcttt ttgcttgtac
7741 tgggtctctc tggttagacc agatctgagc ctgggagctc tctggctaac tagggaaccc
7801 actgcttaag cctcaataaa gcttgccttg agtgcttcaa gtagtgtgtg cccgtctgtt
7861 gtgtgactct ggtaactaga gatccctcag acccttttag tcagtgtgga aaatctctag
7921 cagtagtagt tcatgtcatc ttattattca gtatttataa cttgcaaaga aatgaatatc
7981 agagagtgag aggaacttgt ttattgcagc ttataatggt tacaaataaa gcaatagcat
8041 cacaaatttc acaaataaag catttttttc actgcattct agttgtggtt tgtccaaact
8101 catcaatgta tcttatcatg tctggctcta gctatcccgc ccctaactcc gcccagttcc
8161 gcccattctc cgccccatgg ctgactaatt ttttttattt atgcagaggc cgaggccgcc
8221 tcggcctctg agctattcca gaagtagtga ggaggctttt ttggaggcct agctagggac
8281 gtacccaatt cgccctatag tgagtcgtat tacgcgcgct cactggccgt cgttttacaa
8341 cgtcgtgact gggaaaaccc tggcgttacc caacttaatc gccttgcagc acatccccct
8401 ttcgccagct ggcgtaatag cgaagaggcc cgcaccgatc gcccttccca acagttgcgc
8461 agcctgaatg gcgaatggga cgcgccctgt agcggcgcat taagcgcggc gggtgtggtg
8521 gttacgcgca gcgtgaccgc tacacttgcc agcgccctag cgcccgctcc tttcgctttc
8581 ttcccttcct ttctcgccac gttcgccggc tttccccgtc aagctctaaa tcgggggctc
8641 cctttagggt tccgatttag tgctttacgg cacctcgacc ccaaaaaact tgattagggt
8701 gatggttcac gtagtgggcc atcgccctga tagacggttt ttcgcccttt gacgttggag
8761 tccacgttct ttaatagtgg actcttgttc caaactggaa caacactcaa ccctatctcg
8821 gtctattctt ttgatttata agggattttg ccgatttcgg cctattggtt aaaaaatgag
8881 ctgatttaac aaaaatttaa cgcgaatttt aacaaaatat taacgcttac aatttaggtg
8941 gcacttttcg gggaaatgtg cgcggaaccc ctatttgttt atttttctaa atacattcaa
9001 atatgtatcc gctcatgaga caataaccct gataaatgct tcaataatat tgaaaaagga
9061 agagtatgag tattcaacat ttccgtgtcg cccttattcc cttttttgcg gcattttgcc
9121 ttcctgtttt tgctcaccca gaaacgctgg tgaaagtaaa agatgctgaa gatcagttgg
9181 gtgcacgagt gggttacatc gaactggatc tcaacaggg taagatcctt gagagttttc
9241 gccccgaaga acgttttcca atgatgagca cttttaaagt tctgctatgt ggcgcggtat
9301 tatcccgtat tgacgccggg caagagcaac tcggtcgccg catacactat tctcagaatg
9361 acttggttga gtactcacca gtcacagaaa agcatcttac ggatggcatg acagtaagag
9421 aattatgcag tgctgccata accatgagtg ataacactgc ggccaactta cttctgacaa
9481 cgatcggagg accgaaggag ctaaccgctt ttttgcacaa catgggggat catgtaactc
9541 gccttgatcg ttgggaaccg gagctgaatg aagccatacc aaacgacgag cgtgacacca
9601 cgatgcctgt agcaatggca acaacgttgc gcaaactatt aactggcgaa ctacttactc
9661 tagcttcccg gcaacaatta atagactgga tggaggcgga taaagttgca ggaccacttc
9721 tgcgctcggc ccttccggct ggctggttta ttgctgataa atctggagcc ggtgagcgtg
9781 ggtctcgcgg tatcattgca gcactggggc cagatggtaa gccctcccgt atcgtagtta
9841 tctacacgac ggggagtcag gcaactatgg atgaacgaaa tagacagatc gctgagatag
9901 gtgcctcact gattaagcat tggtaactgt cagaccaagt ttactcatat atactttaga
9961 ttgatttaaa acttcatttt taatttaaaa ggatctaggt gaagatcctt tttgataatc
10021 tcatgaccaa aatcccttaa cgtgagtttt cgttccactg agcgtcagac cccgtagaaa
10081 agatcaaagg atcttcttga gatccttttt ttctgcgcgt aatctgctgc ttgcaaacaa
10141 aaaaaccacc gctaccagcg gtggtttgtt tgccggatca agagctacca actctttttc
10201 cgaaggtaac tggcttcagc agagcgcaga taccaaatac tgttcttcta gtgtagccgt
10261 agttaggcca ccacttcaag aactctgtag caccgcctac atacctcgct ctgctaatcc
10321 tgttaccagt ggctgctgcc agtggcgata agtcgtgtct taccgggttg gactcaagac
10381 gatagttacc ggataaggcg cagcggtcgg gctgaacggg gggttcgtgc acacagccca
10441 gcttggagcg aacgacctac accgaactga gatacctaca ggtgagcta tgagaaagcg
10501 ccacgcttcc cgaagggaga aaggcggaca ggtatccggt aagcggcagg gtcggaacag
10561 gagagcgcac gagggagctt ccagggggaa acgcctggta tctttatagt cctgtcgggt
10621 ttcgccacct ctgacttgag cgtcgatttt tgtgatgctc gtcagggggg cggagcctat
10681 ggaaaaacgc cagcaacgcg gcctttttac ggttcctggc cttttgctgg ccttttgctc
10741 acatgttctt tcctgcgtta tcccctgatt ctgtggataa ccgtattacc gcctttgagt
10801 gagctgatac cgctcgccgc agccgaacga ccgagcgcag cgagtcagtg agcgaggaag
10861 cggaagagcg cccaatacgc aaaccgcctc tccccgcgcg ttggccgatt cattaatgca
10921 gctggcacga caggtttccc gactggaaag cgggcagtga gcgcaacgca attaatgtga
10981 gttagctcac tcattaggca ccccaggctt tacactttat gcttccggct cgtatgttgt
11041 gtggaattgt gagcggataa caatttcaca caggaaacag ctatgaccat gattacgcca
11101 agcgcgcaat taaccctcac taaagggaac aaaagctgga gctgcaagct taatgtagtc
11161 ttatgcaata ctcttgtagt cttgcaacat ggtaacgatg agttagcaac atgccttaca
11221 aggagagaaa aagcaccgtg catgccgatt ggtggaagta aggtggtacg atcgtgcctt
11281 attaggaagg caacagacgg gtctgacatg gattggacga accactgaat tgccgcattg
11341 cagagatatt gtatttaagt gcctagctcg atacataaac
Nucleotides 1 . . . 181 of SEQ ID NO: 5 = 5′ LTR (truncated);
228 . . . 353 = HIV-1 Psi;
846 . . . 1079-RRE;
1264 . . . 1308-gp41 peptide;
1962 . . . 2079-cPPT/CTS;
2133 . . . 3311 = EF-1-alpha promoter;
2364 . . . 3302 = EF-1-alpha intron A;
3324 . . . 7007 = car19-csf3r-d715;
7014 . . . 7602-WPRE;
complement(7485 . . . 7496) = Factor Xa site;
7689 . . . 7922-3′ LTR (Delta-U3);
7994 . . . 8115 = SV40 poly(A) signal;
8134 . . . 8269 = SV40 ori;
complement(8294 . . . 8312) = T7 promoter;
complement(8322 . . . 8338) = M13 fwd;
8480 . . . 8935 = f1 ori;
8961 . . . 9065 = AmpR promoter;
9066 . . . 9926 = AmpR;
10097 . . . 10685 = ori;
10973 . . . 10994 = CAP binding site;
11009 . . . 11039 = lac promoter;
11047 . . . 11063 = lac operator;
11071 . . . 11087 = M13 rev;
11108 . . . 11126 = T3 promoter;
11154 . . . 11380 = RSV promoter. CAR19-CSF3R-v1 vector (SEQ ID NO: 7);
(CAR19-CSF3R-v1 (SEQ ID NO: 8) comprises nucleotides 3324 . . . 7370)
      1 gggtctctct ggttagacca gatctgagcc tgggagctct ctggctaact agggaaccca
    61 ctgcttaagc ctcaataaag cttgccttga gtgcttcaag tagtgtgtgc ccgtctgttg
  121 tgtgactctg gtaactagag atccctcaga cccttttagt cagtgtggaa aatctctagc
  181 agtggcgccc gaacagggac ttgaaagcga aagggaaacc agaggagctc tctcgacgca
  241 ggactcggct tgctgaagcg cgcacggcaa gaggcgaggg gggcgactg gtgagtacgc
  301 caaaaatttt gactagcgga ggctagaagg agagagatgg gtgcgagagc gtcagtatta
  361 agcgggggag aattagatcg cgatgggaaa aaattcggtt aaggccaggg ggaaagaaaa
  421 aatataaatt aaaacatata gtatgggcaa gcagggagct agaacgattc gcagttaatc
  481 ctggcctgtt agaaacatca gaaggctgta gacaaatact gggacagcta caaccatccc
  541 ttcagacagg atcagaagaa cttagatcat tatataatac agtagcaacc ctctattgtg
  601 tgcatcaaag gatagagata aaagacacca aggaagcttt agacaagata gaggaagagc
  661 aaaacaaaag taagaccacc gcacagcaag cggccgctga tcttcagacc tggaggagga
  721 gatatgaggg acaattggag aagtgaatta tataaatata aagtagtaaa aattgaacca
  781 ttaggagtag cacccaccaa ggcaaagaga agagtggtgc agagagaaaa aagagcagtg
  841 ggaataggag ctttgttcct tgggttcttg ggagcagcag gaagcactat gggcgcagcg
  901 tcaatgacgc tgacggtaca ggccagacaa ttattgtctg gtatagtgca gcagcagaac
  961 aatttgctga gggctattga ggcgcaacag catctgttgc aactcacagt ctggggcatc
1021 aagcagctcc aggcaagaat cctggctgtg gaaagatacc taaaggatca acagctcctg
1081 gggatttggg gttgctctgg aaaactcatt tgcaccactg ctgtgccttg gaatgctagt
1141 tggagtaata aatctctgga acagatttgg aatcacacga cctggatgga gtgggacaga
1201 gaaattaaca attacacaag cttaatacac tccttaattg aagaatcgca aaaccagcaa
1261 gaaaagaatg aacaagaatt attggaatta gataaatggg caagtttgtg gaattggttt
1321 aacataacaa attggctgtg gtatataaaa ttattcataa tgatagtagg aggcttggta
1381 ggtttaagaa tagtttttgc tgtactttct atagtgaata gagttaggca gggatattca
1441 ccattatcgt ttcagaccca cctcccaacc ccgaggggac ccgacaggcc cgaaggaata
1501 gaagaagaag gtggagagag agacagagac agatccattc gattagtgaa cggatctcga
1561 cggtatcgat tagactgtag cccaggaata tggcagctag attgtacaca tttagaagga
1621 aaagttatct tggtagcagt tcatgtagcc agtggatata tagaagcaga agtaattcca
1681 gcagagacag ggcaagaaac agcatacttc ctcttaaaat tagcaggaag atggccagta
1741 aaaacagtac atacagacaa tggcagcaat ttcaccagta ctacagttaa ggccgcctgt
1801 tggtgggcgg ggatcaagca ggaatttggc attccctaca atccccaaag tcaaggagta
1861 atagaatcta tgaataaaga attaaagaaa attataggac aggtaagaga tcaggctgaa
1921 catcttaaga cagcagtaca aatggcagta ttcatccaca attttaaaag aaaagggggg
1981 attggggggt acagtgcagg ggaaagaata gtagacataa tagcaacaga catacaaact
2041 aaagaattac aaaaacaaat tacaaaaatt caaaattttc gggtttatta cagggacagc
2101 agagatccag tttggctgca tacgcgtcgt gaggctccgg tgcccgtcag tgggcagagc
2161 gcacatcgcc cacagtcccc gagaagttgg ggggaggggt cggcaattga accggtgcct
2221 agagaaggtg gcgcggggta aactgggaaa gtgatgtcgt gtactggctc cgcctttttc
2281 ccgagggtgg gggagaaccg tatataagtg cagtagtcgc cgtgaacgtt ctttttcgca
2341 acgggtttgc cgccagaaca caggtaagtg ccgtgtgtgg ttcccgcggg cctggcctct
2401 ttacgggtta tggcccttgc gtgccttgaa ttacttccac ctggctgcag tacgtgattc
2461 ttgatcccga gcttcgggtt ggaagtgggt gggagagttc gaggccttgc gcttaaggag
2521 ccccttcgcc tcgtgcttga gttgaggcct ggcctgggcg ctggggccgc cgcgtgcgaa
2581 tctggtggca ccttcgcgcc tgtctcgctg ctttcgataa gtctctagcc atttaaaatt
2641 tttgatgacc tgctgcgacg ctttttttct ggcaagatag tcttgtaaat gcgggccaag
2701 atctgcacac tggtatttcg gtttttgggg ccgcgggcgg cgacggggcc cgtgcgtccc
2761 agcgcacatg ttcggcgagg cggggcctgc gagcgcggcc accgagaatc ggacgggggt
2821 agtctcaagc tggccggcct gctctggtgc ctggcctcgc gccgccgtgt atcgccccgc
2881 cctgggcggc aaggctggcc cggtcggcac cagttgcgtg agcggaaaga tggccgcttc
2941 ccggccctgc tgcagggagc tcaaaatgga ggacgcggcg ctcgggagag cgggcgggtg
3001 agtcacccac acaaaggaaa agggcctttc cgtcctcagc cgtcgcttca tgtgactcca
3061 ctgagtaccg ggcgccgtcc aggcacctcg attagttctc gtgcttttgg agtacgtcgt
3121 ctttaggttg gggggagggg ttttatgcga tggagtttcc ccacactgag tgggtggaga
3181 ctgaagttag gccagcttgg cacttgatgt aattctcctt ggaatttgcc ctttttgagt
3241 ttggatcttg gttcattctc aagcctcaga cagtggttca aagttttttt cttccatttc
3301 aggtgtcgtg agctagctct aga ATGGCCC TGCCAGTGAC CGCCCTGCTG CTGCCACTGG
3361 CCCTGCTGCT GCACGCCGCC AGGCCCGACA TCCAGATGAC CCAGACCACA AGCTCCCTGA
3421 GCGCCTCCCT GGGCGACAGA GTGACAATCT CCTGCAGGGC CTCTCAGGAT ATCAGCAAGT
3481 ATCTGAACTG GTACCAGCAG AAGCCCGATG GCACCGTGAA GCTGCTGATC TATCACACAT
3541 CCCGGCTGCA CTCTGGCGTG CCTAGCAGAT TCTCTGGAAG CGGATCCGGA ACCGACTACT
3601 CCCTGACAAT CTCTAACCTG GAGCAGGAGG ATATCGCCAC CTATTTCTGC CAGCAGGGCA
3661 ATACCCTGCC TTACACATTT GGCGGCGGCA CCAAGCTGGA GATCACAGGC GGCGGCGGCT
3721 CTGGCGGAGG AGGAAGCGGA GGAGGAGGAT CCGAGGTGAA GCTGCAGGAG AGCGGACCAG
3781 GACTGGTGGC ACCTTCTCAG AGCCTGTCCG TGACATGTAC CGTGTCTGGC GTGAGCCTGC
3841 CAGACTACGG CGTGTCTTGG ATCAGGCAGC CACCTAGGAA GGGCCTGGAG TGGCTGGGCG
3901 TGATCTGGGG CAGCGAGACC ACATACTATA ATAGCGCCCT GAAGTCCCGC CTGACCATCA
3961 TCAAGGACAA CTCTAAGAGC CAGGTGTTCC TGAAGATGAA TAGCCTGCAG ACAGACGATA
4021 CCGCCATCTA CTATTGCGCC AAGCACTACT ATTACGGCGG CAGCTATGCC ATGGATTACT
4081 GGGGCCAGGG CACATCCGTG ACCGTGTCTA GCACCACAAC CCCTGCACCA AGGCCACCAA
4141 CCCCAGCACC TACAATCGCA TCTCAGCCAC TGAGCCTGCG CCCCGAGGCC TGTCGGCCTG
4201 CAGCAGGAGG AGCAGTGCAC ACCAGGGGCC TGGACTTTGC CTGCGATATC TACATCTGGG
4261 CACCACTGGC AGGAACCTGT GGCGTGCTGC TGCTGAGCCT GGTCATCACC CTGTATTGCA
4321 AGAGAGGCAG GAAGAAGCTG CTGTACATCT TCAAGCAGCC CTTCATGAGG CCCGTGCAGA
4381 CAACCCAGGA GGAGGACGGC TGCTCCTGTC GCTTCCCTGA GGAGGAGGAG GGCGGCTGTG
4441 AGCTGAGAGT GAAGTTTTCC AGGTCTGCCG ATGCCCCAGC CTATAAGCAG GGCCAGAACC
4501 AGCTGTACAA CGAGCTGAAT CTGGGCCGGA GAGAGGAGTA CGACGTGCTG GATAAGAGGA
4561 GGGGAAGGGA CCCAGAGATG GGAGGCAAGC CACGGAGAAA GAACCCCCAG GAGGGCCTGT
4621 ATAATGAGCT GCAGAAGGAC AAGATGGCCG AGGCCTACTC CGAGATCGGC ATGAAGGGAG
4681 AGAGGCGCCG GGGCAAGGGA CACGATGGCC TGTATCAGGG CCTGTCTACA GCCACCAAGG
4741 ACACCTACGA TGCCCTGCAC ATGCAGGCCC TGCCTCCAAG AACCAGCGGA TCCGGAGCAA
4801 CAAATTTCAG CCTGCTGAAG CAGGCAGGCG ACGTGGAGGA GAACCCAGGA CCACCTAGAA
4861 TGGCAAGGCT GGGCAATTGC AGCCTGACCT GGGCCGCCCT GATCATCCTG CTGCTGCCTG
4921 GCTCCCTGGA GGAGTGTGGC CACATCTCTG TGAGCGCCCC TATCGTGCAC CTGGGCGACC
4981 CAATCACAGC CTCCTGCATC ATCAAGCAGA ACTGTTCTCA CCTGGATCCA GAGCCACAGA
5041 TCCTGTGGAG GCTGGGAGCA GAGCTGCAGC CAGGAGGCCG CCAGCAGCGG CTGTCTGACG
5101 GCACCCAGGA GAGCATCATC ACACTGCCCC ACCTGAATCA CACCCAGGCC TTTCTGAGCT
5161 GCTGTCTGAA CTGGGGCAAT TCCCTGCAGA TCCTGGATCA GGTGGAGCTG AGGGCAGGAT
5221 ATCCACCAGC CATCCCACAC AACCTGAGCT GCCTGATGAA TCTGACAACC TCCTCTCTGA
5281 TCTGTCAGTG GGAGCCTGGA CCAGAGACCC ACCTGCCCAC ATCCTTCACC CTGAAGTCCT
5341 TTAAGTCTCG GGGCAACTGC CAGACACAGG GCGACTCTAT CCTGGATTGC GTGCCAAAGG
5401 ACGGCCAGAG CCACTGCTGT ATCCCCAGAA AGCACCTGCT GCTGTACCAG AACATGGGCA
5461 TCTGGGTGCA GGCCGAGAAT GCCCTGGGCA CCTCCATGTC TCCCCAGCTG TGCCTGGACC
5521 CTATGGATGT GGTGAAGCTG GAGCCTCCAA TGCTGCGGAC AATGGATCCA AGCCCTGAGG
5581 CAGCACCACC TCAGGCAGGA TGCCTGCAGC TGTGCTGGGA GCCTTGGCAG CCAGGCCTGC
5641 ACATCAATCA GAAGTGTGAG CTGAGGCACA AGCCTCAGAG GGGAGAGGCA TCCTGGGCAC
5701 TGGTGGGCCC ACTGCCCCTG GAGGCCCTGC AGTATGAGCT GTGCGGACTG CTGCCAGCAA
5761 CAGCATACAC CCTGCAGATC AGATGTATCA GGTGGCCTCT GCCAGGCCAC TGGAGCGACT
5821 GGAGCCCTTC CCTGGAGCTG AGAACAACCG AGAGGGCACC AACCGTGCGG CTGGACACAT
5881 GGTGGAGGCA GCGGCAGCTG GATCCAAGAA CCGTGCAGCT GTTCTGGAAG CCCGTGCCTC
5941 TGGAGGAGGA TTCCGGCAGG ATCCAGGGCT ATGTGGTGTC CTGGAGGCCT TCTGGACAGG
6001 CAGGAGCAAT CCTGCCACTG TGCAACACAA CCGAGCTGAG CTGTACCTTC CACCTGCCAT
6061 CCGAGGCACA GGAGGTGGCC CTGGTGGCAT ACAATAGCGC CGGCACCTCC CGGCCAACAC
6121 CAGTGGTGTT TTCTGAGAGC CGCGGACCAG CCCTGACCAG GCTGCACGCA ATGGCAAGAG
6181 ACCCACACAG CCTGTGGGTG GGATGGGAGC CACCAAACCC TTGGCCACAG GGATACGTGA
6241 TCGAGTGGGG ACTGGGACCT CCATCCGCCT CTAACAGCAA TAAGACCTGG AGGATGGAGC
6301 AGAACGGCCG CGCCACAGGC TTCCTGCTGA AGGAGAATAT CAGACCCTTT CAGCTGTATG
6361 AGATCATCGT GACCCCACTG TACCAGGATA CAATGGGCCC CAGCCAGCAC GTGTATGCCT
6421 ACTCCCAGGA GATGGCCCCT TCTCACGCCC CAGAGCTGCA CCTGAAGCAC ATCGGCAAGA
6481 CCTGGGCACA GCTGGAGTGG GTGCCAGAGC CACCTGAGCT GGGCAAGTCC CCTCTGACAC
6541 ACTATACCAT CTTCTGGACA AACGCCCAGA ATCAGTCCTT TTCTGCCATC CTGAACGCAA
6601 GCTCCAGGGG ATTCGTGCTG CACGGCCTGG AGCCAGCATC TCTGTACCAC ATCCACCTGA
6661 TGGCAGCATC TCAGGCAGGA GCCACAAATA GCACCGTGCT GACACTGATG ACACTGACCC
6721 CTGAGGGCTC CGAGCTGCAC ATCATCCTGG GCCTGTTTGG CCTGCTGCTG CTGCTGACCT
6781 GCCTGTGCGG CACAGCCTGG CTGTGCTGTT CTCCTAACCG CAAGAATCCA CTGTGGCCTA
6841 GCGTGCCAGA CCCTGCACAC TCTAGCCTGG GAAGCTGGGT GCCAACCATC ATGGAGGAGG
6901 ATGCCTTTCA GCTGCCTGGC CTGGGCACAC CACCCATCAC CAAGCTGACA GTGCTGGAGG
6961 AGGACGAGAA GAAGCCAGTG CCCTGGGAGT CCCACAACTC CTCTGAGACC TGCGGACTGC
7021 CCACCCTGGT GCAGACATAT GTGCTGCAGG GCGACCCAAG GGCCGTGTCC ACCCAGCCTC
7081 AGAGCCAGTC CGGCACATCT GATCAGGTGC TGTATGGCCA GCTGCTGGGA AGCCCTACCT
7141 CCCCTGGACC AGGACACTAC CTGAGATGCG ATAGCACCCA GCCACTGCTG GCAGGACTGA
7201 CACCATCTCC TAAGAGCTAC GAGAACCTGT GGTTCCAGGC ATCCCCACTG GGCACCCTGG
7261 TGACACCAGC CCCCTCTCAG GAGGACGATT GCGTGTTCGG CCCTCTGCTG AATTTTCCAC
7321 TGCTGCAGGG CATCAGAGTG CACGGCATGG AGGCCCTGGG CTCCTTTTGA gtcgacaatc
7381 aacctctgga ttacaaaatt tgtgaaagat tgactggtat tcttaactat gttgctcctt
7441 ttacgctatg tggatacgct gctttaatgc ctttgtatca tgctattgct tcccgtatgg
7501 ctttcatttt ctcctccttg tataaatcct ggttgctgtc tctttatgag gagttgtggc
7561 ccgttgtcag gcaacgtggc gtggtgtgca ctgtgtttgc tgacgcaacc cccactggtt
7621 ggggcattgc caccacctgt cagctccttt ccgggacttt cgctttcccc ctccctattg
7681 ccacggcgga actcatcgcc gcctgccttg cccgctgctg gacaggggct cggctgttgg
7741 gcactgacaa ttccgtggtg ttgtcgggga agctgacgtc ctttccttgg ctgctcgcct
7801 gtgttgccac ctggattctg cgcgggacgt ccttctgcta cgtcccttcg gccctcaatc
7861 cagcggacct tccttcccgc ggcctgctgc cggctctgcg gcctcttccg cgtcttcgcc
7921 ttcgccctca gacgagtcgg atctcccttt gggccgcctc cccgcctgga attcgagctc
7981 ggtaccttta agaccaatga cttacaaggc agctgtagat cttagccact ttttaaaaga
8041 aaagggggga ctggaagggc taattcactc ccaacgaaga caagatctgc tttttgcttg
8101 tactgggtct ctctggttag accagatctg agcctgggag ctctctggct aactagggaa
8161 cccactgctt aagcctcaat aaagcttgcc ttgagtgctt caagtagtgt gtgcccgtct
8221 gttgtgtgac tctggtaact agagatccct cagacccttt tagtcagtgt ggaaaatctc
8281 tagcagtagt agttcatgtc atcttattat tcagtattta taacttgcaa agaaatgaat
8341 atcagagagt gagaggaact tgtttattgc agcttataat ggttacaaat aaagcaatag
8401 catcacaaat ttcacaaata aagcattttt ttcactgcat tctagttgtg gtttgtccaa
8461 actcatcaat gtatcttatc atgtctggct ctagctatcc cgcccctaac tccgcccagt
8521 tccgcccatt ctccgcccca tggctgacta atttttttta tttatgcaga ggccgaggcc
8581 gcctcggcct ctgagctatt ccagaagtag tgaggaggct tttttggagg cctagctagg
8641 gacgtaccca attcgcccta tagtgagtcg tattacgcgc gctcactggc cgtcgtttta
8701 caacgtcgtg actgggaaaa ccctggcgtt acccaactta atcgccttgc agcacatccc
8761 cctttcgcca gctggcgtaa tagcgaagag gcccgcaccg atcgcccttc ccaacagttg
8821 cgcagcctga atggcgaatg ggacgcgccc tgtagcggcg cattaagcgc ggcgggtgtg
8881 gtggttacgc gcagcgtgac cgctacactt gccagcgccc tagcgcccgc tcctttcgct
8941 ttcttccctt cctttctcgc cacgttcgcc ggctttcccc gtcaagctct aaatcggggg
9001 ctccctttag ggttccgatt tagtgcttta cggcacctcg accccaaaaa acttgattag
9061 ggtgatggtt cacgtagtgg gccatcgccc tgatagacgg tttttcgccc tttgacgttg
9121 gagtccacgt tctttaatag tggactcttg ttccaaactg gaacaacact caaccctatc
9181 tcggtctatt cttttgattt ataagggatt ttgccgattt cggcctattg gttaaaaaat
9241 gagctgattt aacaaaaatt taacgcgaat tttaacaaaa tattaacgct tacaatttag
9301 gtggcacttt tcggggaaat gtgcgcggaa cccctatttg tttatttttc taaatacatt
9361 caaatatgta tccgctcatg agacaataac cctgataaat gcttcaataa tattgaaaaa
9421 ggaagagtat gagtattcaa catttccgtg tcgcccttat tccctttttt gcggcatttt
9481 gccttcctgt ttttgctcac ccagaaacgc tggtgaaagt aaaagatgct gaagatcagt
9541 tgggtgcacg agtgggttac atcgaactgg atctcaacag cggtaagatc cttgagagtt
9601 ttcgccccga agaacgtttt ccaatgatga gcacttttaa agttctgcta tgtggcgcgg
9661 tattatcccg tattgacgcc gggcaagagc aactcggtcg ccgcatacac tattctcaga
9721 atgacttggt tgagtactca ccagtcacag aaaagcatct tacggatggc atgacagtaa
9781 gagaattatg cagtgctgcc ataaccatga gtgataacac tgcggccaac ttacttctga
9841 caacgatcgg aggaccgaag gagctaaccg cttttttgca caacatgggg gatcatgtaa
9901 ctcgccttga tcgttgggaa ccggagctga atgaagccat accaaacgac gagcgtgaca
9961 ccacgatgcc tgtagcaatg gcaacaacgt tgcgcaaact attaactggc gaactactta
10021 ctctagcttc ccggcaacaa ttaatagact ggatggaggc ggataaagtt gcaggaccac
10081 ttctgcgctc ggcccttccg gctggctggt ttattgctga taaatctgga gccggtgagc
10141 gtgggtctcg cggtatcatt gcagcactgg ggccagatgg taagccctcc cgtatcgtag
10201 ttatctacac gacggggagt caggcaacta tggatgaacg aaatagacag atcgctgaga
10261 taggtgcctc actgattaag cattggtaac tgtcagacca agtttactca tatatacttt
10321 agattgattt aaaacttcat ttttaattta aaaggatcta ggtgaagatc ctttttgata
10381 atctcatgac caaaatccct taacgtgagt tttcgttcca ctgagcgtca gaccccgtag
10441 aaaagatcaa aggatcttct tgagatcctt tttttctgcg cgtaatctgc tgcttgcaaa
10501 caaaaaaacc accgctacca gcggtggttt gtttgccgga tcaagagcta ccaactcttt
10561 ttccgaaggt aactggcttc agcagagcgc agataccaaa tactgttctt ctagtgtagc
10621 cgtagttagg ccaccacttc aagaactctg tagcaccgcc tacatacctc gctctgctaa
10681 tcctgttacc agtggctgct gccagtggg ataagtcgtg tcttaccggg ttggactcaa
10741 gacgatagtt accggataag gcgcagcggt cgggctgaac ggggggttcg tgcacacagc
10801 ccagcttgga gcgaacgacc tacaccgaac tgagatacct acagcgtgag ctatgagaaa
10861 gcgccacgct tcccgaaggg agaaaggcgg acaggtatcc ggtaagcggc agggtcggaa
10921 caggagagcg cacgagggag cttccagggg gaaacgcctg gtatctttat agtcctgtcg
10981 ggtttcgcca cctctgactt gagcgtcgat ttttgtgatg ctcgtcaggg gggcggagcc
11041 tatggaaaaa cgccagcaac gcggcctttt tacggttcct ggccttttgc tggccttttg
11101 ctcacatgtt ctttcctgcg ttatcccctg attctgtgga taaccgtatt accgcctttg
11161 agtgagctga taccgctcgc cgcagccgaa cgaccgagcg cagcgagtca gtgagcgagg
11221 aagcggaaga gcgcccaata cgcaaaccgc ctctccccgc gcgttggccg attcattaat
11281 gcagctggca cgacaggttt cccgactgga aagcgggcag tgagcgcaac gcaattaatg
11341 tgagttagct cactcattag gcaccccagg ctttacactt tatgcttccg gctcgtatgt
11401 tgtgtggaat tgtgagcgga taacaatttc acacaggaaa cagctatgac catgattacg
11461 ccaagcgcgc aattaaccct cactaaaggg aacaaaagct ggagctgcaa gcttaatgta
11521 gtcttatgca atactcttgt agtcttgcaa catggtaacg atgagttagc aacatgcctt
11581 acaaggagag aaaaagcacc gtgcatgccg attggtggaa gtaaggtggt acgatcgtgc
11641 cttattagga aggcaacaga cgggtctgac atggattgga cgaaccactg aattgccgca
11701 ttgcagagat attgtattta agtgcctagc tcgatacata aac
CAR19-CSF3R-v4 vector (SEQ ID NO: 9);
(CAR19-CSF3R-v4 (SEQ ID NO: 10) comprises
nucleotides 3324 . . . 7211)
      1 gggtctctct ggttagacca gatctgagcc tgggagctct ctggctaact agggaaccca
    61 ctgcttaagc ctcaataaag cttgccttga gtgcttcaag tagtgtgtgc ccgtctgttg
  121 tgtgactctg gtaactagag atccctcaga cccttttagt cagtgtggaa aatctctagc
  181 agtggcgccc gaacagggac ttgaaagcga aagggaaacc agaggagctc tctcgacgca
  241 ggactcggct tgctgaagcg cgcacggcaa gaggcgaggg gggcgactg gtgagtacgc
  301 caaaaatttt gactagcgga ggctagaagg agagagatgg gtgcgagagc gtcagtatta
  361 agcgggggag aattagatcg cgatgggaaa aaattcggtt aaggccaggg ggaaagaaaa
  421 aatataaatt aaaacatata gtatgggcaa gcagggagct agaacgattc gcagttaatc
  481 ctggcctgtt agaaacatca gaaggctgta gacaaatact gggacagcta caaccatccc
  541 ttcagacagg atcagaagaa cttagatcat tatataatac agtagcaacc ctctattgtg
  601 tgcatcaaag gatagagata aaagacacca aggaagcttt agacaagata gaggaagagc
  661 aaaacaaaag taagaccacc gcacagcaag cggccgctga tcttcagacc tggaggagga
  721 gatatgaggg acaattggag aagtgaatta tataaatata aagtagtaaa aattgaacca
  781 ttaggagtag cacccaccaa ggcaaagaga agagtggtgc agagagaaaa aagagcagtg
  841 ggaataggag ctttgttcct tgggttcttg ggagcagcag gaagcactat gggcgcagcg
  901 tcaatgacgc tgacggtaca ggccagacaa ttattgtctg gtatagtgca gcagcagaac
  961 aatttgctga gggctattga ggcgcaacag catctgttgc aactcacagt ctggggcatc
1021 aagcagctcc aggcaagaat cctggctgtg gaaagatacc taaaggatca acagctcctg
1081 gggatttggg gttgctctgg aaaactcatt tgcaccactg ctgtgccttg gaatgctagt
1141 tggagtaata aatctctgga acagatttgg aatcacacga cctggatgga gtgggacaga
1201 gaaattaaca attacacaag cttaatacac tccttaattg aagaatcgca aaaccagcaa
1261 gaaaagaatg aacaagaatt attggaatta gataaatggg caagtttgtg gaattggttt
1321 aacataacaa attggctgtg gtatataaaa ttattcataa tgatagtagg aggcttggta
1381 ggtttaagaa tagtttttgc tgtactttct atagtgaata gagttaggca gggatattca
1441 ccattatcgt ttcagaccca cctcccaacc ccgaggggac ccgacaggcc cgaaggaata
1501 gaagaagaag gtggagagag agacagagac agatccattc gattagtgaa cggatctcga
1561 cggtatcgat tagactgtag cccaggaata tggcagctag attgtacaca tttagaagga
1621 aaagttatct tggtagcagt tcatgtagcc agtggatata tagaagcaga agtaattcca
1681 gcagagacag ggcaagaaac agcatacttc ctcttaaaat tagcaggaag atggccagta
1741 aaaacagtac atacagacaa tggcagcaat ttcaccagta ctacagttaa ggccgcctgt
1801 tggtgggcgg ggatcaagca ggaatttggc attccctaca atccccaaag tcaaggagta
1861 atagaatcta tgaataaaga attaaagaaa attataggac aggtaagaga tcaggctgaa
1921 catcttaaga cagcagtaca aatggcagta ttcatccaca attttaaaag aaaagggggg
1981 attggggggt acagtgcagg ggaaagaata gtagacataa tagcaacaga catacaaact
2041 aaagaattac aaaaacaaat tacaaaaatt caaaattttc gggtttatta cagggacagc
2101 agagatccag tttggctgca tacgcgtcgt gaggctccgg tgcccgtcag tgggcagagc
2161 gcacatcgcc cacagtcccc gagaagttgg ggggaggggt cggcaattga accggtgcct
2221 agagaaggtg gcgcggggta aactgggaaa gtgatgtcgt gtactggctc cgcctttttc
2281 ccgagggtgg gggagaaccg tatataagtg cagtagtcgc cgtgaacgtt ctttttcgca
2341 acgggtttgc cgccagaaca caggtaagtg ccgtgtgtgg ttcccgcggg cctggcctct
2401 ttacgggtta tggcccttgc gtgccttgaa ttacttccac ctggctgcag tacgtgattc
2461 ttgatcccga gcttcgggtt ggaagtgggt gggagagttc gaggccttgc gcttaaggag
2521 ccccttcgcc tcgtgcttga gttgaggcct ggcctgggcg ctggggccgc cgcgtgcgaa
2581 tctggtggca ccttcgcgcc tgtctcgctg ctttcgataa gtctctagcc atttaaaatt
2641 tttgatgacc tgctgcgacg ctttttttct ggcaagatag tcttgtaaat gcgggccaag
2701 atctgcacac tggtatttcg gtttttgggg ccgcgggcgg cgacggggcc cgtgcgtccc
2761 agcgcacatg ttcggcgagg cggggcctgc gagcgcggcc accgagaatc ggacgggggt
2821 agtctcaagc tggccggcct gctctggtgc ctggcctcgc gccgccgtgt atcgccccgc
2881 cctgggcggc aaggctggcc cggtcggcac cagttgcgtg agcggaaaga tggccgcttc
2941 ccggccctgc tgcagggagc tcaaaatgga ggacgcggcg ctcgggagag cgggcgggtg
3001 agtcacccac acaaaggaaa agggcctttc cgtcctcagc cgtcgcttca tgtgactcca
3061 ctgagtaccg ggcgccgtcc aggcacctcg attagttctc gtgcttttgg agtacgtcgt
3121 ctttaggttg gggggagggg ttttatgcga tggagtttcc ccacactgag tgggtggaga
3181 ctgaagttag gccagcttgg cacttgatgt aattctcctt ggaatttgcc ctttttgagt
3241 ttggatcttg gttcattctc aagcctcaga cagtggttca aagttttttt cttccatttc
3301 aggtgtcgtg agctagctct aga Atggcac tgcctgtgac cgccctgctg ctgccactgg
3361 ccctgctgct gcacgcagca aggccagaca tccagatgac ccagaccaca agctccctga
3421 gcgcctccct gggcgacagg gtgacaatca gctgcagggc ctcccaggat atctctaagt
3481 atctgaactg gtaccagcag aagccagatg gcaccgtgaa gctgctgatc tatcacacaa
3541 gcaggctgca ctccggagtg ccatctcgct tctctggaag cggatccgga accgactaca
3601 gcctgacaat ctccaacctg gagcaggagg atatcgccac ctatttctgc cagcagggca
3661 ataccctgcc ttacacattt ggcggcggca ccaagctgga gatcacaggc ggaggaggat
3721 ccggaggagg aggctctggc ggcggcggca gcgaggtgaa gctgcaggag agcggacctg
3781 gactggtggc accatctcag agcctgtccg tgacatgtac cgtgtctggc gtgagcctgc
3841 ccgactacgg cgtgtcttgg atcagacagc cacctaggaa gggcctggag tggctgggcg
3901 tgatctgggg cagcgagacc acatactata actctgccct gaagagccgc ctgaccatca
3961 tcaaggacaa ctctaagagc caggtgttcc tgaagatgaa ttccctgcag acagacgata
4021 ccgccatcta ctattgcgcc aagcactact attacggcgg ctcttatgcc atggattact
4081 ggggccaggg cacaagcgtg accgtgtcta gcaccacaac cccagcacca aggccaccaa
4141 cccctgcacc aacaatcgca tcccagccac tgtctctgag acctgaggca tgtaggccag
4201 cagcaggagg agcagtgcac accagaggcc tggactttgc ctgcgatatc tacatctggg
4261 caccactggc aggaacctgt ggcgtgctgc tgctgagcct ggtcatcacc ctgtattgca
4321 agcgcggccg gaagaagctg ctgtacatct tcaagcagcc ctttatgcgg cctgtgcaga
4381 caacccagga ggaggacggc tgctcctgta gattcccaga ggaggaggag ggaggatgtg
4441 agctgcgcgt gaagttttcc cggtctgccg atgcccctgc ctataagcag ggccagaacc
4501 agctgtacaa cgagctgaat ctgggccgga gagaggagta cgacgtgctg gataagagga
4561 ggggaaggga cccagagatg ggaggcaagc cccggagaaa gaaccctcag gagggcctgt
4621 ataatgagct gcagaaggac aagatggccg aggcctactc cgagatcggc atgaagggag
4681 agaggcgccg gggcaaggga cacgatggcc tgtatcaggg cctgtctaca gccaccaagg
4741 acacctacga tgccctgcac atgcaggccc tgcctccaag aaccagcgga tccggagcaa
4801 caaatttcag cctgctgaag caggcaggcg acgtggagga gaacccagga ccacctagga
4861 tggcaaggct gggcaattgc agcctgacct gggccgccct gatcatcctg ctgctgccag
4921 gatccctgga ggagtgtggc cacatctctg tgagcgcccc aatcgtgcac ctgggcgacc
4981 ctatcacagc cagctgcatc atcaagcaga actgttccca cctggatccc gagcctcaga
5041 tcctgtggag gctgggagca gagctgcagc ctggaggcag acagcagagg ctgtccgacg
5101 gaacccagga gtctatcatc acactgccac acctgaatca cacccaggcc tttctgtctt
5161 gctgtctgaa ctggggcaat agcctgcaga tcctggatca ggtggagctg agggcaggat
5221 atccaccagc catccctcac aacctgagct gcctgatgaa tctgacaacc tcctctctga
5281 tctgtcagtg ggagccagga ccagagaccc acctgccaac atccttcacc ctgaagtcct
5341 ttaagtctag gggcaactgc cagacacagg gcgactccat cctggattgc gtgcctaagg
5401 acggccagtc tcactgctgt atcccacgca agcacctgct gctgtaccag aacatgggca
5461 tctgggtgca ggccgagaat gccctgggca cctccatgtc tccacagctg tgcctggacc
5521 ccatggatgt ggtgaagctg gagcctccaa tgctgcggac aatggatcct agcccagagg
5581 cagcaccacc tcaggcagga tgcctgcagc tgtgctggga gccatggcag ccaggcctgc
5641 acatcaatca gaagtgtgag ctgagacaca agccccagag gggagaggca tcctgggcac
5701 tggtgggacc cctgcctctg gaggccctgc agtatgagct gtgcggactg ctgcctgcaa
5761 cagcatacac cctgcagatc aggtgtatca ggtggccact gccaggacac tggagcgact
5821 ggagcccatc cctggagctg aggacaaccg agagggcacc aaccgtgagg ctggacacat
5881 ggtggagaca gaggcagctg gatcctcgca ccgtgcagct gttctggaag cctgtgccac
5941 tggaggagga ttccggccgg atccagggct atgtggtgag ctggagacca tccggacagg
6001 caggagccat cctgcctctg tgcaacacaa ccgagctgtc ttgtaccttc cacctgccaa
6061 gcgaggcaca ggaggtggcc ctggtggcat acaattctgc cggcaccagc cggcccacac
6121 ctgtggtgtt ttctgagagc agaggacccg ccctgaccag gctgcacgca atggcaaggg
6181 accctcacag cctgtgggtg ggatgggagc caccaaaccc atggccacag ggatacgtga
6241 tcgagtgggg actgggacct ccatccgcct ctaacagcaa taagacctgg cggatggagc
6301 agaacggcag agccacaggc ttcctgctga aggagaatat cagacccttt cagctgtatg
6361 agatcatcgt gacccctctg taccaggata caatgggccc aagccagcac gtgtatgcct
6421 acagccagga gatggcacca tcccacgcac cagagctgca cctgaagcac atcggcaaga
6481 cctgggcaca gctggagtgg gtgccagagc cacctgagct gggcaagtcc ccactgacac
6541 actataccat cttctggaca aacgcccaga atcagtcctt ttctgccatc ctgaacgcaa
6601 gctccagggg attcgtgctg cacggcctgg agccagcatc cctgtaccac atccacctga
6661 tggcagcatc ccaggcagga gcaacaaatt ctaccgtgct gacactgatg acactgaccc
6721 ccgagggctc tgagctgcac atcatcctgg gcctgtttgg cctgctgctg ctgctgacct
6781 gcctgtgcgg cacagcctgg ctgtgctgta gccctaaccg caagaatcct ctgtggccat
6841 ccgtgcctga cccagcccac tctagcctgg gcagctgggt gccaaccatc atggaggagg
6901 atgcctttca gctgccagga ctgggaacac caccaatcac caagctgaca gtgctggagg
6961 aggacgagaa gaagcccgtg ccttgggagt ctcacaactc ctctgagacc tgcggactgc
7021 ccaccctggt gcagacatat gtgctgcagg gcgatccaag agccgtgagc acccagccac
7081 agagccagtc cggcacatcc gaccaggcag gacctccaag aaggtctgcc tacttcaagg
7141 atcagatcat gctgcaccct gcccccccta atggcctgct gtgcctgttt ccaatcacct
7201 ccgtgctgtg A gtcgacaat caacctctgg attacaaaat ttgtgaaaga ttgactggta
7261 ttcttaacta tgttgctcct tttacgctat gtggatacgc tgctttaatg cctttgtatc
7321 atgctattgc ttcccgtatg gctttcattt tctcctcctt gtataaatcc tggttgctgt
7381 ctctttatga ggagttgtgg cccgttgtca ggcaacgtgg cgtggtgtgc actgtgtttg
7441 ctgacgcaac ccccactggt tggggcattg ccaccacctg tcagctcctt tccgggactt
7501 tcgctttccc cctccctatt gccacggcgg aactcatcgc cgcctgcctt gcccgctgct
7561 ggacaggggc tcggctgttg ggcactgaca attccgtggt gttgtcgggg aagctgacgt
7621 cctttccttg gctgctcgcc tgtgttgcca cctggattct gcgcgggacg tccttctgct
7681 acgtcccttc ggccctcaat ccagcggacc ttccttcccg cggcctgctg ccggctctgc
7741 ggcctcttcc gcgtcttcgc cttcgccctc agacgagtcg gatctccctt tgggccgcct
7801 ccccgcctgg aattcgagct cggtaccttt aagaccaatg acttacaagg cagctgtaga
7861 tcttagccac tttttaaaag aaaagggggg actggaaggg ctaattcact cccaacgaag
7921 acaagatctg ctttttgctt gtactgggtc tctctggtta gaccagatct gagcctggga
7981 gctctctggc taactaggga acccactgct taagcctcaa taaagcttgc cttgagtgct
8041 tcaagtagtg tgtgcccgtc tgttgtgtga ctctggtaac tagagatccc tcagaccctt
8101 ttagtcagtg tggaaaatct ctagcagtag tagttcatgt catcttatta ttcagtattt
8161 ataacttgca aagaaatgaa tatcagagag tgagaggaac ttgtttattg cagcttataa
8221 tggttacaaa taaagcaata gcatcacaaa tttcacaaat aaagcatttt tttcactgca
8281 ttctagttgt ggtttgtcca aactcatcaa tgtatcttat catgtctggc tctagctatc
8341 ccgcccctaa ctccgcccag ttccgcccat tctccgcccc atggctgact aatttttttt
8401 atttatgcag aggccgaggc cgcctcggcc tctgagctat tccagaagta gtgaggaggc
8461 ttttttggag gcctagctag ggacgtaccc aattcgccct atagtgagtc gtattacgcg
8521 cgctcactgg ccgtcgtttt acaacgtcgt gactgggaaa accctggcgt tacccaactt
8581 aatcgccttg cagcacatcc ccctttcgcc agctggcgta atagcgaaga ggcccgcacc
8641 gatcgccctt cccaacagtt gcgcagcctg aatggcgaat gggacgcgcc ctgtagcggc
8701 gcattaagcg cggcgggtgt ggtggttacg cgcagcgtga ccgctacact tgccagcgcc
8761 ctagcgcccg ctcctttcgc tttcttccct tcctttctcg ccacgttcgc cggctttccc
8821 cgtcaagctc taaatcgggg gctcccttta gggttccgat ttagtgcttt acggcacctc
8881 gaccccaaaa aacttgatta gggtgatggt tcacgtagtg ggccatcgcc ctgatagacg
8941 gtttttcgcc ctttgacgtt ggagtccacg ttctttaata gtggactctt gttccaaact
9001 ggaacaacac tcaaccctat ctcggtctat tcttttgatt tataagggat tttgccgatt
9061 tcggcctatt ggttaaaaaa tgagctgatt taacaaaaat ttaacgcgaa ttttaacaaa
9121 atattaacgc ttacaattta ggtggcactt ttcggggaaa tgtgcgcgga acccctattt
9181 gtttattttt ctaaatacat tcaaatatgt atccgctcat gagacaataa ccctgataaa
9241 tgcttcaata atattgaaaa aggaagagta tgagtattca acatttccgt gtcgccctta
9301 ttcccttttt tgcggcattt tgccttcctg tttttgctca cccagaaacg ctggtgaaag
9361 taaaagatgc tgaagatcag ttgggtgcac gagtgggtta catcgaactg gatctcaaca
9421 gcggtaagat ccttgagagt tttcgccccg aagaacgttt tccaatgatg agcactttta
9481 aagttctgct atgtggcgcg gtattatccc gtattgacgc cgggcaagag caactcggtc
9541 gccgcataca ctattctcag aatgacttgg ttgagtactc accagtcaca gaaaagcatc
9601 ttacggatgg catgacagta agagaattat gcagtgctgc cataaccatg agtgataaca
9661 ctgcggccaa cttacttctg acaacgatcg gaggaccgaa ggagctaacc gcttttttgc
9721 acaacatggg ggatcatgta actcgccttg atcgttggga accggagctg aatgaagcca
9781 taccaaacga cgagcgtgac accacgatgc ctgtagcaat ggcaacaacg ttgcgcaaac
9841 tattaactgg cgaactactt actctagctt cccggcaaca attaatagac tggatggagg
9901 cggataaagt tgcaggacca cttctgcgct cggcccttcc ggctggctgg tttattgctg
9961 ataaatctgg agccggtgag cgtgggtctc gcggtatcat tgcagcactg gggccagatg
10021 gtaagccctc ccgtatcgta gttatctaca cgacggggag tcaggcaact atggatgaac
10081 gaaatagaca gatcgctgag ataggtgcct cactgattaa gcattggtaa ctgtcagacc
10141 aagtttactc atatatactt tagattgatt taaaacttca tttttaattt aaaaggatct
10201 aggtgaagat cctttttgat aatctcatga ccaaaatccc ttaacgtgag ttttcgttcc
10261 actgagcgtc agaccccgta gaaaagatca aaggatcttc ttgagatcct ttttttctgc
10321 gcgtaatctg ctgcttgcaa acaaaaaaac caccgctacc agcggtggtt tgtttgccgg
10381 atcaagagct accaactctt tttccgaagg taactggctt cagcagagcg cagataccaa
10441 atactgttct tctagtgtag ccgtagttag gccaccactt caagaactct gtagcaccgc
10501 ctacatacct cgctctgcta atcctgttac cagtggctgc tgccagtggc gataagtcgt
10561 gtcttaccgg gttggactca agacgatagt taccggataa ggcgcagcgg tcgggctgaa
10621 cggggggttc gtgcacacag cccagcttgg agcgaacgac ctacaccgaa ctgagatacc
10681 tacagcgtga gctatgagaa agcgccacgc ttcccgaagg gagaaaggcg gacaggtatc
10741 cggtaagcgg cagggtcgga acaggagagc gcacgaggga gcttccaggg ggaaacgcct
10801 ggtatcttta tagtcctgtc gggtttcgcc acctctgact tgagcgtcga tttttgtgat
10861 gctcgtcagg ggggcggagc ctatggaaaa acgccagcaa cgcggccttt ttacggttcc
10921 tggccttttg ctggcctttt gctcacatgt tctttcctgc gttatcccct gattctgtgg
10981 ataaccgtat taccgccttt gagtgagctg ataccgctcg ccgcagccga acgaccgagc
11041 gcagcgagtc agtgagcgag gaagcggaag agcgcccaat acgcaaaccg cctctccccg
11101 cgcgttggcc gattcattaa tgcagctggc acgacaggtt tcccgactgg aaagcgggca
11161 gtgagcgcaa cgcaattaat gtgagttagc tcactcatta ggcaccccag gctttacact
11221 ttatgcttcc ggctcgtatg ttgtgtggaa ttgtgagcgg ataacaattt cacacaggaa
11281 acagctatga ccatgattac gccaagcgcg caattaaccc tcactaaagg gaacaaaagc
11341 tggagctgca agcttaatgt agtcttatgc aatactcttg tagtcttgca acatggtaac
11401 gatgagttag caacatgcct tacaaggaga gaaaaagcac cgtgcatgcc gattggtgga
11461 agtaaggtgg tacgatcgtg ccttattagg aaggcaacag acgggtctga catggattgg
11521 acgaaccact gaattgccgc attgcagaga tattgtattt aagtgcctag ctcgatacat
11581 aaac
```

### ENUMERATED EMBODIMENTS

The following enumerated embodiments are provided, the numbering of which is not to be construed as designating levels of importance.

Embodiment 1 provides a method of controlling T cell expansion in a subject, the method comprising: administering to the subject, a modified T cell comprising an ectopic Granulocyte Colony Stimulating Factor receptor (G-CSFR); administering Granulocyte Colony Stimulating Factor (G-CSF) to the subject to stimulate expansion of the T cell; and/or administering an inhibitor of G-CSF, JAK 2, or JAK1/2 to the subject to inhibit expansion of the T cell.

Embodiment 2 provides the method of embodiment 1, wherein the G-CSFR is an alternatively spliced class IV G-CSFR.

Embodiment 3 provides the method of embodiment 1, wherein the G-CSF is recombinant human G-CSF (rhG-CSF).

Embodiment 4 provides the method of any preceding embodiment, wherein the T cell comprises a chimeric antigen receptor (CAR).

Embodiment 5 provides a modified immune cell or precursor cell thereof, comprising a chimeric antigen receptor (CAR) and an exogeneous/exogeneous G-CSF receptor (G-CSFR).

Embodiment 6 provides the modified immune cell or precursor cell thereof of embodiment 4, wherein the G-CSFR is an alternatively spliced class IV G-CSFR.

Embodiment 7 provides the modified immune cell or precursor cell thereof of embodiment 5, wherein the G-CSFR comprises a deletion at aa 715 (GCSFRd715).

Embodiment 8 provides the modified immune cell or precursor cell thereof of any preceding embodiment, wherein the immune cell or precursor cell thereof is a T cell.

Embodiment 9 provides the modified immune cell or precursor cell thereof of any preceding embodiment, wherein the T cell is a human T cell.

Embodiment 10 provides the modified immune cell or precursor cell thereof of any preceding embodiment, wherein the cell is an autologous cell.

Embodiment 11 provides a method of treating a disease or disorder in a subject in need thereof, the method comprising administering to the subject a modified T cell comprising a CAR and an ectopic G-CSFR.

Embodiment 12 provides the method of embodiment 11, wherein the disease or disorder is cancer.

Embodiment 13 provides the method of embodiment 11, further comprising administering G-CSF to the subject to stimulate expansion of the T cell.

Embodiment 14 provides the method of embodiment 11, further comprising administering an inhibitor of G-CSF, JAK 2, or JAK1/2 to the subject to inhibit expansion of the T cell.

Embodiment 15 provides the method of any of embodiments 11-14, wherein the method is used to prevent chemotherapy-associated neutropenia.

Embodiment 16 provides the method of embodiment 11, wherein the G-CSFR is an alternatively spliced class IV G-CSFR.

Embodiment 17 provides the method of embodiment 11, wherein the G-CSFR comprises a deletion at aa 715 (GCSFRd715).

Embodiment 18 provides the method of embodiment 11, wherein the G-CSF is recombinant rhG-CSF.

Embodiment 19 provides a method of generating a population of cells for use as an immunotherapy, the method comprising introducing into an immune cell or precursor cell thereof a measles virus (MV) pseudotyped lentiviral vector encoding a membrane bound version of IL15 (mbIL15) and a chimeric antigen receptor (CAR).

Embodiment 20 provides the method of embodiment 19, wherein the immune cell or precursor cell thereof is a T cell.

Embodiment 21 provides the method of embodiment 20, wherein the T cell is human T cell.

Embodiment 22 provides the method of embodiment 19 or 20, wherein the T cell is an autologous T cell.

Embodiment 23 provides the method of embodiment 19, wherein the mbIL15 comprises a fusion protein comprising IL15 linked to IL15Rα via a flexible linker.

Embodiment 24 provides the method of embodiment 19, wherein the method generates a population of cells that is 1-, 2-, 5-, or 10-fold higher in cell number compared to a method that does not comprise a measles virus (MV) pseudotyped lentiviral vector encoding mbIL15.

Embodiment 25 provides a method of expanding CAR T cells, the method comprising introducing into the CAR T cell a measles virus (MV) pseudotyped lentiviral vector encoding a membrane bound version of IL15 (mbIL15).

Embodiment 26 provides the method of embodiment 25, wherein the mbIL15 comprises a fusion protein comprising IL15 linked to IL15Rα via a flexible linker.

Embodiment 27 provides the method of embodiment 25, wherein the method generates a population of cells that is 1-, 2-, 5-, or 10-fold higher in cell number compared to a method that does not comprise a measles virus (MV) pseudotyped lentiviral vector encoding mbIL15.

Embodiment 28 provides a method of treating cancer in a subject in need thereof, the method comprising administering to the subject a population of cells generated by the method of claim 16 or the expanded CAR T cells of embodiment 25.

Embodiment 29 provides a composition comprising a measles virus (MV) pseudotyped lentiviral vector comprising a nucleotide sequence encoding membrane-bound IL15 and a nucleotide sequence encoding a chimeric antigen receptor (CAR).

Embodiment 30 provides the composition of embodiment 29, wherein the vector comprises a nucleotide sequence at least 85%, 90%, 95%, 95%, 96%, 97%, 98%, 99%, or 100% identical to SEQ ID NO: 1.

Embodiment 31 provides a composition comprising a measles virus (MV) pseudotyped lentiviral vector comprising a nucleotide sequence encoding CSF3R and a nucleotide sequence encoding a chimeric antigen receptor (CAR).

Embodiment 32 provides the composition of embodiment 31, wherein the vector comprises a nucleotide sequence at least 85%, 90%, 95%, 95%, 96%, 97%, 98%, 99%, or 100% identical to SEQ ID NOs: 2-4.

Embodiment 33 provides the modified immune cell or precursor cell of any preceding embodiment, wherein the CAR comprises an antigen binding domain, a transmembrane domain, and an intracellular domain.

Embodiment 34 provides the modified immune cell or precursor cell of embodiment 33,wherein the antigen binding domain is selected from the group consisting of an antibody, an scFv, and a Fab.

Embodiment 35 provides the modified immune cell or precursor cell of embodiment 33,wherein the antigen binding domain is capable of binding a tumor associated antigen (TAA).

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

What is claimed is:

1. A method of controlling T cell expansion in a subject, the method comprising
administering to the subject, a modified T cell comprising an ectopic Granulocyte Colony Stimulating Factor receptor (G-CSFR),
administering Granulocyte Colony Stimulating Factor (G-CSF) to the subject to stimulate expansion of the T cell, and/or
administering an inhibitor of G-CSF, JAK 2, or JAK1/2 to the subject to inhibit expansion of the T cell.

2. The method of claim 1, wherein the G-CSFR is an alternatively spliced class IV G-CSFR.

3. The method of claim 1, wherein the G-CSF is recombinant human G-CSF (rhG-CSF).

4. The method of claim 1, wherein the T cell comprises a chimeric antigen receptor (CAR).

5. A modified immune cell or precursor cell thereof, comprising a chimeric antigen receptor (CAR) and an ectopic/exogeneous G-CSF receptor (G-CSFR).

6. The modified immune cell or precursor cell thereof of claim 5, wherein the G-CSFR is an alternatively spliced class IV G-CSFR.

7. The modified immune cell or precursor cell thereof of claim 5, wherein the G-CSFR comprises a deletion at aa 715 (GCSFRd715).

8. The modified immune cell or precursor cell thereof of claim 5, wherein the immune cell or precursor cell thereof is a T cell.

9. The modified immune cell or precursor cell thereof of claim 8, wherein the T cell is a human T cell.

10. The modified immune cell or precursor cell thereof of claim 5, wherein the cell is an autologous cell.

11. A method of treating a disease or disorder in a subject in need thereof, the method comprising administering to the subject a modified T cell comprising a CAR and an ectopic G-CSFR.

12. The method of claim 11, wherein the disease or disorder is cancer.

13. The method of claim 11, further comprising administering G-CSF to the subject to stimulate expansion of the T cell.

14. The method of claim 11, further comprising administering an inhibitor of G-CSF, JAK 2, or JAK1/2 to the subject to inhibit expansion of the T cell.

15. The method of claim 11, wherein the method is used to prevent chemotherapy-associated neutropenia.

16. The method of claim 11, wherein the G-CSFR is an alternatively spliced class IV G-CSFR.

17. The method of claim 11, wherein the G-CSFR comprises a deletion at aa 715 (GCSFRd715).

18. The method of claim 11, wherein the G-CSF is recombinant rhG-CSF.

19. A method of generating a population of cells for use as an immunotherapy, the method comprising introducing into an immune cell or precursor cell thereof a measles virus (MV) pseudotyped lentiviral vector encoding a membrane bound version of IL15 (mbIL15) and a chimeric antigen receptor (CAR).

20. The method of claim 19, wherein the immune cell or precursor cell thereof is a T cell.

21. The method of claim 20, wherein the T cell is human T cell.

22. The method of claim 19, wherein the T cell is an autologous T cell.

23. The method of claim 19, wherein the mbIL15 comprises a fusion protein comprising IL15 linked to IL15Rα via a flexible linker.

24. The method of claim 19, wherein the method generates a population of cells that is 1-, 2-, 5-, or 10-fold higher in cell number compared to a method that does not comprise a measles virus (MV) pseudotyped lentiviral vector encoding mbIL15.

25. A method of expanding CAR T cells, the method comprising introducing into the CAR T cell a measles virus (MV) pseudotyped lentiviral vector encoding a membrane bound version of IL15 (mbIL15).

26. The method of claim 25, wherein the mbIL15 comprises a fusion protein comprising IL15 linked to IL15Rα via a flexible linker.

27. The method of claim 25, wherein the method generates a population of cells that is 1-, 2-, 5-, or 10-fold higher in cell number compared to a method that does not comprise a measles virus (MV) pseudotyped lentiviral vector encoding mbIL15.

28. A method of treating cancer in a subject in need thereof, the method comprising administering to the subject the expanded CAR T cells of claim 25.

29. A composition comprising a measles virus (MV) pseudotyped lentiviral vector comprising a nucleotide sequence encoding membrane-bound IL15 and a nucleotide sequence encoding a chimeric antigen receptor (CAR).

30. The composition of claim 29, wherein the vector comprises a nucleotide sequence at least 85%, 90%, 95%, 95%, 96%, 97%, 98%, 99%, or 100% identical to SEQ ID NO: 1.

31. A composition comprising a measles virus (MV) pseudotyped lentiviral vector comprising a nucleotide sequence encoding CSF3R and a nucleotide sequence encoding a chimeric antigen receptor (CAR).

32. The composition of claim 31, wherein the vector comprises a nucleotide sequence at least 85%, 90%, 95%, 95%, 96%, 97%, 98%, 99%, or 100% identical to SEQ ID NOs: 2-4.

33. The modified immune cell or precursor cell of claim 31, wherein the CAR comprises an antigen binding domain, a transmembrane domain, and an intracellular domain.

34. The modified immune cell or precursor cell of claim 33, wherein the antigen binding domain is selected from the group consisting of an antibody, an scFv, and a Fab.

35. The modified immune cell or precursor cell of claim 33, wherein the antigen binding domain is capable of binding a tumor associated antigen (TAA).


SEQUENCE LISTING (38 sequences)

SEQ ID NO: 1 - protein, 422 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLSRMDYKDDDDKDYKDDDDKDYKDDDDK

SEQ ID NO: 2 - DNA, 12091 bp, synthetic construct
  source 1..12091: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagctctagagccgccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcactcgacgaatctaagtacggaccgccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgcggagcaagcggagcagaggcggccacagcgactacatgaacatgacccccagacggcctggccccacccggaagcactaccagccctacgccccacccagggactttgccgcctacagaagcaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatggcaaggctgggaaactgcagcctgacttgggctgccctgatcatcctgctgctccccggaagtctggaggagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacggatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgtcccactccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcctcctcccgtggctttgtcctccatggcctggagcccgccagtctgtatcacatccacctcatggctgccagccaggctggggccaccaacagtacagtcctcaccctgatgaccttgaccccagaggggtcggagctacacatcatcctgggcctgttcggcctcctgctgttgctcacctgcctctgtggaactgcctggctctgttgcagccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcatagagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaac

SEQ ID NO: 3 - DNA, 12454 bp, synthetic construct
  source 1..12454: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagctctagagccgccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcactcgacgaatctaagtacggaccgccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgcggagcaagcggagcagaggcggccacagcgactacatgaacatgacccccagacggcctggccccacccggaagcactaccagccctacgccccacccagggactttgccgcctacagaagcaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatggcaaggctgggaaactgcagcctgacttgggctgccctgatcatcctgctgctccccggaagtctggaggagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacggatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgtcccactccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcctcctcccgtggctttgtcctccatggcctggagcccgccagtctgtatcacatccacctcatggctgccagccaggctggggccaccaacagtacagtcctcaccctgatgaccttgaccccagaggggtcggagctacacatcatcctgggcctgttcggcctcctgctgttgctcacctgcctctgtggaactgcctggctctgttgcagccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcagagacctgtggcctccccactctggtccagacctatgtgctccagggggacccaagagcagtttccacccagccccaatcccagtctggcaccagcgatcaggtcctttatgggcagctgctgggcagccccacaagcccagggccagggcactatctccgctgtgactccactcagcccctcttggcgggcctcacccccagccccaagtcctatgagaacctctggttccaggccagccccttggggaccctggtaaccccagccccaagccaggaggacgactgtgtctttgggccactgctcaacttccccctcctgcaggggatccgggtccatgggatggaggcgctggggagcttctagagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaac

SEQ ID NO: 4 - DNA, 12295 bp, synthetic construct
  source 1..12295: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagctctagagccgccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcactcgacgaatctaagtacggaccgccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgcggagcaagcggagcagaggcggccacagcgactacatgaacatgacccccagacggcctggccccacccggaagcactaccagccctacgccccacccagggactttgccgcctacagaagcaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatggcaaggctgggaaactgcagcctgacttgggctgccctgatcatcctgctgctccccggaagtctggaggagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacggatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgtcccactccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcctcctcccgtggctttgtcctccatggcctggagcccgccagtctgtatcacatccacctcatggctgccagccaggctggggccaccaacagtacagtcctcaccctgatgaccttgaccccagaggggtcggagctacacatcatcctgggcctgttcggcctcctgctgttgctcacctgcctctgtggaactgcctggctctgttgcagccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcagagacctgtggcctccccactctggtccagacctatgtgctccagggggacccaagagcagtttccacccagccccaatcccagtctggcaccagcgatcaggctgggcctcccaggcgatctgcatactttaaggaccagatcatgctccatccagccccacccaatggccttttgtgcttgtttcctataacttcagtattgtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaac

SEQ ID NO: 5 - DNA, 11380 bp, synthetic construct
  source 1..11380: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagctctagaatggcactgccagtgaccgccctgctgctgcctctggccctgctgctgcacgcagcaaggccagacatccagatgacccagaccacaagctccctgagcgcctccctgggcgacagagtgacaatctcctgcagggcctctcaggatatcagcaagtatctgaactggtaccagcagaagcccgatggcaccgtgaagctgctgatctatcacacatcccggctgcactctggcgtgcctagcagattctctggaagcggatccggaaccgactactccctgacaatctctaacctggagcaggaggatatcgccacctatttctgccagcagggcaataccctgccttacacatttggcggcggcaccaagctggagatcacaggcggcggcggctctggaggaggaggaagcggaggaggaggatccgaggtgaagctgcaggagagcggaccaggactggtggcaccttctcagagcctgtccgtgacatgcaccgtgagcggcgtgtccctgccagactacggcgtgtcctggatcaggcagccacctaggaagggcctggagtggctgggcgtgatctggggctctgagaccacatactataatagcgccctgaagtccagactgaccatcatcaaggacaactctaagagccaggtgttcctgaagatgaattccctgcagacagacgataccgccatctactattgcgccaagcactactattacggcggctcctatgccatggattactggggccagggcacatctgtgaccgtgtctagcaccacaacccctgcaccaaggccaccaaccccagcacctacaatcgcatctcagccactgagcctgcgccccgaggcctgtcggcctgcagcaggaggagcagtgcacaccaggggcctggactttgcctgcgatatctacatctgggcacctctggcaggaacctgtggcgtgctgctgctgagcctggtcatcaccctgtattgcaagagaggcaggaagaagctgctgtacatcttcaagcagcccttcatgaggcccgtgcagacaacccaggaggaggacggctgctcctgtcgcttccctgaagaggaggagggcggctgtgagctgagagtgaagttttccaggtctgccgatgccccagcctataagcagggccagaaccagctgtacaacgagctgaatctgggccggagagaggagtacgacgtgctggataagaggaggggaagggacccagagatgggaggcaagccacggagaaagaacccccaggagggcctgtataatgagctgcagaaggacaagatggccgaggcctacagcgagatcggcatgaagggagagaggcgccggggcaagggacacgatggcctgtatcagggcctgtccacagccaccaaggacacctacgatgcactgcacatgcaggccctgcctccacgcaccagcggatccggcgccacaaatttcagcctgctgaagcaggcaggcgatgtggaggagaacccaggaccacctagaatggcaaggctgggcaattgctctctgacctgggccgccctgatcatcctgctgctgcctggcagcctggaggagtgtggccacatctctgtgagcgccccaatcgtgcacctgggcgaccccatcacagcctcctgcatcatcaagcagaactgttctcacctggatccagagccccagatcctgtggaggctgggagcagagctgcagccaggaggccgccagcagcggctgtctgacggcacccaggagagcatcatcacactgccccacctgaatcacacccaggcctttctgagctgctgtctgaactggggcaattccctgcagatcctggatcaggtggagctgagagccggctatccacccgccatcccacacaacctgtcttgcctgatgaatctgacaacctcctctctgatctgtcagtgggagcctggaccagagacccacctgcccacaagcttcaccctgaagtcttttaagagccggggcaactgccagacacagggcgacagcatcctggattgcgtgcctaaggacggccagtcccactgctgtatcccaagaaagcacctgctgctgtaccagaacatgggcatctgggtgcaggccgagaatgccctgggcacctccatgtctcctcagctgtgcctggacccaatggatgtggtgaagctggagcctccaatgctgcggacaatggatccaagccctgaggcagcaccacctcaggcaggatgcctgcagctgtgctgggagccttggcagccaggcctgcacatcaatcagaagtgtgagctgaggcacaagcctcagaggggagaggcatcctgggcactggtgggcccactgcccctggaggccctgcagtatgagctgtgcggactgctgccagcaacagcatacaccctgcagatcagatgtatcaggtggcctctgccaggacactggagcgactggagcccatccctggagctgagaacaaccgagagggcacctaccgtgcggctggacacatggtggaggcagcggcagctggatccaagaaccgtgcagctgttctggaagcccgtgcctctggaggaggattccggcaggatccagggctatgtggtgtcctggaggccatctggacaggcaggagccatcctgcctctgtgcaacacaaccgagctgagctgtaccttccacctgccatccgaggcacaggaggtggccctggtggcctacaatagcgccggcacctccaggccaacaccagtggtgttttctgagagccgcggacctgccctgaccaggctgcacgcaatggcaagagacccacacagcctgtgggtgggatgggagccaccaaacccttggccacagggatacgtgatcgagtggggactgggacctccatccgcctctaacagcaataagacctggaggatggagcagaacggccgcgccacaggcttcctgctgaaggagaatatccgcccctttcagctgtatgagatcatcgtgacccctctgtaccaggatacaatgggcccaagccagcacgtgtatgcctactcccaggagatggccccttctcacgccccagagctgcacctgaagcacatcggcaagacctgggcacagctggagtgggtgccagagccacctgagctgggcaagtccccactgacacactataccatcttctggacaaacgcccagaatcagtccttttctgccatcctgaacgcaagctcccggggattcgtgctgcacggcctggagcccgcctccctgtaccacatccacctgatggcagcatctcaggcaggagccacaaatagcaccgtgctgacactgatgacactgacccctgagggctctgagctgcacatcatcctgggcctgtttggcctgctgctgctgctgacctgcctgtgcggcacagcctggctgtgctgttctcccaacagaaagaatccactgtggcctagcgtgccagaccctgcacactctagcctgggaagctgggtgccaaccatcatggaggaggatgcctttcagctgccaggactgggaacaccaccaatcaccaagctgacagtgctggaggaggacgagaagaagccagtgccctgggagtcccacaactcctcttgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaac

SEQ ID NO: 6 - DNA, 3684 bp, synthetic construct
  source 1..3684: mol_type = other DNA
atggcactgccagtgaccgccctgctgctgcctctggccctgctgctgcacgcagcaaggccagacatccagatgacccagaccacaagctccctgagcgcctccctgggcgacagagtgacaatctcctgcagggcctctcaggatatcagcaagtatctgaactggtaccagcagaagcccgatggcaccgtgaagctgctgatctatcacacatcccggctgcactctggcgtgcctagcagattctctggaagcggatccggaaccgactactccctgacaatctctaacctggagcaggaggatatcgccacctatttctgccagcagggcaataccctgccttacacatttggcggcggcaccaagctggagatcacaggcggcggcggctctggaggaggaggaagcggaggaggaggatccgaggtgaagctgcaggagagcggaccaggactggtggcaccttctcagagcctgtccgtgacatgcaccgtgagcggcgtgtccctgccagactacggcgtgtcctggatcaggcagccacctaggaagggcctggagtggctgggcgtgatctggggctctgagaccacatactataatagcgccctgaagtccagactgaccatcatcaaggacaactctaagagccaggtgttcctgaagatgaattccctgcagacagacgataccgccatctactattgcgccaagcactactattacggcggctcctatgccatggattactggggccagggcacatctgtgaccgtgtctagcaccacaacccctgcaccaaggccaccaaccccagcacctacaatcgcatctcagccactgagcctgcgccccgaggcctgtcggcctgcagcaggaggagcagtgcacaccaggggcctggactttgcctgcgatatctacatctgggcacctctggcaggaacctgtggcgtgctgctgctgagcctggtcatcaccctgtattgcaagagaggcaggaagaagctgctgtacatcttcaagcagcccttcatgaggcccgtgcagacaacccaggaggaggacggctgctcctgtcgcttccctgaagaggaggagggcggctgtgagctgagagtgaagttttccaggtctgccgatgccccagcctataagcagggccagaaccagctgtacaacgagctgaatctgggccggagagaggagtacgacgtgctggataagaggaggggaagggacccagagatgggaggcaagccacggagaaagaacccccaggagggcctgtataatgagctgcagaaggacaagatggccgaggcctacagcgagatcggcatgaagggagagaggcgccggggcaagggacacgatggcctgtatcagggcctgtccacagccaccaaggacacctacgatgcactgcacatgcaggccctgcctccacgcaccagcggatccggcgccacaaatttcagcctgctgaagcaggcaggcgatgtggaggagaacccaggaccacctagaatggcaaggctgggcaattgctctctgacctgggccgccctgatcatcctgctgctgcctggcagcctggaggagtgtggccacatctctgtgagcgccccaatcgtgcacctgggcgaccccatcacagcctcctgcatcatcaagcagaactgttctcacctggatccagagccccagatcctgtggaggctgggagcagagctgcagccaggaggccgccagcagcggctgtctgacggcacccaggagagcatcatcacactgccccacctgaatcacacccaggcctttctgagctgctgtctgaactggggcaattccctgcagatcctggatcaggtggagctgagagccggctatccacccgccatcccacacaacctgtcttgcctgatgaatctgacaacctcctctctgatctgtcagtgggagcctggaccagagacccacctgcccacaagcttcaccctgaagtcttttaagagccggggcaactgccagacacagggcgacagcatcctggattgcgtgcctaaggacggccagtcccactgctgtatcccaagaaagcacctgctgctgtaccagaacatgggcatctgggtgcaggccgagaatgccctgggcacctccatgtctcctcagctgtgcctggacccaatggatgtggtgaagctggagcctccaatgctgcggacaatggatccaagccctgaggcagcaccacctcaggcaggatgcctgcagctgtgctgggagccttggcagccaggcctgcacatcaatcagaagtgtgagctgaggcacaagcctcagaggggagaggcatcctgggcactggtgggcccactgcccctggaggccctgcagtatgagctgtgcggactgctgccagcaacagcatacaccctgcagatcagatgtatcaggtggcctctgccaggacactggagcgactggagcccatccctggagctgagaacaaccgagagggcacctaccgtgcggctggacacatggtggaggcagcggcagctggatccaagaaccgtgcagctgttctggaagcccgtgcctctggaggaggattccggcaggatccagggctatgtggtgtcctggaggccatctggacaggcaggagccatcctgcctctgtgcaacacaaccgagctgagctgtaccttccacctgccatccgaggcacaggaggtggccctggtggcctacaatagcgccggcacctccaggccaacaccagtggtgttttctgagagccgcggacctgccctgaccaggctgcacgcaatggcaagagacccacacagcctgtgggtgggatgggagccaccaaacccttggccacagggatacgtgatcgagtggggactgggacctccatccgcctctaacagcaataagacctggaggatggagcagaacggccgcgccacaggcttcctgctgaaggagaatatccgcccctttcagctgtatgagatcatcgtgacccctctgtaccaggatacaatgggcccaagccagcacgtgtatgcctactcccaggagatggccccttctcacgccccagagctgcacctgaagcacatcggcaagacctgggcacagctggagtgggtgccagagccacctgagctgggcaagtccccactgacacactataccatcttctggacaaacgcccagaatcagtccttttctgccatcctgaacgcaagctcccggggattcgtgctgcacggcctggagcccgcctccctgtaccacatccacctgatggcagcatctcaggcaggagccacaaatagcaccgtgctgacactgatgacactgacccctgagggctctgagctgcacatcatcctgggcctgtttggcctgctgctgctgctgacctgcctgtgcggcacagcctggctgtgctgttctcccaacagaaagaatccactgtggcctagcgtgccagaccctgcacactctagcctgggaagctgggtgccaaccatcatggaggaggatgcctttcagctgccaggactgggaacaccaccaatcaccaagctgacagtgctggaggaggacgagaagaagccagtgccctgggagtcccacaactcctcttga

SEQ ID NO: 7 - DNA, 11743 bp, synthetic construct
  source 1..11743: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagctctagaatggccctgccagtgaccgccctgctgctgccactggccctgctgctgcacgccgccaggcccgacatccagatgacccagaccacaagctccctgagcgcctccctgggcgacagagtgacaatctcctgcagggcctctcaggatatcagcaagtatctgaactggtaccagcagaagcccgatggcaccgtgaagctgctgatctatcacacatcccggctgcactctggcgtgcctagcagattctctggaagcggatccggaaccgactactccctgacaatctctaacctggagcaggaggatatcgccacctatttctgccagcagggcaataccctgccttacacatttggcggcggcaccaagctggagatcacaggcggcggcggctctggcggaggaggaagcggaggaggaggatccgaggtgaagctgcaggagagcggaccaggactggtggcaccttctcagagcctgtccgtgacatgtaccgtgtctggcgtgagcctgccagactacggcgtgtcttggatcaggcagccacctaggaagggcctggagtggctgggcgtgatctggggcagcgagaccacatactataatagcgccctgaagtcccgcctgaccatcatcaaggacaactctaagagccaggtgttcctgaagatgaatagcctgcagacagacgataccgccatctactattgcgccaagcactactattacggcggcagctatgccatggattactggggccagggcacatccgtgaccgtgtctagcaccacaacccctgcaccaaggccaccaaccccagcacctacaatcgcatctcagccactgagcctgcgccccgaggcctgtcggcctgcagcaggaggagcagtgcacaccaggggcctggactttgcctgcgatatctacatctgggcaccactggcaggaacctgtggcgtgctgctgctgagcctggtcatcaccctgtattgcaagagaggcaggaagaagctgctgtacatcttcaagcagcccttcatgaggcccgtgcagacaacccaggaggaggacggctgctcctgtcgcttccctgaggaggaggagggcggctgtgagctgagagtgaagttttccaggtctgccgatgccccagcctataagcagggccagaaccagctgtacaacgagctgaatctgggccggagagaggagtacgacgtgctggataagaggaggggaagggacccagagatgggaggcaagccacggagaaagaacccccaggagggcctgtataatgagctgcagaaggacaagatggccgaggcctactccgagatcggcatgaagggagagaggcgccggggcaagggacacgatggcctgtatcagggcctgtctacagccaccaaggacacctacgatgccctgcacatgcaggccctgcctccaagaaccagcggatccggagcaacaaatttcagcctgctgaagcaggcaggcgacgtggaggagaacccaggaccacctagaatggcaaggctgggcaattgcagcctgacctgggccgccctgatcatcctgctgctgcctggctccctggaggagtgtggccacatctctgtgagcgcccctatcgtgcacctgggcgacccaatcacagcctcctgcatcatcaagcagaactgttctcacctggatccagagccacagatcctgtggaggctgggagcagagctgcagccaggaggccgccagcagcggctgtctgacggcacccaggagagcatcatcacactgccccacctgaatcacacccaggcctttctgagctgctgtctgaactggggcaattccctgcagatcctggatcaggtggagctgagggcaggatatccaccagccatcccacacaacctgagctgcctgatgaatctgacaacctcctctctgatctgtcagtgggagcctggaccagagacccacctgcccacatccttcaccctgaagtcctttaagtctcggggcaactgccagacacagggcgactctatcctggattgcgtgccaaaggacggccagagccactgctgtatccccagaaagcacctgctgctgtaccagaacatgggcatctgggtgcaggccgagaatgccctgggcacctccatgtctccccagctgtgcctggaccctatggatgtggtgaagctggagcctccaatgctgcggacaatggatccaagccctgaggcagcaccacctcaggcaggatgcctgcagctgtgctgggagccttggcagccaggcctgcacatcaatcagaagtgtgagctgaggcacaagcctcagaggggagaggcatcctgggcactggtgggcccactgcccctggaggccctgcagtatgagctgtgcggactgctgccagcaacagcatacaccctgcagatcagatgtatcaggtggcctctgccaggccactggagcgactggagcccttccctggagctgagaacaaccgagagggcaccaaccgtgcggctggacacatggtggaggcagcggcagctggatccaagaaccgtgcagctgttctggaagcccgtgcctctggaggaggattccggcaggatccagggctatgtggtgtcctggaggccttctggacaggcaggagcaatcctgccactgtgcaacacaaccgagctgagctgtaccttccacctgccatccgaggcacaggaggtggccctggtggcatacaatagcgccggcacctcccggccaacaccagtggtgttttctgagagccgcggaccagccctgaccaggctgcacgcaatggcaagagacccacacagcctgtgggtgggatgggagccaccaaacccttggccacagggatacgtgatcgagtggggactgggacctccatccgcctctaacagcaataagacctggaggatggagcagaacggccgcgccacaggcttcctgctgaaggagaatatcagaccctttcagctgtatgagatcatcgtgaccccactgtaccaggatacaatgggccccagccagcacgtgtatgcctactcccaggagatggccccttctcacgccccagagctgcacctgaagcacatcggcaagacctgggcacagctggagtgggtgccagagccacctgagctgggcaagtcccctctgacacactataccatcttctggacaaacgcccagaatcagtccttttctgccatcctgaacgcaagctccaggggattcgtgctgcacggcctggagccagcatctctgtaccacatccacctgatggcagcatctcaggcaggagccacaaatagcaccgtgctgacactgatgacactgacccctgagggctccgagctgcacatcatcctgggcctgtttggcctgctgctgctgctgacctgcctgtgcggcacagcctggctgtgctgttctcctaaccgcaagaatccactgtggcctagcgtgccagaccctgcacactctagcctgggaagctgggtgccaaccatcatggaggaggatgcctttcagctgcctggcctgggcacaccacccatcaccaagctgacagtgctggaggaggacgagaagaagccagtgccctgggagtcccacaactcctctgagacctgcggactgcccaccctggtgcagacatatgtgctgcagggcgacccaagggccgtgtccacccagcctcagagccagtccggcacatctgatcaggtgctgtatggccagctgctgggaagccctacctcccctggaccaggacactacctgagatgcgatagcacccagccactgctggcaggactgacaccatctcctaagagctacgagaacctgtggttccaggcatccccactgggcaccctggtgacaccagccccctctcaggaggacgattgcgtgttcggccctctgctgaattttccactgctgcagggcatcagagtgcacggcatggaggccctgggctccttttgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaac

SEQ ID NO: 8 - DNA, 4047 bp, synthetic construct
  source 1..4047: mol_type = other DNA
atggccctgccagtgaccgccctgctgctgccactggccctgctgctgcacgccgccaggcccgacatccagatgacccagaccacaagctccctgagcgcctccctgggcgacagagtgacaatctcctgcagggcctctcaggatatcagcaagtatctgaactggtaccagcagaagcccgatggcaccgtgaagctgctgatctatcacacatcccggctgcactctggcgtgcctagcagattctctggaagcggatccggaaccgactactccctgacaatctctaacctggagcaggaggatatcgccacctatttctgccagcagggcaataccctgccttacacatttggcggcggcaccaagctggagatcacaggcggcggcggctctggcggaggaggaagcggaggaggaggatccgaggtgaagctgcaggagagcggaccaggactggtggcaccttctcagagcctgtccgtgacatgtaccgtgtctggcgtgagcctgccagactacggcgtgtcttggatcaggcagccacctaggaagggcctggagtggctgggcgtgatctggggcagcgagaccacatactataatagcgccctgaagtcccgcctgaccatcatcaaggacaactctaagagccaggtgttcctgaagatgaatagcctgcagacagacgataccgccatctactattgcgccaagcactactattacggcggcagctatgccatggattactggggccagggcacatccgtgaccgtgtctagcaccacaacccctgcaccaaggccaccaaccccagcacctacaatcgcatctcagccactgagcctgcgccccgaggcctgtcggcctgcagcaggaggagcagtgcacaccaggggcctggactttgcctgcgatatctacatctgggcaccactggcaggaacctgtggcgtgctgctgctgagcctggtcatcaccctgtattgcaagagaggcaggaagaagctgctgtacatcttcaagcagcccttcatgaggcccgtgcagacaacccaggaggaggacggctgctcctgtcgcttccctgaggaggaggagggcggctgtgagctgagagtgaagttttccaggtctgccgatgccccagcctataagcagggccagaaccagctgtacaacgagctgaatctgggccggagagaggagtacgacgtgctggataagaggaggggaagggacccagagatgggaggcaagccacggagaaagaacccccaggagggcctgtataatgagctgcagaaggacaagatggccgaggcctactccgagatcggcatgaagggagagaggcgccggggcaagggacacgatggcctgtatcagggcctgtctacagccaccaaggacacctacgatgccctgcacatgcaggccctgcctccaagaaccagcggatccggagcaacaaatttcagcctgctgaagcaggcaggcgacgtggaggagaacccaggaccacctagaatggcaaggctgggcaattgcagcctgacctgggccgccctgatcatcctgctgctgcctggctccctggaggagtgtggccacatctctgtgagcgcccctatcgtgcacctgggcgacccaatcacagcctcctgcatcatcaagcagaactgttctcacctggatccagagccacagatcctgtggaggctgggagcagagctgcagccaggaggccgccagcagcggctgtctgacggcacccaggagagcatcatcacactgccccacctgaatcacacccaggcctttctgagctgctgtctgaactggggcaattccctgcagatcctggatcaggtggagctgagggcaggatatccaccagccatcccacacaacctgagctgcctgatgaatctgacaacctcctctctgatctgtcagtgggagcctggaccagagacccacctgcccacatccttcaccctgaagtcctttaagtctcggggcaactgccagacacagggcgactctatcctggattgcgtgccaaaggacggccagagccactgctgtatccccagaaagcacctgctgctgtaccagaacatgggcatctgggtgcaggccgagaatgccctgggcacctccatgtctccccagctgtgcctggaccctatggatgtggtgaagctggagcctccaatgctgcggacaatggatccaagccctgaggcagcaccacctcaggcaggatgcctgcagctgtgctgggagccttggcagccaggcctgcacatcaatcagaagtgtgagctgaggcacaagcctcagaggggagaggcatcctgggcactggtgggcccactgcccctggaggccctgcagtatgagctgtgcggactgctgccagcaacagcatacaccctgcagatcagatgtatcaggtggcctctgccaggccactggagcgactggagcccttccctggagctgagaacaaccgagagggcaccaaccgtgcggctggacacatggtggaggcagcggcagctggatccaagaaccgtgcagctgttctggaagcccgtgcctctggaggaggattccggcaggatccagggctatgtggtgtcctggaggccttctggacaggcaggagcaatcctgccactgtgcaacacaaccgagctgagctgtaccttccacctgccatccgaggcacaggaggtggccctggtggcatacaatagcgccggcacctcccggccaacaccagtggtgttttctgagagccgcggaccagccctgaccaggctgcacgcaatggcaagagacccacacagcctgtgggtgggatgggagccaccaaacccttggccacagggatacgtgatcgagtggggactgggacctccatccgcctctaacagcaataagacctggaggatggagcagaacggccgcgccacaggcttcctgctgaaggagaatatcagaccctttcagctgtatgagatcatcgtgaccccactgtaccaggatacaatgggccccagccagcacgtgtatgcctactcccaggagatggccccttctcacgccccagagctgcacctgaagcacatcggcaagacctgggcacagctggagtgggtgccagagccacctgagctgggcaagtcccctctgacacactataccatcttctggacaaacgcccagaatcagtccttttctgccatcctgaacgcaagctccaggggattcgtgctgcacggcctggagccagcatctctgtaccacatccacctgatggcagcatctcaggcaggagccacaaatagcaccgtgctgacactgatgacactgacccctgagggctccgagctgcacatcatcctgggcctgtttggcctgctgctgctgctgacctgcctgtgcggcacagcctggctgtgctgttctcctaaccgcaagaatccactgtggcctagcgtgccagaccctgcacactctagcctgggaagctgggtgccaaccatcatggaggaggatgcctttcagctgcctggcctgggcacaccacccatcaccaagctgacagtgctggaggaggacgagaagaagccagtgccctgggagtcccacaactcctctgagacctgcggactgcccaccctggtgcagacatatgtgctgcagggcgacccaagggccgtgtccacccagcctcagagccagtccggcacatctgatcaggtgctgtatggccagctgctgggaagccctacctcccctggaccaggacactacctgagatgcgatagcacccagccactgctggcaggactgacaccatctcctaagagctacgagaacctgtggttccaggcatccccactgggcaccctggtgacaccagccccctctcaggaggacgattgcgtgttcggccctctgctgaattttccactgctgcagggcatcagagtgcacggcatggaggccctgggctccttttga

SEQ ID NO: 9 - DNA, 11584 bp, synthetic construct
  source 1..11584: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagctctagaatggcactgcctgtgaccgccctgctgctgccactggccctgctgctgcacgcagcaaggccagacatccagatgacccagaccacaagctccctgagcgcctccctgggcgacagggtgacaatcagctgcagggcctcccaggatatctctaagtatctgaactggtaccagcagaagccagatggcaccgtgaagctgctgatctatcacacaagcaggctgcactccggagtgccatctcgcttctctggaagcggatccggaaccgactacagcctgacaatctccaacctggagcaggaggatatcgccacctatttctgccagcagggcaataccctgccttacacatttggcggcggcaccaagctggagatcacaggcggaggaggatccggaggaggaggctctggcggcggcggcagcgaggtgaagctgcaggagagcggacctggactggtggcaccatctcagagcctgtccgtgacatgtaccgtgtctggcgtgagcctgcccgactacggcgtgtcttggatcagacagccacctaggaagggcctggagtggctgggcgtgatctggggcagcgagaccacatactataactctgccctgaagagccgcctgaccatcatcaaggacaactctaagagccaggtgttcctgaagatgaattccctgcagacagacgataccgccatctactattgcgccaagcactactattacggcggctcttatgccatggattactggggccagggcacaagcgtgaccgtgtctagcaccacaaccccagcaccaaggccaccaacccctgcaccaacaatcgcatcccagccactgtctctgagacctgaggcatgtaggccagcagcaggaggagcagtgcacaccagaggcctggactttgcctgcgatatctacatctgggcaccactggcaggaacctgtggcgtgctgctgctgagcctggtcatcaccctgtattgcaagcgcggccggaagaagctgctgtacatcttcaagcagccctttatgcggcctgtgcagacaacccaggaggaggacggctgctcctgtagattcccagaggaggaggagggaggatgtgagctgcgcgtgaagttttcccggtctgccgatgcccctgcctataagcagggccagaaccagctgtacaacgagctgaatctgggccggagagaggagtacgacgtgctggataagaggaggggaagggacccagagatgggaggcaagccccggagaaagaaccctcaggagggcctgtataatgagctgcagaaggacaagatggccgaggcctactccgagatcggcatgaagggagagaggcgccggggcaagggacacgatggcctgtatcagggcctgtctacagccaccaaggacacctacgatgccctgcacatgcaggccctgcctccaagaaccagcggatccggagcaacaaatttcagcctgctgaagcaggcaggcgacgtggaggagaacccaggaccacctaggatggcaaggctgggcaattgcagcctgacctgggccgccctgatcatcctgctgctgccaggatccctggaggagtgtggccacatctctgtgagcgccccaatcgtgcacctgggcgaccctatcacagccagctgcatcatcaagcagaactgttcccacctggatcccgagcctcagatcctgtggaggctgggagcagagctgcagcctggaggcagacagcagaggctgtccgacggaacccaggagtctatcatcacactgccacacctgaatcacacccaggcctttctgtcttgctgtctgaactggggcaatagcctgcagatcctggatcaggtggagctgagggcaggatatccaccagccatccctcacaacctgagctgcctgatgaatctgacaacctcctctctgatctgtcagtgggagccaggaccagagacccacctgccaacatccttcaccctgaagtcctttaagtctaggggcaactgccagacacagggcgactccatcctggattgcgtgcctaaggacggccagtctcactgctgtatcccacgcaagcacctgctgctgtaccagaacatgggcatctgggtgcaggccgagaatgccctgggcacctccatgtctccacagctgtgcctggaccccatggatgtggtgaagctggagcctccaatgctgcggacaatggatcctagcccagaggcagcaccacctcaggcaggatgcctgcagctgtgctgggagccatggcagccaggcctgcacatcaatcagaagtgtgagctgagacacaagccccagaggggagaggcatcctgggcactggtgggacccctgcctctggaggccctgcagtatgagctgtgcggactgctgcctgcaacagcatacaccctgcagatcaggtgtatcaggtggccactgccaggacactggagcgactggagcccatccctggagctgaggacaaccgagagggcaccaaccgtgaggctggacacatggtggagacagaggcagctggatcctcgcaccgtgcagctgttctggaagcctgtgccactggaggaggattccggccggatccagggctatgtggtgagctggagaccatccggacaggcaggagccatcctgcctctgtgcaacacaaccgagctgtcttgtaccttccacctgccaagcgaggcacaggaggtggccctggtggcatacaattctgccggcaccagccggcccacacctgtggtgttttctgagagcagaggacccgccctgaccaggctgcacgcaatggcaagggaccctcacagcctgtgggtgggatgggagccaccaaacccatggccacagggatacgtgatcgagtggggactgggacctccatccgcctctaacagcaataagacctggcggatggagcagaacggcagagccacaggcttcctgctgaaggagaatatcagaccctttcagctgtatgagatcatcgtgacccctctgtaccaggatacaatgggcccaagccagcacgtgtatgcctacagccaggagatggcaccatcccacgcaccagagctgcacctgaagcacatcggcaagacctgggcacagctggagtgggtgccagagccacctgagctgggcaagtccccactgacacactataccatcttctggacaaacgcccagaatcagtccttttctgccatcctgaacgcaagctccaggggattcgtgctgcacggcctggagccagcatccctgtaccacatccacctgatggcagcatcccaggcaggagcaacaaattctaccgtgctgacactgatgacactgacccccgagggctctgagctgcacatcatcctgggcctgtttggcctgctgctgctgctgacctgcctgtgcggcacagcctggctgtgctgtagccctaaccgcaagaatcctctgtggccatccgtgcctgacccagcccactctagcctgggcagctgggtgccaaccatcatggaggaggatgcctttcagctgccaggactgggaacaccaccaatcaccaagctgacagtgctggaggaggacgagaagaagcccgtgccttgggagtctcacaactcctctgagacctgcggactgcccaccctggtgcagacatatgtgctgcagggcgatccaagagccgtgagcacccagccacagagccagtccggcacatccgaccaggcaggacctccaagaaggtctgcctacttcaaggatcagatcatgctgcaccctgccccccctaatggcctgctgtgcctgtttccaatcacctccgtgctgtgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaac

SEQ ID NO: 10 - DNA, 3888 bp, synthetic construct
  source 1..3888: mol_type = other DNA
atggcactgcctgtgaccgccctgctgctgccactggccctgctgctgcacgcagcaaggccagacatccagatgacccagaccacaagctccctgagcgcctccctgggcgacagggtgacaatcagctgcagggcctcccaggatatctctaagtatctgaactggtaccagcagaagccagatggcaccgtgaagctgctgatctatcacacaagcaggctgcactccggagtgccatctcgcttctctggaagcggatccggaaccgactacagcctgacaatctccaacctggagcaggaggatatcgccacctatttctgccagcagggcaataccctgccttacacatttggcggcggcaccaagctggagatcacaggcggaggaggatccggaggaggaggctctggcggcggcggcagcgaggtgaagctgcaggagagcggacctggactggtggcaccatctcagagcctgtccgtgacatgtaccgtgtctggcgtgagcctgcccgactacggcgtgtcttggatcagacagccacctaggaagggcctggagtggctgggcgtgatctggggcagcgagaccacatactataactctgccctgaagagccgcctgaccatcatcaaggacaactctaagagccaggtgttcctgaagatgaattccctgcagacagacgataccgccatctactattgcgccaagcactactattacggcggctcttatgccatggattactggggccagggcacaagcgtgaccgtgtctagcaccacaaccccagcaccaaggccaccaacccctgcaccaacaatcgcatcccagccactgtctctgagacctgaggcatgtaggccagcagcaggaggagcagtgcacaccagaggcctggactttgcctgcgatatctacatctgggcaccactggcaggaacctgtggcgtgctgctgctgagcctggtcatcaccctgtattgcaagcgcggccggaagaagctgctgtacatcttcaagcagccctttatgcggcctgtgcagacaacccaggaggaggacggctgctcctgtagattcccagaggaggaggagggaggatgtgagctgcgcgtgaagttttcccggtctgccgatgcccctgcctataagcagggccagaaccagctgtacaacgagctgaatctgggccggagagaggagtacgacgtgctggataagaggaggggaagggacccagagatgggaggcaagccccggagaaagaaccctcaggagggcctgtataatgagctgcagaaggacaagatggccgaggcctactccgagatcggcatgaagggagagaggcgccggggcaagggacacgatggcctgtatcagggcctgtctacagccaccaaggacacctacgatgccctgcacatgcaggccctgcctccaagaaccagcggatccggagcaacaaatttcagcctgctgaagcaggcaggcgacgtggaggagaacccaggaccacctaggatggcaaggctgggcaattgcagcctgacctgggccgccctgatcatcctgctgctgccaggatccctggaggagtgtggccacatctctgtgagcgccccaatcgtgcacctgggcgaccctatcacagccagctgcatcatcaagcagaactgttcccacctggatcccgagcctcagatcctgtggaggctgggagcagagctgcagcctggaggcagacagcagaggctgtccgacggaacccaggagtctatcatcacactgccacacctgaatcacacccaggcctttctgtcttgctgtctgaactggggcaatagcctgcagatcctggatcaggtggagctgagggcaggatatccaccagccatccctcacaacctgagctgcctgatgaatctgacaacctcctctctgatctgtcagtgggagccaggaccagagacccacctgccaacatccttcaccctgaagtcctttaagtctaggggcaactgccagacacagggcgactccatcctggattgcgtgcctaaggacggccagtctcactgctgtatcccacgcaagcacctgctgctgtaccagaacatgggcatctgggtgcaggccgagaatgccctgggcacctccatgtctccacagctgtgcctggaccccatggatgtggtgaagctggagcctccaatgctgcggacaatggatcctagcccagaggcagcaccacctcaggcaggatgcctgcagctgtgctgggagccatggcagccaggcctgcacatcaatcagaagtgtgagctgagacacaagccccagaggggagaggcatcctgggcactggtgggacccctgcctctggaggccctgcagtatgagctgtgcggactgctgcctgcaacagcatacaccctgcagatcaggtgtatcaggtggccactgccaggacactggagcgactggagcccatccctggagctgaggacaaccgagagggcaccaaccgtgaggctggacacatggtggagacagaggcagctggatcctcgcaccgtgcagctgttctggaagcctgtgccactggaggaggattccggccggatccagggctatgtggtgagctggagaccatccggacaggcaggagccatcctgcctctgtgcaacacaaccgagctgtcttgtaccttccacctgccaagcgaggcacaggaggtggccctggtggcatacaattctgccggcaccagccggcccacacctgtggtgttttctgagagcagaggacccgccctgaccaggctgcacgcaatggcaagggaccctcacagcctgtgggtgggatgggagccaccaaacccatggccacagggatacgtgatcgagtggggactgggacctccatccgcctctaacagcaataagacctggcggatggagcagaacggcagagccacaggcttcctgctgaaggagaatatcagaccctttcagctgtatgagatcatcgtgacccctctgtaccaggatacaatgggcccaagccagcacgtgtatgcctacagccaggagatggcaccatcccacgcaccagagctgcacctgaagcacatcggcaagacctgggcacagctggagtgggtgccagagccacctgagctgggcaagtccccactgacacactataccatcttctggacaaacgcccagaatcagtccttttctgccatcctgaacgcaagctccaggggattcgtgctgcacggcctggagccagcatccctgtaccacatccacctgatggcagcatcccaggcaggagcaacaaattctaccgtgctgacactgatgacactgacccccgagggctctgagctgcacatcatcctgggcctgtttggcctgctgctgctgctgacctgcctgtgcggcacagcctggctgtgctgtagccctaaccgcaagaatcctctgtggccatccgtgcctgacccagcccactctagcctgggcagctgggtgccaaccatcatggaggaggatgcctttcagctgccaggactgggaacaccaccaatcaccaagctgacagtgctggaggaggacgagaagaagcccgtgccttgggagtctcacaactcctctgagacctgcggactgcccaccctggtgcagacatatgtgctgcagggcgatccaagagccgtgagcacccagccacagagccagtccggcacatccgaccaggcaggacctccaagaaggtctgcctacttcaaggatcagatcatgctgcaccctgccccccctaatggcctgctgtgcctgtttccaatcacctccgtgctgtga

SEQ ID NO: 11 - DNA, 2148 bp, synthetic construct
  source 1..2148: mol_type = other DNA
atggcaaggctgggaaactgcagcctgacttgggctgccctgatcatcctgctgctccccggaagtctggaggagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacggatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgtcccactccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcctcctcccgtggctttgtcctccatggcctggagcccgccagtctgtatcacatccacctcatggctgccagccaggctggggccaccaacagtacagtcctcaccctgatgaccttgaccccagaggggtcggagctacacatcatcctgggcctgttcggcctcctgctgttgctcacctgcctctgtggaactgcctggctctgttgcagccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcatag

SEQ ID NO: 12 - DNA, 2511 bp, synthetic construct
  source 1..2511: mol_type = other DNA
atggcaaggctgggaaactgcagcctgacttgggctgccctgatcatcctgctgctccccggaagtctggaggagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacggatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgtcccactccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcctcctcccgtggctttgtcctccatggcctggagcccgccagtctgtatcacatccacctcatggctgccagccaggctggggccaccaacagtacagtcctcaccctgatgaccttgaccccagaggggtcggagctacacatcatcctgggcctgttcggcctcctgctgttgctcacctgcctctgtggaactgcctggctctgttgcagccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcagagacctgtggcctccccactctggtccagacctatgtgctccagggggacccaagagcagtttccacccagccccaatcccagtctggcaccagcgatcaggtcctttatgggcagctgctgggcagccccacaagcccagggccagggcactatctccgctgtgactccactcagcccctcttggcgggcctcacccccagccccaagtcctatgagaacctctggttccaggccagccccttggggaccctggtaaccccagccccaagccaggaggacgactgtgtctttgggccactgctcaacttccccctcctgcaggggatccgggtccatgggatggaggcgctggggagcttctag

SEQ ID NO: 13 - DNA, 2100 bp, synthetic construct
  source 1..2100: mol_type = other DNA
gacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcactcgacgaatctaagtacggaccgccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgcggagcaagcggagcagaggcggccacagcgactacatgaacatgacccccagacggcctggccccacccggaagcactaccagccctacgccccacccagggactttgccgcctacagaagcaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 14 - DNA, 2352 bp, synthetic construct
  source 1..2352: mol_type = other DNA
atggcaaggctgggaaactgcagcctgacttgggctgccctgatcatcctgctgctccccggaagtctggaggagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacggatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgtcccactccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcctcctcccgtggctttgtcctccatggcctggagcccgccagtctgtatcacatccacctcatggctgccagccaggctggggccaccaacagtacagtcctcaccctgatgaccttgaccccagaggggtcggagctacacatcatcctgggcctgttcggcctcctgctgttgctcacctgcctctgtggaactgcctggctctgttgcagccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcagagacctgtggcctccccactctggtccagacctatgtgctccagggggacccaagagcagtttccacccagccccaatcccagtctggcaccagcgatcaggctgggcctcccaggcgatctgcatactttaaggaccagatcatgctccatccagccccacccaatggccttttgtgcttgtttcctataacttcagtattgtaa

SEQ ID NO: 15 - protein, 5 aa, synthetic construct
  REPEAT 1..5: note = repeat n times, where n represents an integer of at least 1
GSGGS

SEQ ID NO: 16 - protein, 4 aa, synthetic construct
  REPEAT 1..4: note = repeat n times, where n represents an integer of at least 1
GGGS

SEQ ID NO: 17 - protein, 5 aa, synthetic construct
  REPEAT 1..5: note = repeat n times, where n represents an integer of at least 1
GGGGS

SEQ ID NO: 18 - protein, 4 aa, synthetic construct
GGSG

SEQ ID NO: 19 - protein, 5 aa, synthetic construct
GGSGG

SEQ ID NO: 20 - protein, 5 aa, synthetic construct
GSGSG

SEQ ID NO: 21 - protein, 5 aa, synthetic construct
GSGGG

SEQ ID NO: 22 - protein, 5 aa, synthetic construct
GGGSG

SEQ ID NO: 23 - protein, 5 aa, synthetic construct
GSSSG

SEQ ID NO: 24 - protein, 5 aa, synthetic construct
GGGGS

SEQ ID NO: 25 - protein, 15 aa, synthetic construct
GGGGSGGGGSGGGGS

SEQ ID NO: 26 - DNA, 45 bp, synthetic construct
  source 1..45: mol_type = other DNA
ggtggcggtggctcgggcggtggtgggtcgggtggcggcggatct

SEQ ID NO: 27 - protein, 5 aa, synthetic construct
DKTHT

SEQ ID NO: 28 - protein, 4 aa, synthetic construct
CPPC

SEQ ID NO: 29 - protein, 15 aa, synthetic construct
CPEPKSCDTPPPCPR

SEQ ID NO: 30 - protein, 12 aa, synthetic construct
ELKTPLGDTTHT

SEQ ID NO: 31 - protein, 10 aa, synthetic construct
KSCDKTHTCP

SEQ ID NO: 32 - protein, 7 aa, synthetic construct
KCCVDCP

SEQ ID NO: 33 - protein, 7 aa, synthetic construct
KYGPPCP

SEQ ID NO: 34 - protein, 15 aa, synthetic construct
EPKSCDKTHTCPPCP

SEQ ID NO: 35 - protein, 12 aa, synthetic construct
ERKCCVECPPCP

SEQ ID NO: 36 - protein, 17 aa, synthetic construct
ELKTPLGDTTHTCPRCP

SEQ ID NO: 37 - protein, 12 aa, synthetic construct
SPNMVPHAHHAQ

SEQ ID NO: 38 - protein, 15 aa, synthetic construct
EPKSCDKTYTCPPCP